(19) United States
(12) Patent Application Publication
(10) Pub. No.: US 2026/0259498 A1
(43) Pub. Date: Sep. 3, 2026

(54) RADIATION-SENSITIVE COMPOSITION, METHOD FOR FORMING PATTERN, POLYMER, AND COMPOUND

(71) Applicant: JSR CORPORATION, Tokyo (JP)

(72) Inventors: Daichi WATANABE, Tokyo (JP); Masafumi ITAGAKI, Tokyo (JP); Yoshiki NONOYAMA, Tokyo (JP); Tomoya CHOSA, Tokyo (JP)

(73) Assignee: JSR CORPORATION, Tokyo (JP)

(21) Appl. No.: 19/648,233

(22) Filed: Apr. 15, 2026

Related U.S. Application Data
(63) Continuation-in-part of application No. PCT/JP2024/034915, filed on Sep. 30, 2024.

(30) Foreign Application Priority Data
Oct. 19, 2023 (JP) 2023-180563

(51) Int. Cl. G03F 7/039; C07D 307/68; C07D 333/40; C07D 333/54; C07D 407/12; C07D 409/12; C08F 212/14; C08F 220/18; C08F 220/22; C08F 220/28; C08F 220/30; C08F 220/40; G03F 7/00; G03F 7/004; G03F 7/20

(52) U.S. Cl. CPC G03F 7/0397; C07D 307/68; C07D 333/40; C07D 333/54; C07D 407/12; C07D 409/12; C08F 212/22; C08F 220/1806; C08F 220/1807; C08F 220/1809; C08F 220/1811; C08F 220/1818; C08F 220/22; C08F 220/281; C08F 220/301; C08F 220/40; G03F 7/0045; C08F 2800/10; G03F 7/2002; G03F 7/70008

(57) ABSTRACT

A radiation-sensitive composition includes: a polymer (A) including a structural unit (I) represented by formula (1); and a solvent (B). The polymer (A) includes a radiation-sensitive acid-generating structural unit (IV), or the composition includes a radiation-sensitive acid generator (C) other than the polymer (A), or both. At least one selected from the group consisting of the polymer (A) and the radiation-sensitive acid generator (C) includes an iodo group. R¹ is a hydrogen atom, a fluorine atom, a methyl group, or a trifluoromethyl group; Ar represents a furan ring, a thiophene ring, or an aromatic heterocycle having 9 to 20 ring members including a furan ring or a thiophene ring; and R² is a hydrogen atom or a monovalent organic group having 1 to 20 carbon atoms.

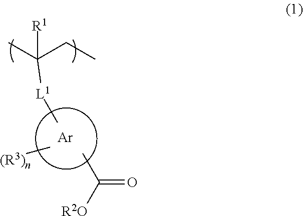

### CROSS-REFERENCE TO RELATED APPLICATIONS

The present application is a continuation-in-part application of International Patent Application No. PCT/JP2024/034915 filed Sep. 30, 2024, which claims priority to Japanese Patent Application No. 2023-180563 filed Oct. 19, 2023. The contents of these applications are incorporated herein by reference in their entirety.

### BACKGROUND OF THE DISCLOSURE

### Technical Field

The present disclosure relates to a radiation-sensitive composition, a method for forming a pattern, a polymer and a compound.

### Background Art

A photolithography technique performed using a resist composition has been utilized for formation of a fine circuit on a semiconductor device. As the representative procedure, for example, a resist pattern is formed on a substrate by generating an acid by irradiating the coating of the resist composition with radioactive ray through a mask pattern, and then generating a difference in solubility of resin into an alkaline or organic developer between an exposed part and a non-exposed part through a reaction in the presence of the acid as a catalyst.

In the photolithography technique, pattern miniaturization is promoted by using short-wavelength radiation such as ArF excimer laser, or by combining such radiation with an immersion exposure method (liquid immersion lithography).

As a next-generation technology, further short-wavelength radiation, such as an electron beam, an X-ray, and an extreme ultraviolet ray (EUV) is being utilized, and a resist material having an enhanced efficiency of absorbing such radiation is also being studied (JP-A-2008-250227).

### SUMMARY

The present invention relates to, in one embodiment, a radiation-sensitive composition containing a polymer (A) including a structural unit (I) represented by a following formula (1), and a solvent (B),
wherein the polymer (A) contains a radiation-sensitive acid-generating structural unit (IV), or the composition comprises a radiation-sensitive acid generator (C) other than the polymer (A), or both, and
at least one selected from the group consisting of the polymer (A) and the radiation-sensitive acid generator (C) contains an iodo group:

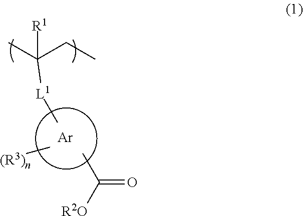

wherein in the formula (1),
R¹ is a hydrogen atom, a fluorine atom, a methyl group, or a trifluoromethyl group;
L¹ is a single bond or a divalent linking group;
Ar represents a furan ring, a thiophene ring, or an aromatic heterocycle having 9 to 20 ring members containing a furan ring or a thiophene ring;
R² is a hydrogen atom or a monovalent organic group having 1 to 20 carbon atoms;
R³ is a halogen atom, a hydroxy group, a cyano group, a nitro group, an amino group, a thiol group, or a monovalent organic group having 1 to 20 carbon atoms; and
n is an integer of 0 to 4; and when n is 2 or more, a plurality of R³s are identical to or different from each other.

The present invention relates to, in another embodiment,
a pattern forming method, including
directly or indirectly applying the above-described radiation-sensitive composition onto a substrate to form a resist film,
exposing the resist film to light, and
developing the exposed resist film with a developer.

The present invention relates to, in another embodiment, a polymer containing a structural unit (I) represented by a following formula (1):

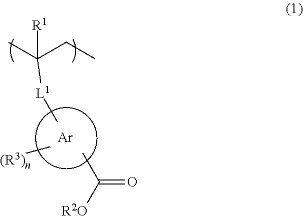

wherein in the formula (1),
R¹ is a hydrogen atom, a fluorine atom, a methyl group, or a trifluoromethyl group;
L¹ is a single bond or a divalent linking group;
Ar represents a furan ring, a thiophene ring, or an aromatic heterocycle having 9 to 20 ring members containing a furan ring or a thiophene ring;
R² is a hydrogen atom or a monovalent organic group having 1 to 20 carbon atoms;
R³ is a halogen atom, a hydroxy group, a cyano group, a nitro group, an amino group, a thiol group, or a monovalent organic group having 1 to 20 carbon atoms;
n is an integer of 0 to 4; and when n is 2 or more, a plurality of R³s are identical to or different from each other.

The present invention relates to, in another embodiment, following compounds (hereinafter, also simply referred to as a “compound 1 to 5”).

### (Compound 1) a Compound Represented by a Following Formula (1-1):

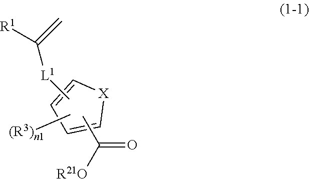

wherein in the formula (1-1),
X represents —S— or —O—;
R¹ is a hydrogen atom, a fluorine atom, a methyl group, or a trifluoromethyl group;
L¹ is a single bond or a divalent linking group;
R²¹ is an acid-dissociable group represented by a following formula (2-1), provided that when X is —S—, a total number of carbon atoms of L¹ and R²¹ is 5 or more:

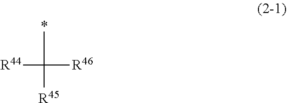

wherein in the formula (2-1),
R⁴⁴ is a hydrogen atom or a monovalent organic group having 1 to 10 carbon atoms;
R⁴⁵ and R⁴⁶ each independently represent a monovalent organic group having 1 to 10 carbon atoms, or R⁴⁵ and R⁴⁶ taken together represent a divalent alicyclic group or heterocyclic group having 3 to 10 ring members together with the carbon atom to which R⁴⁵ and R⁴⁶ are bonded,
provided that when R⁴⁴, R⁴⁵, or R⁴⁶ has a double bond other than an aromatic ring, two carbon atoms composing the double bond are bonded to 0, 2, or 3 hydrogen atoms in total; and
* represents a bond bonded to an oxygen atom,
R³ is a halogen atom, a hydroxy group, a cyano group, a nitro group, an amino group, a thiol group, or a monovalent organic group having 1 to 20 carbon atoms;
n1 is an integer of 0 to 2; and when n1 is 2, two R³s are identical to or different from each other.

### (Compound 2) a Compound Represented by a Following Formula (1-2-1) or a Following Formula (1-2-2):

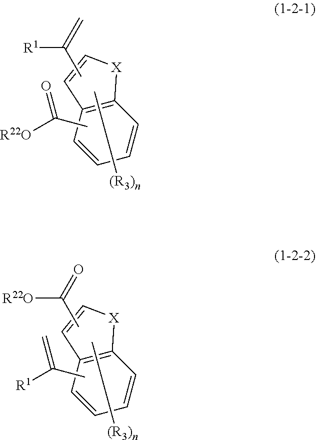

wherein in the formula (1-2-1) and the formula (1-2-2),
X represents —S— or —O—;
R¹ is a hydrogen atom, a fluorine atom, a methyl group, or a trifluoromethyl group;
R²² is an acid-dissociable group represented by a following formula (2-2), provided that a number of carbon atoms of R²² is 6 or more:

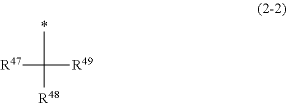

wherein in the formula (2-2),
R⁴⁷ is a hydrogen atom or a monovalent organic group having 1 to 10 carbon atoms;
R⁴⁸ and R⁴⁹ each independently represent a monovalent organic group having 1 to 10 carbon atoms, or R⁴⁸ and R⁴⁹ taken together represent a divalent alicyclic group or heterocyclic group having 3 to 10 ring members together with the carbon atom to which R⁴⁸ and R⁴⁹ are bonded,
provided that when R⁴⁷, R⁴⁸, or R⁴⁹ has a double bond other than a double bond of an aromatic ring, two carbon atoms bonded by the double bond are bonded to 0, 1, or 3 hydrogen atoms in total; and
* represents a bond bonded to an oxygen atom,
R³ is a halogen atom, a hydroxy group, a cyano group, a nitro group, an amino group, a thiol group, or a monovalent organic group having 1 to 20 carbon atoms;
n is an integer of 0 to 4; and when n is 2 or more, a plurality of R³s are identical to or different from each other.

### (Compound 3) a Compound Represented by a Following Formula (1-3):

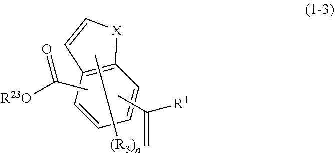

wherein in the formula (1-3),
X represents —S— or —O—;
R¹ is a hydrogen atom, a fluorine atom, a methyl group, or a trifluoromethyl group;
R²³ is a monovalent organic group having 3 or more carbon atoms;
R³ is a halogen atom, a hydroxy group, a cyano group, a nitro group, an amino group, a thiol group, or a monovalent organic group having 1 to 20 carbon atoms;
n is an integer of 0 to 4; and when n is 2 or more, a plurality of R³s are identical to or different from each other.

### (Compound 4) a Compound Represented by a Following Formula (1-4):

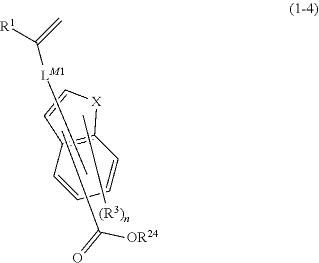

wherein in the formula (1-4),
X represents —S— or —O—;
R¹ is a hydrogen atom, a fluorine atom, a methyl group, or a trifluoromethyl group;
L^{M1} is a divalent linking group;
R²⁴ is a group represented by a following formula (2-3):

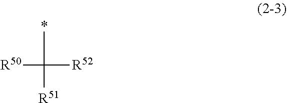

wherein in the formula (2-3),
R⁵⁰ is a hydrogen atom or a monovalent organic group having 1 to 10 carbon atoms;
R⁵¹ and R⁵² each independently represent a monovalent organic group having 1 to 10 carbon atoms, or R⁵¹ and R⁵² taken together represent a divalent alicyclic group or heterocyclic group having 3 to 10 ring members together with the carbon atom to which R⁵¹ and R⁵² are bonded; and
* represents a bond bonded to an oxygen atom,
R³ is a halogen atom, a hydroxy group, a cyano group, a nitro group, an amino group, a thiol group, or a monovalent organic group having 1 to 20 carbon atoms;
n is an integer of 0 to 4; and when n is 2 or more, a plurality of R³s are identical to or different from each other.

### (Compound 5) a Compound Represented by a Following Formula (1-5):

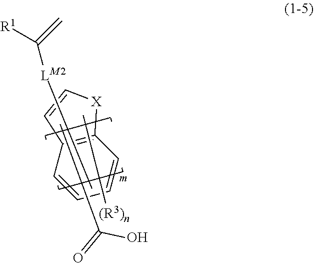

wherein in the formula (1-5),
X represents —S— or —O—;
R¹ is a hydrogen atom, a fluorine atom, a methyl group, or a trifluoromethyl group;
L^{M2} is a divalent linking group, provided that when m=0 and X is —O—, L^{M2} is a group represented by a following formula (3):

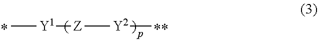

wherein in the formula (3),
Y¹ and Y² are each independently a single bond, —O—, —CO—, —CO—O—, —O—CO—, —O—CO—O—, —CO—NH—, —NH—CO—, or —S—;
Z is a divalent hydrocarbon group having 1 to 10 carbon atoms, provided that when p is 0, Y¹ is —O—, —CO—, —CO—O—, —O—CO—, —O—CO—O—, —CO—NH—, —NH—CO—, or —S—;
p is an integer of 0 to 2; and
* represents a bond bonded to a carbon atom of the vinyl group in the formula (1-5), and ** represents a bond bonded to a carbon atom of the heterocyclic ring in the formula (1-5),
R³ is a halogen atom, a hydroxy group, a cyano group, a nitro group, an amino group, a thiol group, or a monovalent organic group having 1 to 20 carbon atoms;
m is 0 or 1;
n is an integer of 0 to 4; and when n is 2 or more, a plurality of R³s are identical to or different from each other.

### DESCRIPTION OF THE EMBODIMENTS

As used herein, the words “a” and “an” and the like carry the meaning of “one or more.” When an amount, concentration, or other value or parameter is given as a range, and/or its description includes a list of upper and lower values, this is to be understood as specifically disclosing all integers and fractions within the given range, and all ranges formed from any pair of any upper and lower values, regardless of whether subranges are separately disclosed. Where a range of numerical values is recited herein, unless otherwise stated, the range is intended to include the endpoints thereof, as well as all integers and fractions within the range. As an example, a stated range of 1-10 fully describes and includes the independent subrange 3.4-7.2 as does the following list of values: 1, 4, 6, 10.

Even in the above-described next-generation technology, various resist performances equivalent to or higher than conventional performances are required in sensitivity and critical dimension uniformity (CDU), which is an index of uniformity of a line width and a hole diameter, development defects, and the like.

According to the radiation-sensitive composition of the present disclosure, a resist film that satisfies sensitivity and CDU and in which the occurrence of development defects is suppressed, when a next-generation technology is applied, can be constructed. The reason for this is not clear, but is presumed as follows. Since the polymer (A) contains a structural unit having a specific aromatic heterocycle, hydrophilicity is improved, solubility of the polymer (A) in a developer is improved, and as a result, CDU and development defect-suppressing properties can be improved. In addition, since at least one of the polymer (A) or the radiation-sensitive acid generator (C) contains an iodo group, the secondary electron generation efficiency is enhanced, and as a result, the radiation-sensitive composition has high sensitivity. It is presumed that due to these combined actions, sensitivity, CDU, and development defect-suppressing properties can be exhibited.

In the pattern forming method of the present disclosure, since the radiation-sensitive composition capable of forming a resist film having excellent sensitivity and CDU and suppressed occurrence of development defects is used, a high-quality resist pattern can be efficiently formed.

Using a radiation-sensitive composition containing the polymer (A) of the present disclosure, a resist film having excellent sensitivity and CDU and suppressed development defects can be formed.

Using a radiation-sensitive composition containing the polymer including a structural unit derived from at least one of compounds 1 to 5 of the present disclosure, a resist film having excellent sensitivity and CDU and suppressed development defects can be formed.

Hereinafter, embodiments of the present invention will be described in detail, but the present invention is not limited to these embodiments. Combinations of suitable embodiments are also preferable.

### <<Radiation-Sensitive Composition>>

The radiation-sensitive composition (hereinafter, also simply referred to as the “composition”) according to the present embodiment contains a polymer (A) and a solvent (B), and contains in the polymer (A) a radiation-sensitive acid-generating structural unit (IV), or contains a radiation-sensitive acid generator (C) other than the polymer (A), or contains both of them, and contains an iodo group in at least one selected from the group consisting of the polymer (A) and the radiation-sensitive acid generator (C). The composition may contain other optional components as long as the effects of the present disclosure are not impaired. The radiation-sensitive composition contains a polymer (A) containing the structural unit (I) represented by the formula (1), and contains in the polymer (A) a radiation-sensitive acid-generating structural unit, or contains a radiation-sensitive acid generator (C) other than the polymer (A), or contains both of them, and further contains an iodo group in at least one selected from the group consisting of the polymer (A) and the radiation-sensitive acid generator (C), whereby the resist film obtained from the radiation-sensitive composition can exhibit sensitivity and CDU at a higher level, and occurrence of development defects can be suppressed.

### <Polymer (A)>

The polymer (A) is an assembly of polymerized chains containing a structural unit (I) represented by the following formula (1) (hereinafter, this assembly is also referred to as a “base polymer (A)”). It is only required that the entire polymer composing the polymer (A) contains the structural unit (I). The polymer (A) may also contain a structural unit other than the structural unit (I). The polymer (A) may be a polymer having a radiation-sensitive acid-generating structural unit (IV) that generates an acid through exposure to light (radiation-sensitive acid-generating polymer (A1)), or may be a polymer having no radiation-sensitive acid-generating structural unit (IV).

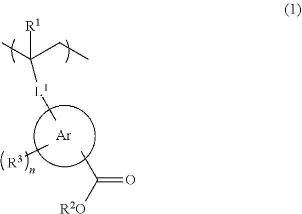

wherein in the formula (1),
R¹ is a hydrogen atom, a fluorine atom, a methyl group, or a trifluoromethyl group;
L¹ is a single bond or a divalent linking group;
Ar represents a furan ring, a thiophene ring, or an aromatic heterocycle having 9 to 20 ring members containing a furan ring or a thiophene ring;
R² is a hydrogen atom or a monovalent organic group having 1 to 20 carbon atoms;
R³ is a halogen atom, a hydroxy group, a cyano group, a nitro group, an amino group, a thiol group, or a monovalent organic group having 1 to 20 carbon atoms;
n is an integer of 0 to 4; and when n is 2 or more, a plurality of R³s are identical to or different from each other.

The radiation-sensitive acid-generating polymer (A1) (hereinafter, this polymer is also referred to as the “base polymer (A1)”) is an aggregate of polymer chains containing a structural unit (I) represented by the following formula (1) and a radiation-sensitive acid-generating structural unit (IV), and is a component that generates an acid through exposure to light. The structural unit (I) and the structural unit (IV) may be contained in the same polymer chain, or alternatively, the structural unit (I) may be contained in one polymer chain and the structural unit (IV) may be contained in another polymer chain. It is merely required that the whole polymer constituting the radiation-sensitive acid-generating polymer (A1) contains the structural unit (I) and the structural unit (IV). The radiation-sensitive acid-generating polymer (A1) may contain a structural unit other than the structural unit (I) and the structural unit (IV).

In this specification, the onium salt structure in the radiation-sensitive acid-generating structural unit (IV) is incorporated as a part of a polymer is referred to as a “radiation-sensitive acid-generating polymer”, and a low molecular weight form (released from a polymer) in which the onium salt structure is present alone as a compound is referred to as a “radiation-sensitive acid generator”.

### (Structural Unit (I))

The polymer (A) contains a structural unit (I) represented by the following formula (1).

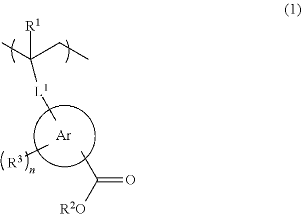

wherein in the formula (1),
R¹ is a hydrogen atom, a fluorine atom, a methyl group, or a trifluoromethyl group;
L¹ is a single bond or a divalent linking group;
Ar represents a furan ring, a thiophene ring, or an aromatic heterocycle having 9 to 20 ring members containing a furan ring or a thiophene ring;
R² is a hydrogen atom or a monovalent organic group having 1 to 20 carbon atoms;
R³ is a halogen atom, a hydroxy group, a cyano group, a nitro group, an amino group, a thiol group, or a monovalent organic group having 1 to 20 carbon atoms;
n is an integer of 0 to 4; and when n is 2 or more, a plurality of R³s are identical to or different from each other.

Examples of the divalent linking group represented by L¹ include divalent hydrocarbon groups such as alkanediyl groups, cycloalkanediyl groups, alkene diyl groups, and arenediyl groups; divalent heteroatom-containing groups such as —CO—, —C(═O)O—, —CS—, —O—, —S—, —SO—, —SO₂—, —NR′—, or a combination of two or more thereof; or a group containing the above divalent heteroatom-containing group between adjacent carbon atoms of the above divalent hydrocarbon group; and combinations thereof. R′ is a hydrogen atom or a monovalent hydrocarbon group having 1 to 10 carbon atoms. Some or all of hydrogen atoms of these groups may be substituted with, for example, a substituent such as a halogen atom such as a fluorine atom, a chlorine atom, a bromine atom, or an iodine atom; a hydroxy group; a carboxy group; a cyano group; a nitro group; an alkyl group; an alkoxy group; an alkoxycarbonyl group; an alkoxycarbonyloxy group; an acyl group; an acyloxy group, or a group obtained by substituting a hydrogen atom of such a group with a halogen atom.

Examples of the alkanediyl group include: alkanediyl groups having 1 to 8 carbon atoms such as a methanediyl group, an ethanediyl group, a 1, 3-propanediyl group, and a 2,2-propanediyl group.

Examples of the cycloalkanediyl group include: monocyclic cycloalkanediyl groups such as a cyclopentanediyl group, and a cyclohexanediyl group; polycyclic cycloalkanediyl groups such as a norbornanediyl group and an adamantanediyl group. A cycloalkanediyl group having 5 to 12 carbon atoms is preferable as the cycloalkanediyl group.

Examples of the alkenediyl groups include: an ethenediyl group, a propenediyl group, and a butenediyl group. An alkenediyl group having 2 to 6 carbon atoms is preferable as the alkenediyl groups.

Examples of the arenediyl groups include: a benzenediyl group, a toluenediyl group, and a naphthalenediyl group. An arenediyl group having 6 to 15 carbon atoms is preferable as the arenediyl groups.

As L¹, a single bond, *—COO—, *—CONH—, or a group formed by combining *—COO— with an alkanediyl group is preferable, and a single bond or *—COO— is more preferable. * is a bond to a carbon atom bonded to R¹.

Examples of the aromatic heterocycle having 9 to 20 ring members containing a furan ring or a thiophene ring represented by Ar include a 1-benzofuran ring, a 2-benzofuran ring, a dibenzofuran ring, a 1-benzothiophene ring, a 2-benzothiophene ring, and a dibenzothiophene ring. The Ar is preferably a furan ring, a thiophene ring, a 1-benzofuran ring, a 2-benzofuran ring, a 1-benzothiophene ring, or a 2-benzothiophene ring, more preferably a furan ring or a thiophene ring.

Examples of the monovalent organic group having 1 to 20 carbon atoms represented by R² include a monovalent hydrocarbon group having 1 to 20 carbon atoms, a group containing between carbon atoms of the hydrocarbon group (between adjacent or non-adjacent two carbon atoms) a divalent hetero atom-containing group, a group obtained by substituting some or all of the hydrogen atoms of the hydrocarbon group with a monovalent hetero atom-containing group, and a group obtained by combining them. The “organic group” refers to a group having at least one carbon atom.

Examples of the monovalent hydrocarbon group having 1 to 20 carbon atoms include a monovalent chain hydrocarbon group having 1 to 20 carbon atoms, a monovalent alicyclic hydrocarbon group having 3 to 20 carbon atoms, or a monovalent aromatic hydrocarbon group having 6 to 20 carbon atoms.

Examples of the monovalent chain hydrocarbon group having 1 to 20 carbon atoms include alkyl groups such as a methyl group, ethyl group, n-propyl group, i-propyl group, n-butyl group, sec-butyl group, iso-butyl group, and tert-butyl group; alkenyl groups such as an ethenyl group, propenyl group, and butenyl group; and alkynyl groups such as an ethynyl group, propynyl group, and butynyl group.

Examples of the monovalent alicyclic hydrocarbon group having 3 to 20 carbon atoms include monocyclic alicyclic saturated hydrocarbon groups such as a cyclopentyl group and cyclohexyl group; polycyclic alicyclic saturated hydrocarbon groups such as a norbornyl group, adamantyl group, tricyclodecyl group, and tetracyclododecyl group; monocyclic alicyclic unsaturated hydrocarbon groups such as a cyclopentenyl group and cyclohexenyl group; and polycyclic alicyclic unsaturated hydrocarbon groups such as a norbornenyl group, tricyclodecenyl group, and tetracyclododecenyl group.

Examples of the monovalent aromatic hydrocarbon group having 6 to 20 carbon atoms include aryl groups such as a phenyl group, tolyl group, xylyl group, naphthyl group, and anthryl group; and aralkyl groups such as a benzyl group, phenethyl group, naphthylmethyl group, and anthrylmethyl group.

Examples of the heteroatom constituting the divalent or monovalent heteroatom-containing group include an oxygen atom, a nitrogen atom, a sulfur atom, a phosphorus atom, a silicon atom, a halogen atom and the like. Examples of the halogen atom include a fluorine atom, a chlorine atom, a bromine atom, and an iodine atom.

As the divalent heteroatom-containing group, for example, the divalent heteroatom-containing groups in the L¹ can be suitably employed.

Examples of the monovalent heteroatom-containing group include a hydroxy group, a carboxy group, a sulfanyl group, a cyano group, a nitro group, an alkoxy group, a halogen atom, and the like.

Examples of the monovalent organic group having 1 to 20 carbon atoms represented by R³ include a monovalent hydrocarbon group having 1 to 20 carbon atoms, a group having between carbon atoms (between two adjacent or non-adjacent carbon atoms) of the hydrocarbon group or at an Ar bond terminal a divalent heteroatom-containing group, a group obtained by substituting some or all of hydrogen atoms of the hydrocarbon group with a monovalent heteroatom-containing group, an amide group, and a group obtained by combining them.

As the monovalent hydrocarbon group having 1 to 20 carbon atoms, the divalent heteroatom-containing group, and the monovalent heteroatom-containing group, a monovalent hydrocarbon group having 1 to 20 carbon atoms, a divalent heteroatom-containing group, and a monovalent heteroatom-containing group in R² can be suitably employed.
n is an integer of 0 to 4, preferably 0 or 1, and more preferably 0.

The monovalent organic group having 1 to 20 carbon atoms represented by R² in the formula (1) is preferably a group represented by the following formula (2).

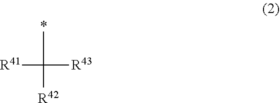

wherein in the formula (2),
R⁴¹ is a hydrogen atom or a monovalent organic group having 1 to 10 carbon atoms;
R⁴² and R⁴³ each independently represent a hydrogen atom or a monovalent organic group having 1 to 10 carbon atoms, or a divalent alicyclic group or heterocyclic group having 3 to 10 ring members composed of R⁴² and R⁴³ combined with each other together with a carbon atom to which R⁴² and R⁴³ are bonded; and
* represents a bond bonded to an oxygen atom in the formula (1).

As the monovalent organic group having 1 to 10 carbon atoms represented by R⁴¹ to R⁴³, a monovalent organic group having 1 to 10 carbon atoms among monovalent organic groups having 1 to 20 carbon atoms represented by R² in the formula (1) can be suitably employed.

As the divalent alicyclic group having 3 to 10 ring members composed of R⁴² and R⁴³ combined with each other together with a carbon atom to which R⁴² and R⁴³ are bonded, a group obtained by removing one hydrogen atom from the monovalent alicyclic hydrocarbon group having 3 to 20 carbon atoms in R² in the formula (1) can be suitably employed. In addition, as the divalent heterocyclic group having 3 to 10 ring members, a group obtained by removing two hydrogen atoms from the following heterocyclic structure can be suitably employed.

Examples of the heterocyclic structure include:
oxygen atom-containing alicyclic heterocyclic structures such as oxirane, tetrahydrofuran, tetrahydropyran, dioxolane, and dioxane;
nitrogen atom-containing alicyclic heterocyclic structures such as aziridine, pyrrolidine, piperidine, and piperazine;
sulfur atom-containing alicyclic heterocyclic structures such as thietane, thiolane, and thiane;
alicyclic heterocyclic structures containing two or more kinds of heteroatoms such as morpholine, 1,2-oxathiolane, and 1, 3-oxathiolane;
oxygen atom-containing aromatic heterocyclic structures such as furan and benzofuran;
nitrogen atom-containing aromatic heterocyclic structures such as pyrrole, pyrazole, and triazine;
sulfur atom-containing aromatic heterocyclic structures such as thiophene; and
aromatic heterocyclic structures containing two or more kinds of heteroatoms such as oxazole, isothiazole, and thiazine. The heterocyclic structures include lactone structures, cyclic carbonate structures, sultone structures, cyclic acetal structures, and a combination thereof.
R⁴¹ is preferably a hydrogen atom, a chain hydrocarbon group having 1 to 5 carbon atoms, an aromatic hydrocarbon group having 6 to 10 carbon atoms, or an alicyclic saturated hydrocarbon group having a divalent heteroatom-containing group between carbon atoms.

It is preferable that R⁴² and R⁴³ be each independently a hydrogen atom, a monovalent chain hydrocarbon group having 1 to 5 carbon atoms, or a divalent alicyclic group or heterocyclic group having 3 to 10 carbon atoms composed of R⁴² and R⁴³ combined with each other together with a carbon atom to which R⁴² and R⁴³ are bonded.

Preferably, examples of the group represented by the above formula (2) include structures represented by the following formulas (2-1-1) to (2-1-5).

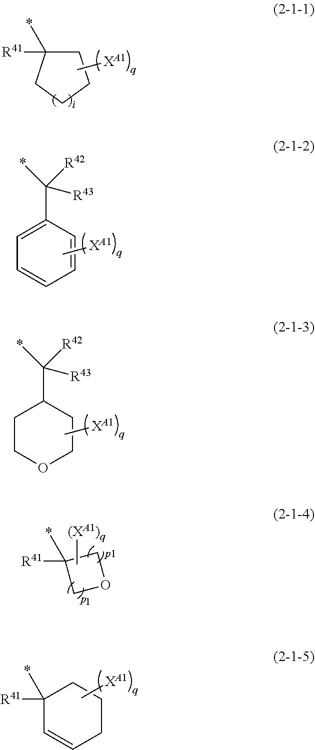

In the formulas (2-1-1) to (2-1-5), R⁴¹ to R⁴³ have the same meaning as in the formula (2). i is an integer of 1 to 4. X^{A1} is a substituent, and as the substituent, those exemplified as the substituent that can be possessed by the divalent linking group represented by L¹ in the formula (1) can be suitably employed. q is an integer of 0 to 2. p1 is an integer of 1 to 3. * is a bond to an oxygen atom.

Examples of the monomers that afford the structural unit (I) include, but are not limited to, those shown below. In the following formulas, R¹ has the same meaning as in the above formula (1).

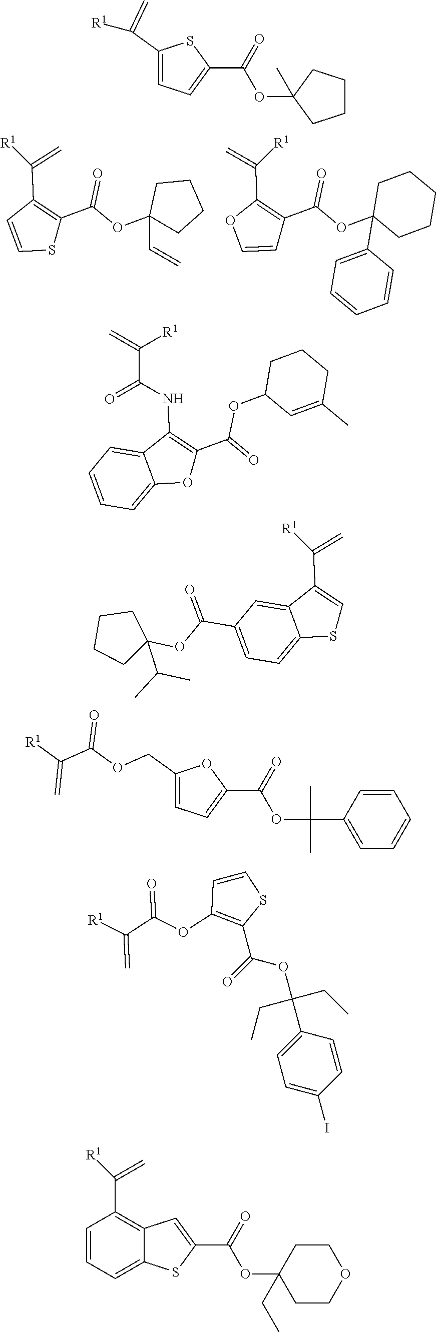

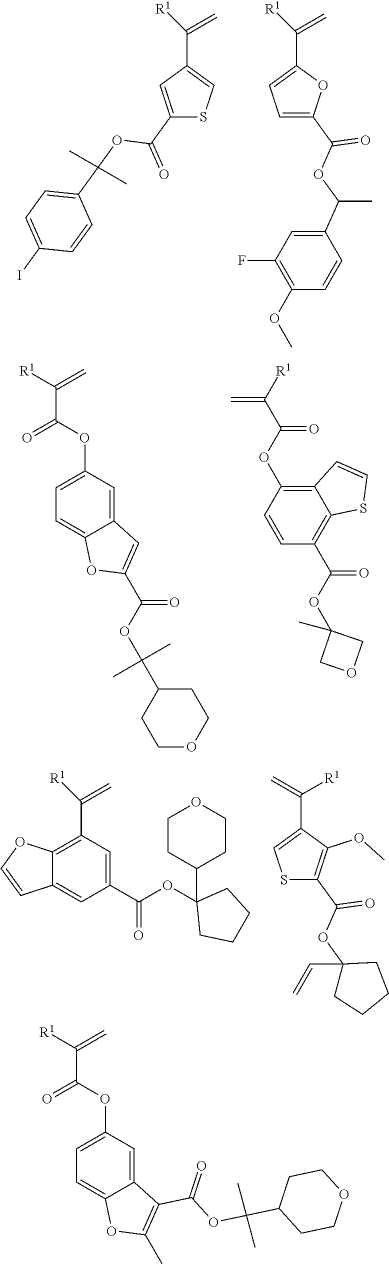

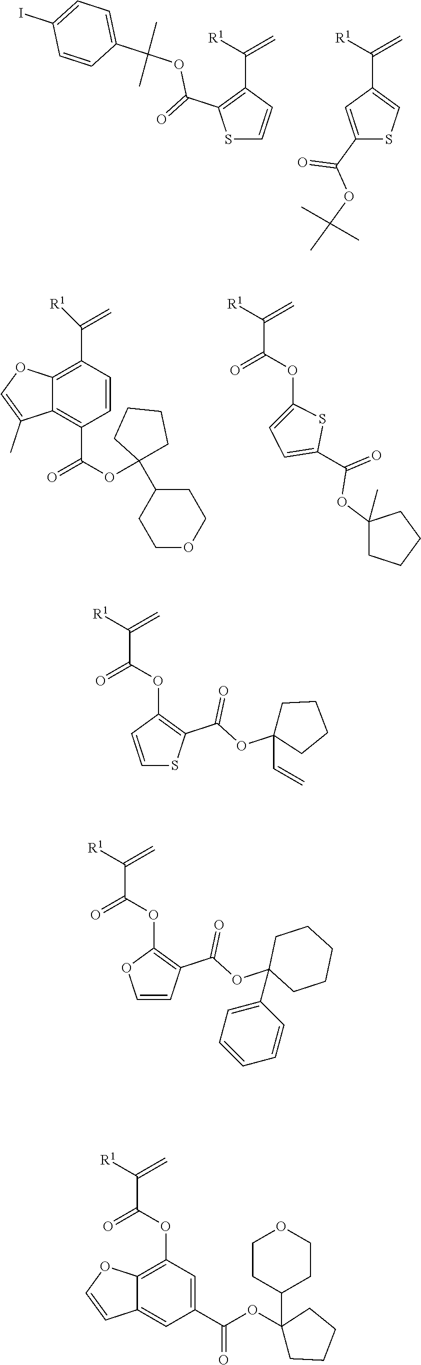

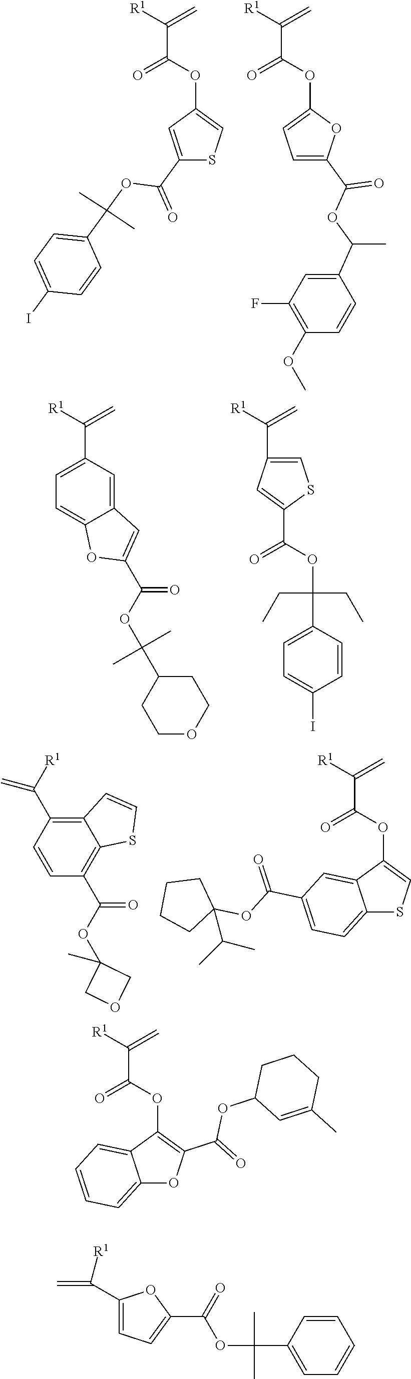

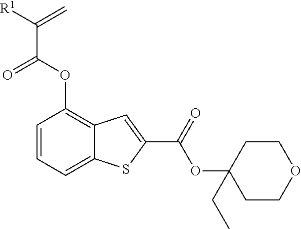

The structural unit (I) preferably contains an iodo group from the viewpoint of sensitivity.

The structural unit (I) may be a structural unit derived from at least one of Compounds 1 to 5 described below.

The base polymer (A) may contain one type or two or more types of the structural unit (I).

The upper limit of the content of the structural unit (I) (when a plurality of types is contained, the total content thereof is taken) is preferably 60 mol %, more preferably 50 mol, still more preferably 40 mol %, and particularly preferably 35 mol % based on all structural units composing the base polymer (A) (or the base polymer (A1)). The lower limit of the content is not particularly limited, but is, for example, preferably 1 mol %, more preferably 5 mol %, still more preferably 10 mol % and particularly preferably 15 mol %. When the contents of the structural unit (I) are adjusted to within the above range, it is preferable from the viewpoints of sensitivity, CDU, and development defects of the radiation-sensitive composition.

### (Structural Unit (II))

Preferably, the polymer (A) (or the polymer (A1)) further contains a structural unit (II) having a phenolic hydroxy group. Examples of the monomer that affords the structural unit (II) include, but are not limited to, those shown below. In the following formulas, R^{A} is a hydrogen atom, a fluorine atom, a methyl group, or a trifluoromethyl group.

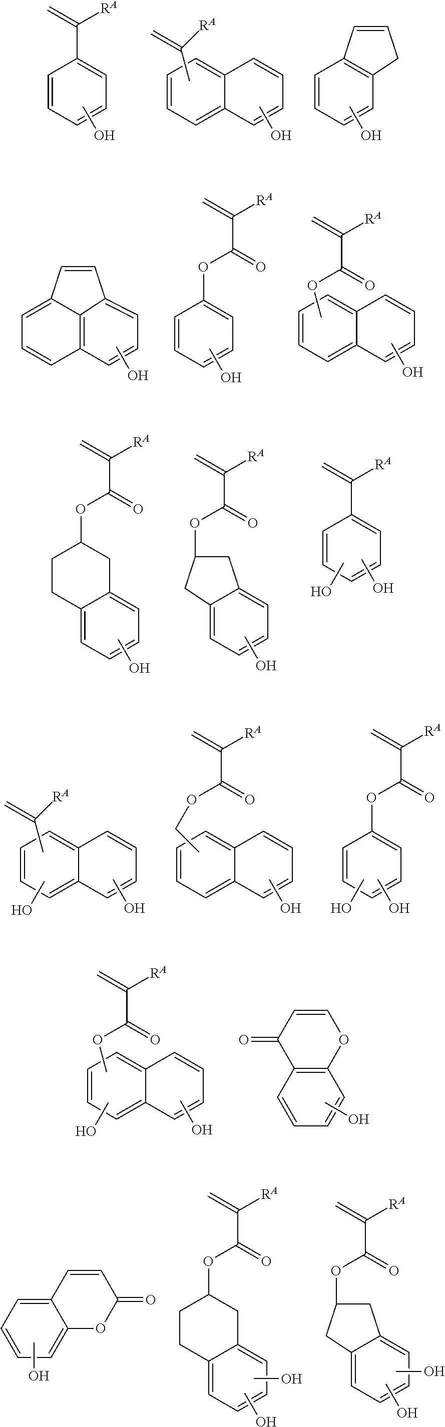

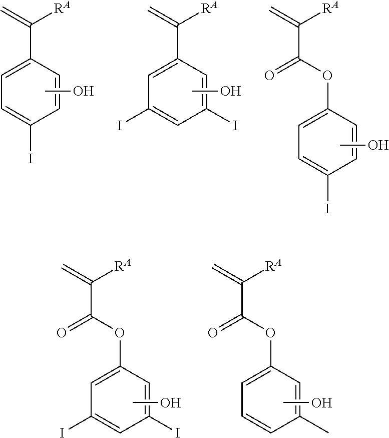

When the polymer (A) (or the polymer (A1)) has the structural unit (II), the lower limit of the content of the structural unit (II) (when a plurality of types is contained, the total content thereof is taken) is preferably 15 mol %, more preferably 20 mol %, still more preferably 25 mol % and particularly preferably 30 mol % based on all structural units composing the base polymer (A) (or base polymer (A1)). The upper limit of the content is preferably 70 mol %, more preferably 60 mol %, still more preferably 55 mol % and particularly preferably 50 mol %. When the content of the structural unit (II) is adjusted to within the above range, the patternability of the radiation-sensitive composition can be further improved.

### (Structural Unit (III))

The polymer (A) (or the polymer (A1)) can further contain a structural unit (III) having an acid-dissociable group (provided that the structural unit (I) is excluded). The structural unit (III) is not particularly limited as long as the structural unit (III) contains an acid-dissociable group, and examples thereof include a structural unit having a tertiary alkyl ester moiety, a structural unit having a structure in which a hydrogen atom of a phenolic hydroxy group is substituted with a tertiary alkyl group, and a structural unit having an acetal linkage.

The “acid-dissociable group” refers to a group that substitutes for a hydrogen atom of a carboxy group, a phenolic hydroxyl group, an alcoholic hydroxyl group, a sulfo group, or the like, and is dissociated by the action of an acid. The acid generated from the radiation-sensitive acid-generating polymer (A1) or the radiation-sensitive acid generator (C) through exposure to light dissociates the acid-dissociable group in the structural unit (III) to generate a carboxy group or the like. As a result, a difference in solubility into a developer arises between the exposed portion and the unexposed portion of a resist film, making it possible to achieve pattern formation.

As the structural unit (III) having an acid-dissociable group, from the viewpoint of improving the pattern-forming performance of the radiation-sensitive composition, a structural unit represented by the following formula (4) (hereinafter also referred to as “structural unit (III-1)”) is preferred.

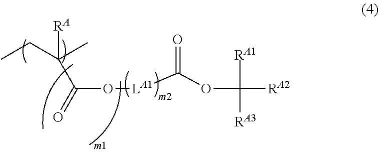

In the above formula (4), R^{A} is a hydrogen atom, a fluorine atom, a methyl group, or a trifluoromethyl group. RAL is a hydrogen atom or a monovalent organic group having 1 to 20 carbon atoms. R^{A2} and R^{A3} each independently are a monovalent chain hydrocarbon group having 1 to 20 carbon atoms, or a monovalent alicyclic hydrocarbon group having 3 to 20 carbon atoms, or represent a divalent alicyclic group or heterocyclic group having 3 to 20 ring members composed of these groups combined with each other together with a carbon atom to which these groups are bonded. m1 and m2 are each independently 0 or 1, provided that when m1 is 1, m2 is 1. L^{A1} represents a divalent linking group.

In the monovalent organic group having 1 to 20 carbon atoms represented by R^{A1}, the monovalent chain hydrocarbon group having 1 to 20 carbon atoms represented by R^{A2} and R^{A3}, and the monovalent alicyclic hydrocarbon group having 3 to 20 carbon atoms, some or all of hydrogen atoms on the carbon atoms may be substituted with a substituent. As the substituent, those exemplified as the substituent that can be possessed by the divalent linking group represented by L¹ in the formula (1) can be suitably employed.

Examples of the divalent linking group represented by LAI include divalent hydrocarbon groups such as an alkanediyl group, a cycloalkanediyl group, an alkenediyl group, and an arenediyl group; and groups containing, between carbon-carbon bonds of the above divalent hydrocarbon group, a divalent heteroatom-containing group selected from —CO—, —C(═O)O—, —OC(═O)—, —CS—, —O—, —S—, —SO—, —SO₂—, and —NR′—. R′ is a hydrogen atom or a monovalent hydrocarbon group having 1 to 10 carbon atoms. Some or all of hydrogen atoms of these groups may be substituted with, for example, a substituent such as a halogen atom such as a fluorine atom, a chlorine atom, a bromine atom, or an iodine atom; a hydroxy group; a carboxy group; a cyano group; a nitro group; an alkyl group; an alkoxy group; an alkoxycarbonyl group; an alkoxycarbonyloxy group; an acyl group; an acyloxy group, or a group obtained by substituting a hydrogen atom of such a group with a halogen atom.

As the monovalent organic group having 1 to 20 carbon atoms represented by R^{A1}, the monovalent organic group having 1 to 20 carbon atoms represented by R² in the above formula (1) can be suitably employed.

As the monovalent chain hydrocarbon group having 1 to 20 carbon atoms and the monovalent alicyclic hydrocarbon group having 3 to 20 carbon atoms represented by R^{A2} and R^{A3}, the monovalent chain hydrocarbon group having 1 to 20 carbon atoms and the monovalent alicyclic hydrocarbon group having 3 to 20 carbon atoms represented by R² in the formula (1) can be suitably employed.

As the divalent alicyclic group and heterocyclic group having 3 to 20 ring members composed of R^{A2} and R^{A3} combined with each other together with a carbon atom to which R^{A2} and R^{A3} are bonded, a divalent alicyclic group or heterocyclic group having 3 to 10 carbon atoms composed of R⁴² and R⁴³ in the formula (2) combined with each other together with a carbon atom to which R⁴² and R⁴³ are bonded can be suitably employed.

R^{A1} is preferably a hydrogen atom, a linear or branched saturated hydrocarbon group having 1 to 20 carbon atoms, a monovalent aromatic hydrocarbon group having 6 to 20 carbon atoms, or a monovalent heterocyclic group having 5 to 20 carbon atoms, more preferably a hydrogen atom, an alkyl group having 1 to 4 carbon atoms, a substituted or unsubstituted tetrahydrofuranyl group, or a substituted or unsubstituted phenyl group.

R^{A2} and R^{A3} are each independently preferably a monovalent chain hydrocarbon group having 1 to 10 carbon atoms, or a divalent alicyclic group or heterocyclic group having 3 to 20 carbon atoms in which R^{A2} and R^{A3} are combined with each other and configured together with a carbon atom to which R^{A2} and R^{A3} are bonded, more preferably an alkyl group having 1 to 4 carbon atoms, or a cycloalkanediyl group, a cycloalkenediyl group, a tetrahydrofurandiyl group, or a tetrahydropyrandiyl group in which R^{A2} and R^{A3} are combined with each other and configured together with a carbon atom to which R^{A2} and R^{A3} are bonded.
m1 is preferably 0, m2 is preferably 0, and L^{A1} is preferably an arenediyl group.

Examples of the structural unit (III-1) include structural units represented by the following formulas (4-1) to (4-9).

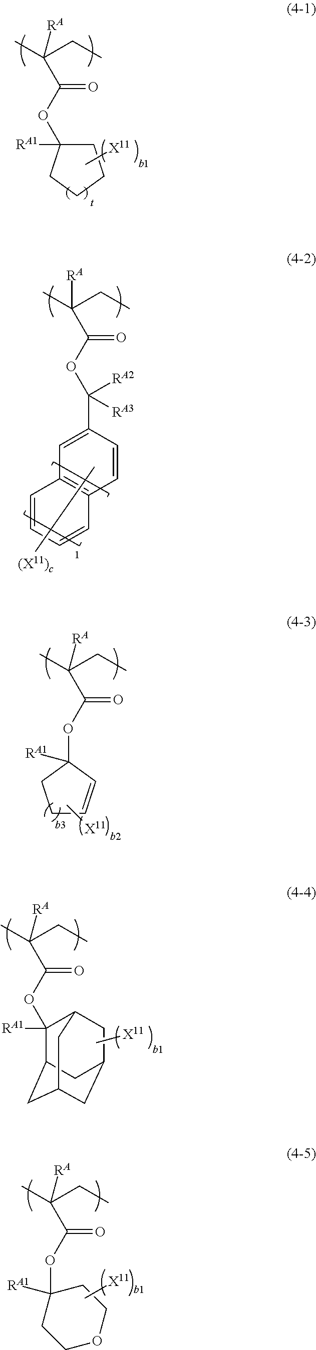

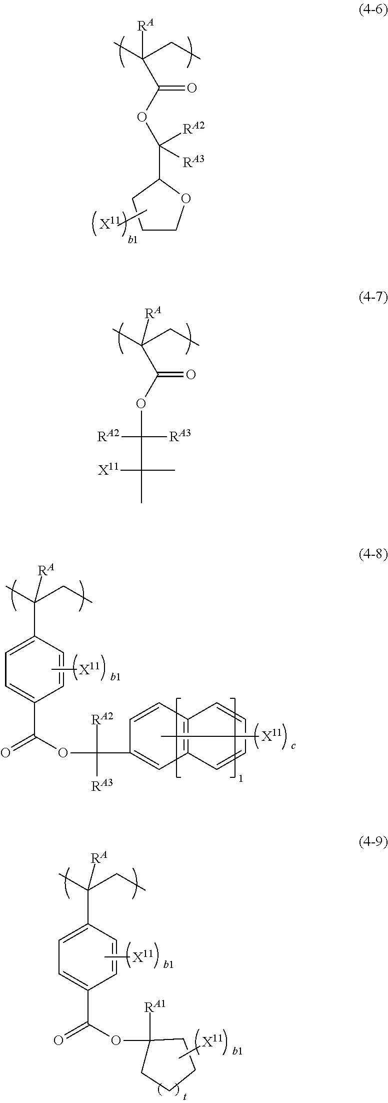

In the above formulas (4-1) to (4-9), R^{A} and RAL to R^{A3} have the same meaning as in the above formula (4). t is an integer of 1 to 4, and 1 is an integer of 0 to 2. c is an integer of 0 to 9 and satisfies 0≤c≤21±5. b1 is an integer of 0 to 3, b2 is an integer of 0 to 10, and b3 is an integer of 1 to 3.

In the formulas (4-1) to (4-9), X¹¹ is a substituent, and as the substituent, those listed as substituents that may be possessed by the divalent linking group represented by L¹ in the above formula (1) can be suitably employed. When b1, c and b2 are 2 or more, a plurality of X¹¹s may be the same as or different from each other.

Specific examples of the structural unit (III) include, but are not particularly limited to, structures represented by the following formulas. Among those shown below, as the structure having an iodine-substituted aromatic ring structure, a structure in which an iodine atom in the following formulas is substituted by an atom or group other than an iodine atom such as a hydrogen atom or another substituent can also be suitably employed.

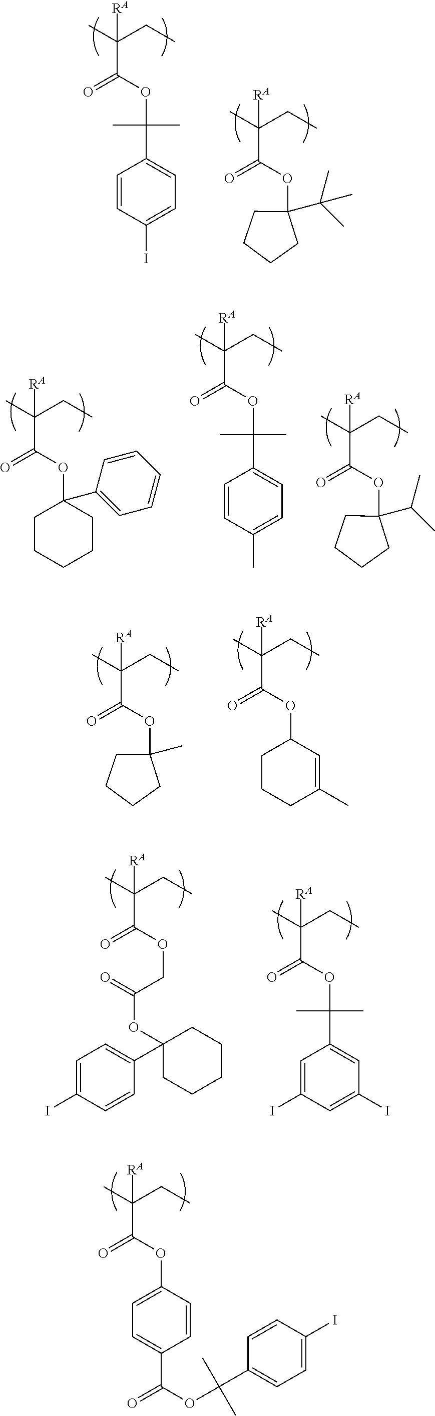

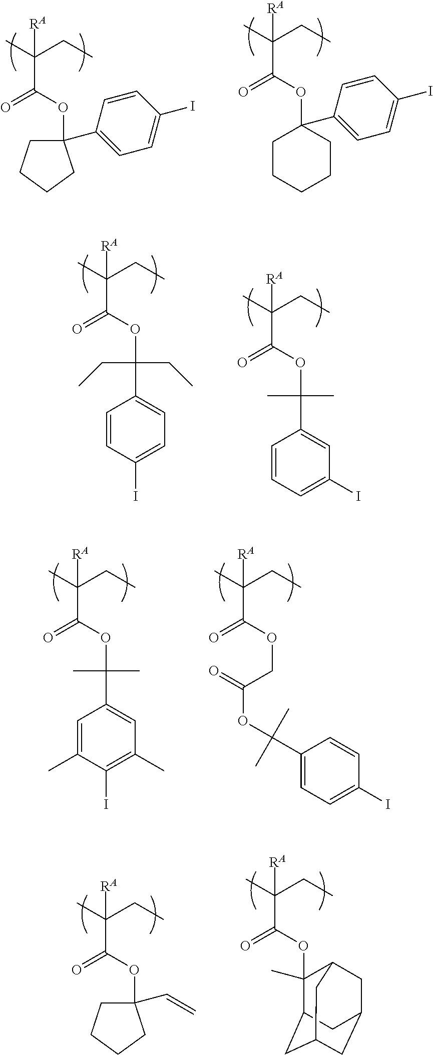

In the formulas, R^{A} has the same meaning as in the formula (4).

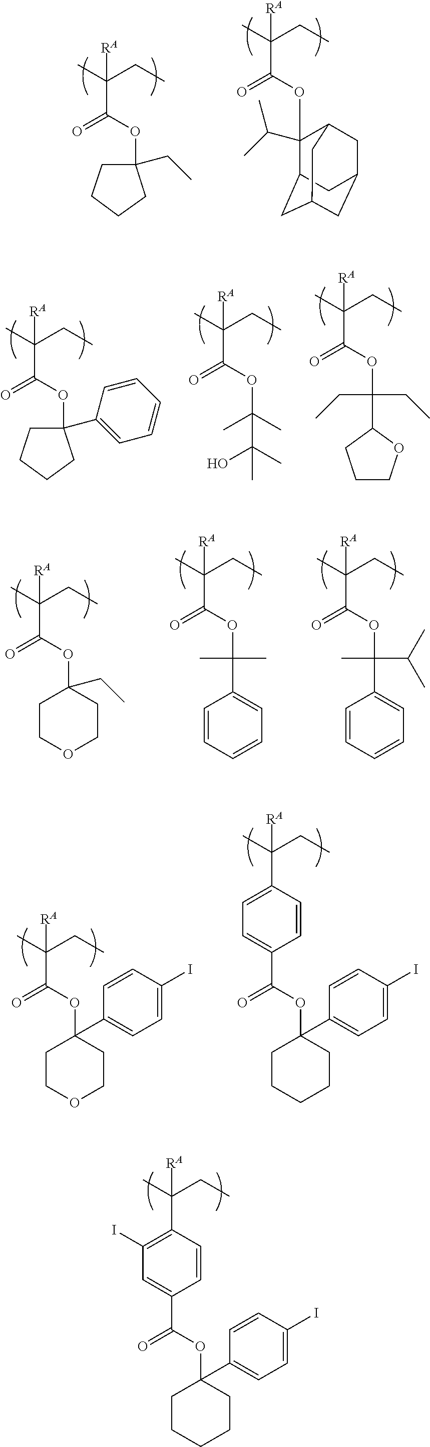

In the formulas, R^{A} has the same meaning as in the formula (4).

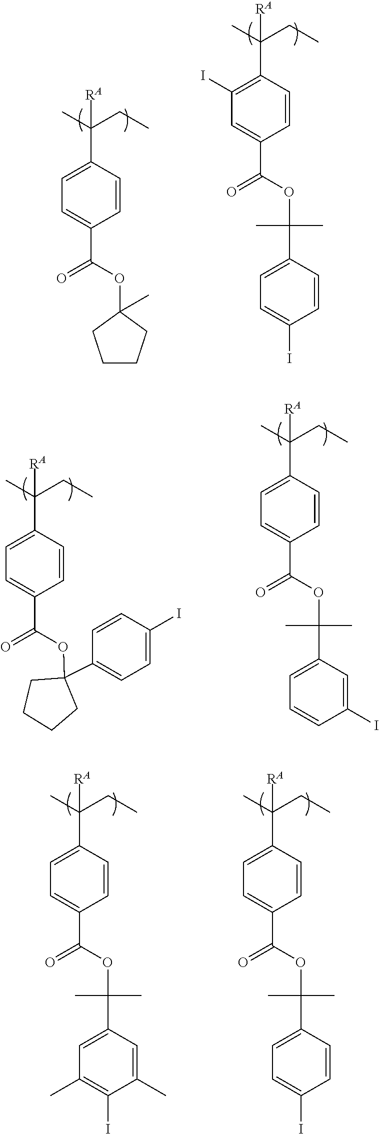

In the formulas, R^{A} has the same meaning as in the formula (4).

The base polymer may contain one type or a combination of two or more types of the structural units (III).

When the polymer (A) (or the radiation-sensitive acid-generating polymer (A1)) has the structural unit (III), the lower limit of the content of the structural unit (III) (when a plurality of types is contained, the total content thereof is taken) is preferably 10 mol %, more preferably 20 mol %, still more preferably 30 mol %, and particularly preferably 35 mol % based on all structural units composing the base polymer (A) (or base polymer (A1)). The upper limit of the content is preferably 70 mol %, more preferably 60 mol %, and still more preferably 50 mol %. When the content of the structural unit (III) is adjusted to within the above range, the patternability of the radiation-sensitive composition can be further improved.

### (Structural Unit (IV))

The polymer (A) may contain a radiation-sensitive acid-generating structural unit (IV). The radiation-sensitive acid-generating structural unit (IV) has a first organic acid anion and a first onium cation.

The form of the first organic acid anion and the first onium cation contained in the structural unit (IV) of the base polymer is not particularly limited, and the base polymer may have the organic acid anion as a side chain portion or may have the onium cation as a side chain portion. Having as a side chain portion means that the corresponding organic acid anion or onium cation is bonded (covalently bonded) to the main chain as a side chain structure of the base polymer. When the organic acid anion is bonded to the main chain as a side chain structure of the base polymer, the onium cation is ionically bonded to the organic acid anion as a counter ion of the organic acid anion. On the other hand, when the onium cation is bonded to the main chain as a side chain structure of the base polymer, the organic acid anion is ionically bonded to the onium cation as a counter ion of the onium cation. From the viewpoint of controlling the acid diffusion length, the base polymer preferably has the organic acid anion as a side chain portion.

Preferably, the first organic acid anion has a sulfonic acid anion, and has an electron withdrawing group such as a fluorine atom or a fluorinated hydrocarbon group bonded to a carbon atom adjacent to the sulfonic acid anion. As a result, the strength of the acid generated through exposure to light can be sufficiently enhanced to a level required for dissociation of the acid-dissociable group.

The structure of the first organic acid anion is not particularly limited, but for example, preferably includes —O—, —CO—, a cyclic structure, or a combination thereof. The combination also includes a structure (heterocyclic structure) in which —O— or —CO— is incorporated as a moiety for forming a ring in a cyclic structure.

The cyclic structure may be any of a monocyclic ring, a polycyclic ring, or a combination thereof. The cyclic structure may be any of an alicyclic structure, an aromatic ring structure, a heterocyclic structure, or a combination thereof. In the case of the combination, the ring structure may be a structure in which ring structures are bonded through a chain structure, or two or more ring structures may form a condensed ring structure, or a bridged ring structure. A divalent heteroatom-containing group may be present between carbons forming the skeleton of the cyclic structure or the chain structure, and some or all of hydrogen atoms on carbon atoms of the cyclic structure or the chain structure may be substituted with another substituent. The first organic acid anion preferably has at least one cyclic structure selected from the group consisting of an alicyclic structure and an aromatic ring structure.

As the alicyclic structure, a structure corresponding to the monovalent alicyclic hydrocarbon group having 3 to 20 carbon atoms in R^{A2} and R^{A3} in the formula (4) can be suitably employed.

The aromatic ring structure is not particularly limited as long as the ring forms a ring structure having aromaticity. Examples of the aromatic ring include aromatic hydrocarbon rings such as a benzene ring, a naphthalene ring, an anthracene ring, a phenalene ring, a phenanthrene ring, a pyrene ring, a fluorene ring, a perylene ring, and a coronene ring; aromatic heterocycles such as a furan ring, a pyrrole ring, a thiophene ring, a phosphole ring, a pyrazole ring, an oxazole ring, an isoxazole ring, a thiazole ring, a pyridine ring, a pyrazine ring, a pyrimidine ring, a pyridazine ring, a triazine ring, a carbazole ring, and a dibenzofuran ring, or combinations thereof.

Examples of the heterocyclic structure include those corresponding to the divalent heterocyclic group having 3 to 10 ring members in R² of the above formula (1).

As the chain structure, monovalent chain hydrocarbon groups having 1 to 20 carbon atoms represented by R^{A2} and R^{A3} in the above formula (4) can be suitably employed.

As the divalent heteroatom-containing group, the divalent heteroatom-containing group in R² of the above formula (1) can be suitably employed.

As the substituent with which some or all of hydrogen atoms on carbon atoms of the cyclic structure or the chain structure are substituted, a substituent of L^{A1} in the formula (4) can be suitably employed.

As the structural unit that affords the above first organic acid anion, a structural unit represented by the following formula (a1) is preferable.

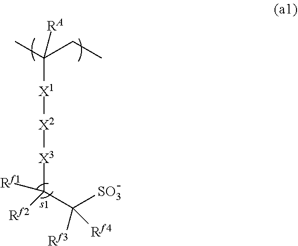

In the formulas, R^{A} is a hydrogen atom or a methyl group. X¹ is a single bond or an ester group. X² is a linear, branched or cyclic alkylene group, having 1 to 12 carbon atoms, a cycloalkylene group having 3 to 12 carbon atoms, an allylene group having 6 to 10 carbon atoms, or a combination thereof, and some of the methylene groups constituting the alkylene group may be replaced by an ether group, an ester group, or a lactone ring-containing group. X³ is a single bond, an ether group, an ester group, a linear or branched alkylene group having 1 to 12 carbon atoms, or a cyclic cycloalkylene group having 3 to 12 carbon atoms, and a part of a methylene group composing the alkylene group may be replaced by an ether group or an ester group. Some or all of the hydrogen atoms of X² and X³ may be replaced by a heteroatom or a monovalent hydrocarbon group having 1 to 20 carbon atoms and optionally containing a heteroatom. It is preferable that at least one of X² and X³ contains an iodine atom, and it is more preferable that X² contains an iodine-substituted aromatic ring structure.

Rf¹ to Rf⁴ are each independently a hydrogen atom, a fluorine atom, or a trifluoromethyl group, and at least one of Rf¹ to Rf⁴ is a fluorine atom or a trifluoromethyl group. s1 is 0 or 1.

As the monovalent hydrocarbon group having 1 to 20 carbon atoms in X² and X³, an alkyl group having 1 to 12 carbon atoms, a cycloalkyl group having 3 to 12 carbon atoms, or an aryl group having 6 to 20 carbon atoms is preferable, and some or all of hydrogen atoms of these groups may be substituted with a heteroatom-containing group such as a hydroxy group, a carboxy group, a halogen atom, an oxo group, a cyano group, an amide group, a nitro group, a sultone group, a sulfone group or a sulfonium salt-containing group, an alkoxy group, or an alkoxycarbonyl group, and a part of a methylene group composing these groups may be substituted with an ether group, an ester group, a carbonyl group, a carbonate group, or a sulfonate ester group.

Preferably, the structural unit that afford the first organic acid anion are each represented by the following formulas (a1-1), (a2-1), (a3-1) and (a4-1).

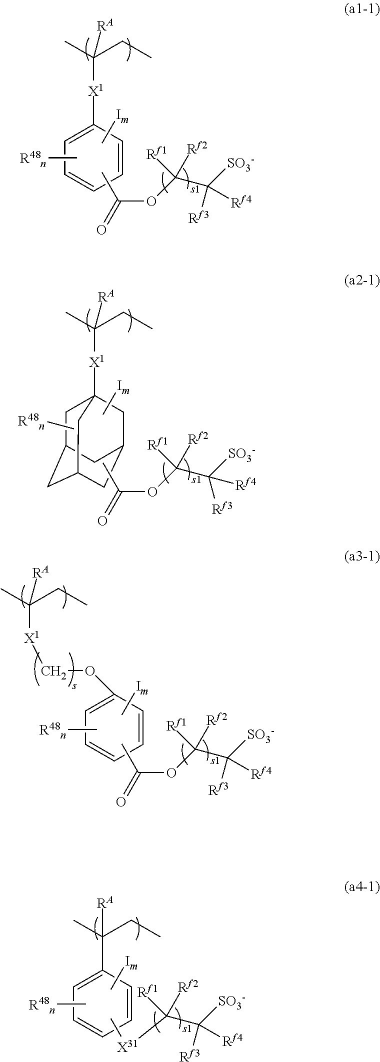

In the formulas, R^{A}, Rf¹ to Rf⁴, X¹, and s1 have the same meanings as in the formula (a1). R⁴⁸ is a linear, branched or cyclic alkyl group having 1 to 4 carbon atoms, a halogen atom other than iodine, a hydroxy group, a linear, branched or cyclic alkoxy group having 1 to 4 carbon atoms, or a linear, branched or cyclic alkoxycarbonyl group having 2 to 5 carbon atoms. In formula (a4-1), x³¹ is a cycloalkylene group having 3 to 12 carbon atoms, and part of the methylene groups constituting the cycloalkylene group may be substituted with an ether group or an ester group. m is an integer of 0 to 4. n is an integer of 0 to 3. It is to be noted 0≤m+n≤4. s is an integer of 1 to 5.

Examples of the monomers that afford the structural unit (IV) include, but are not limited to, those shown below. Among those shown below, as the structure having an iodine-substituted aromatic ring structure, a structure in which an iodine atom in the following formulas is substituted by an atom or group other than an iodine atom such as a hydrogen atom or another substituent can also be suitably employed.

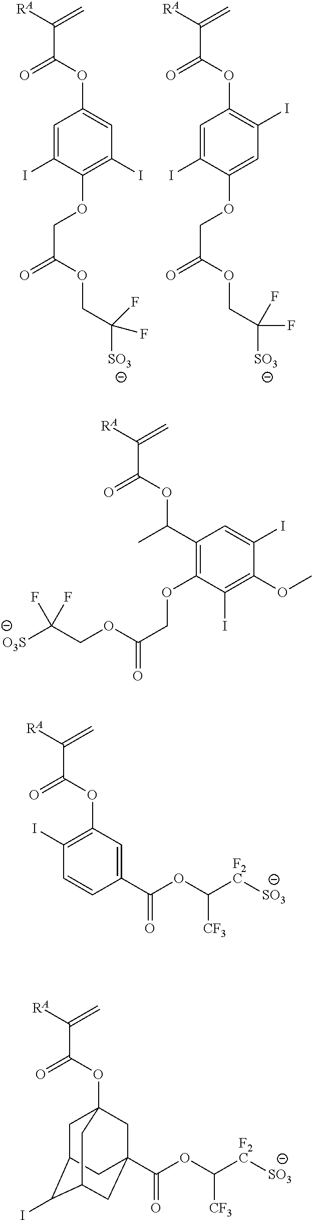

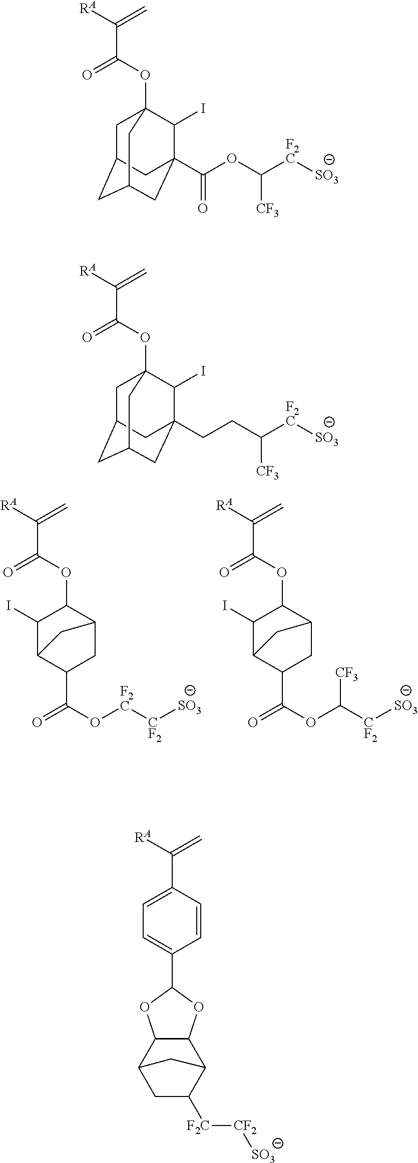

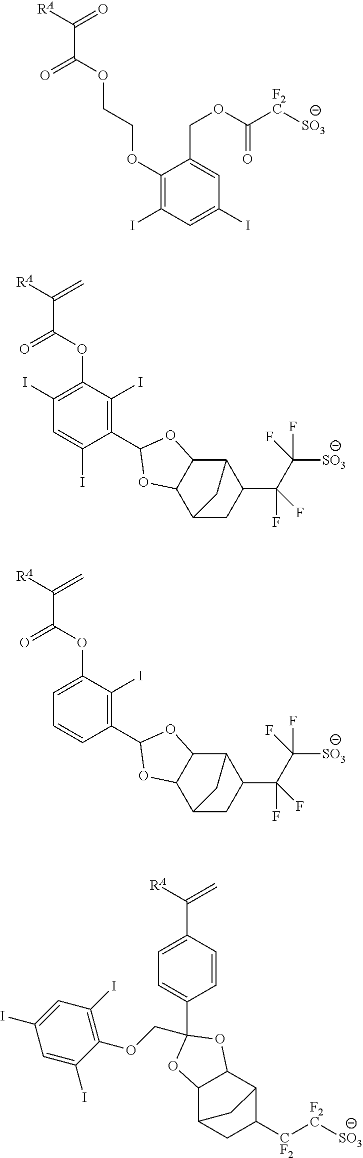

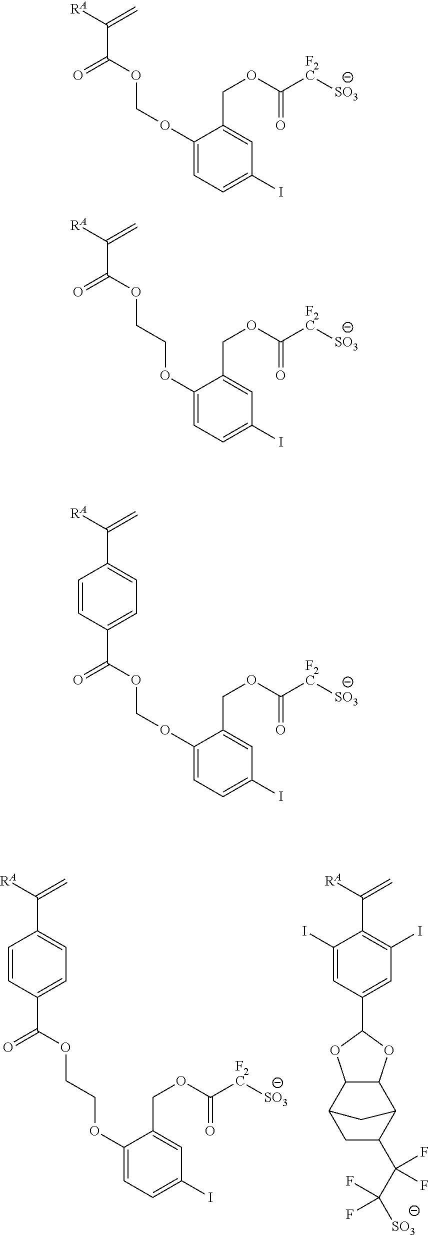

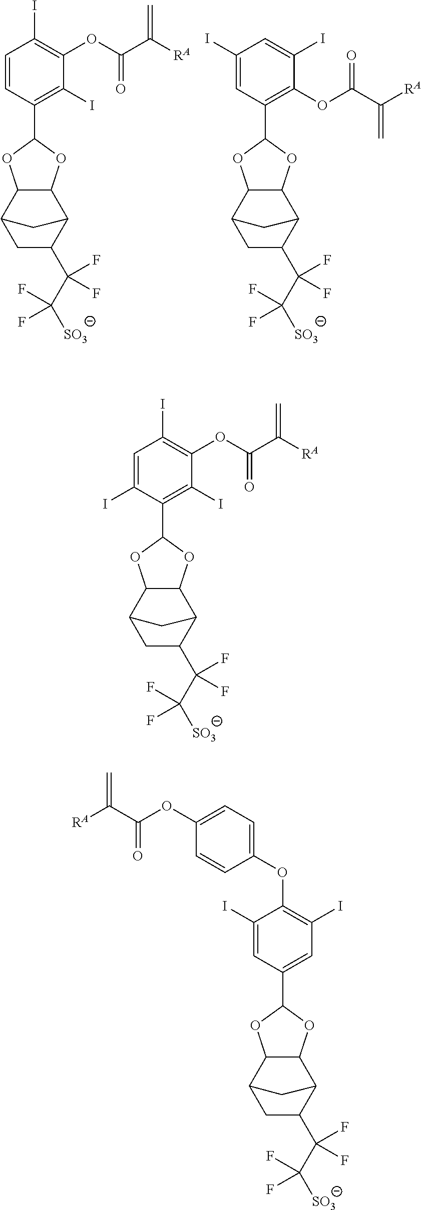

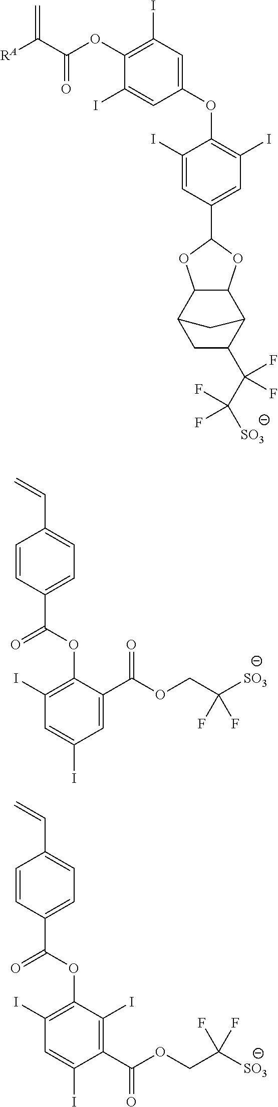

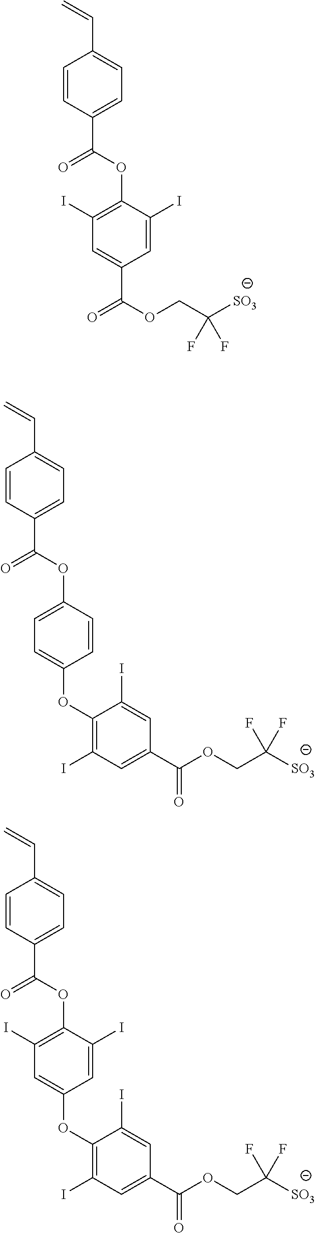

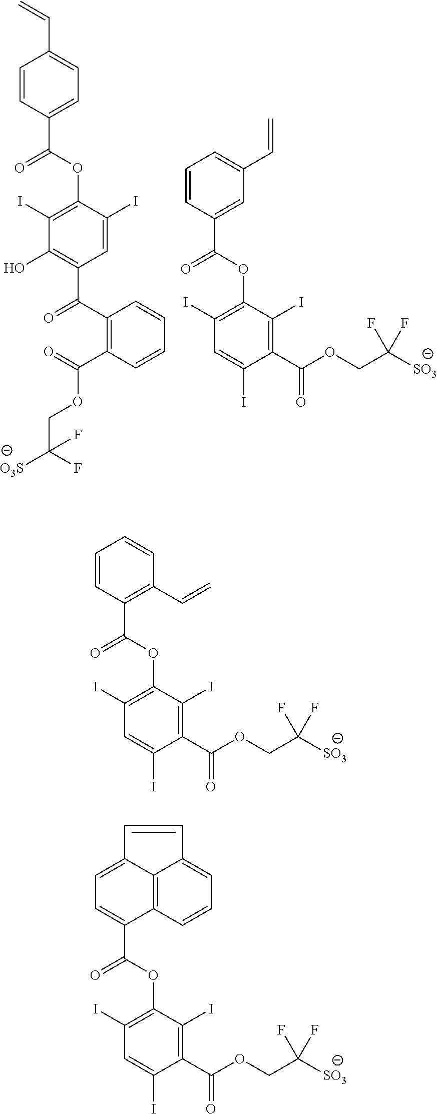

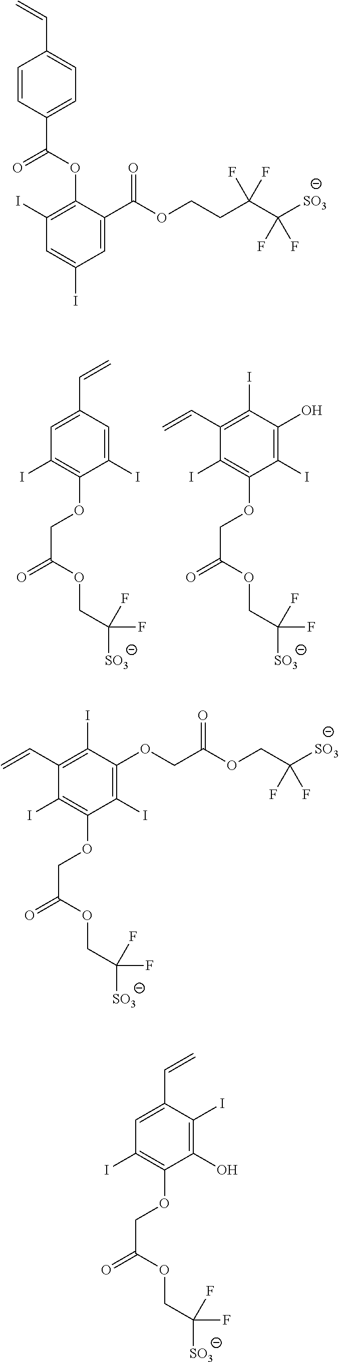

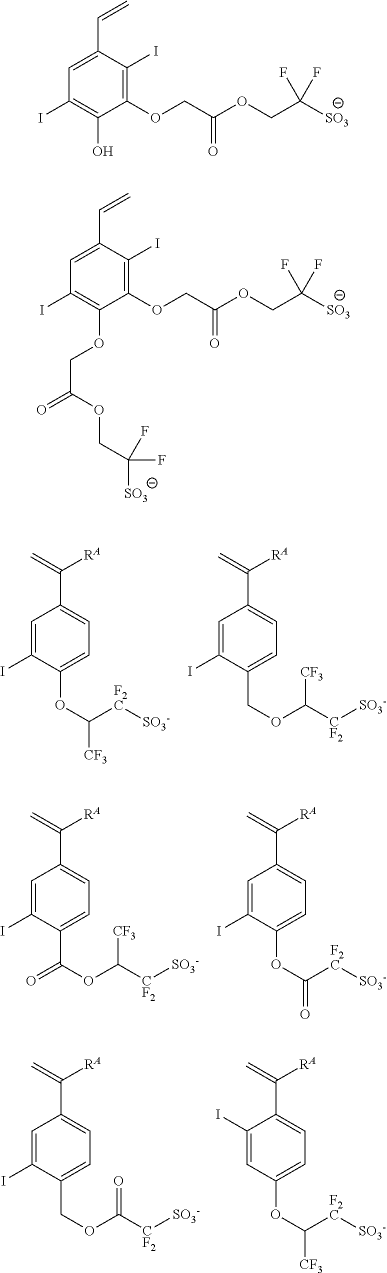

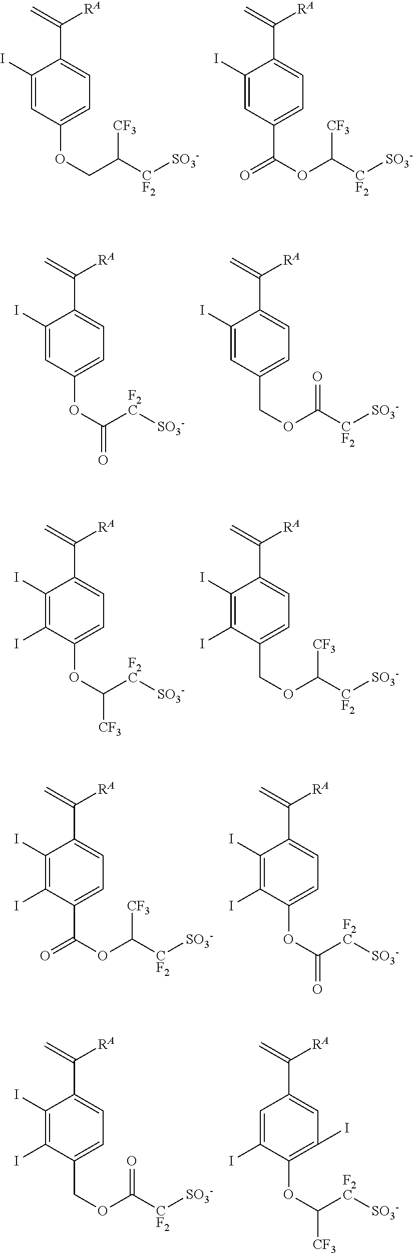

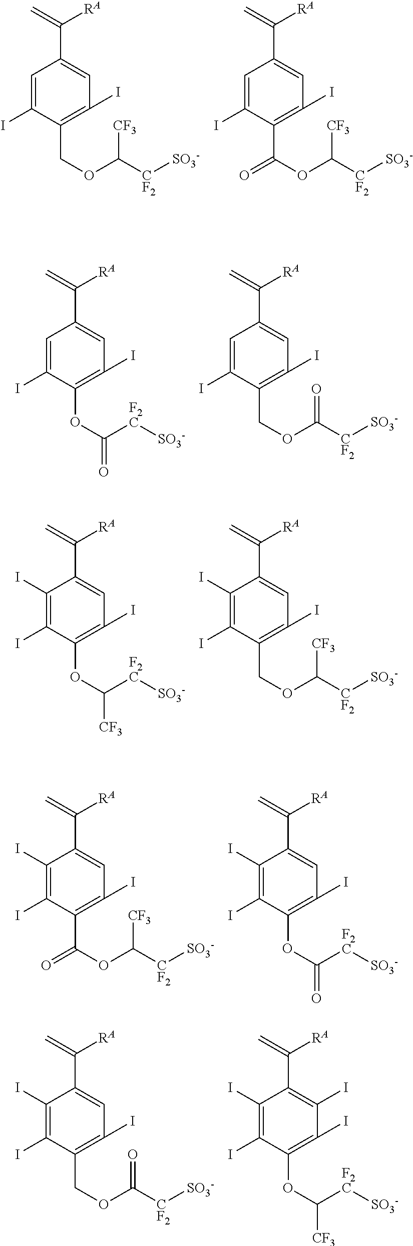

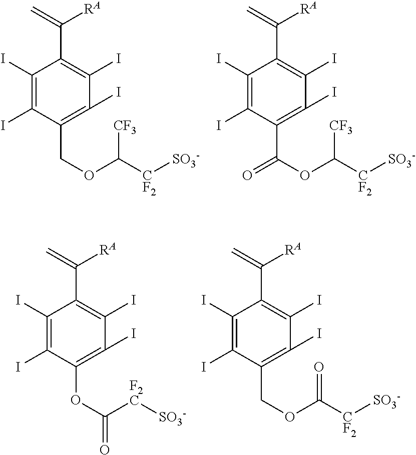

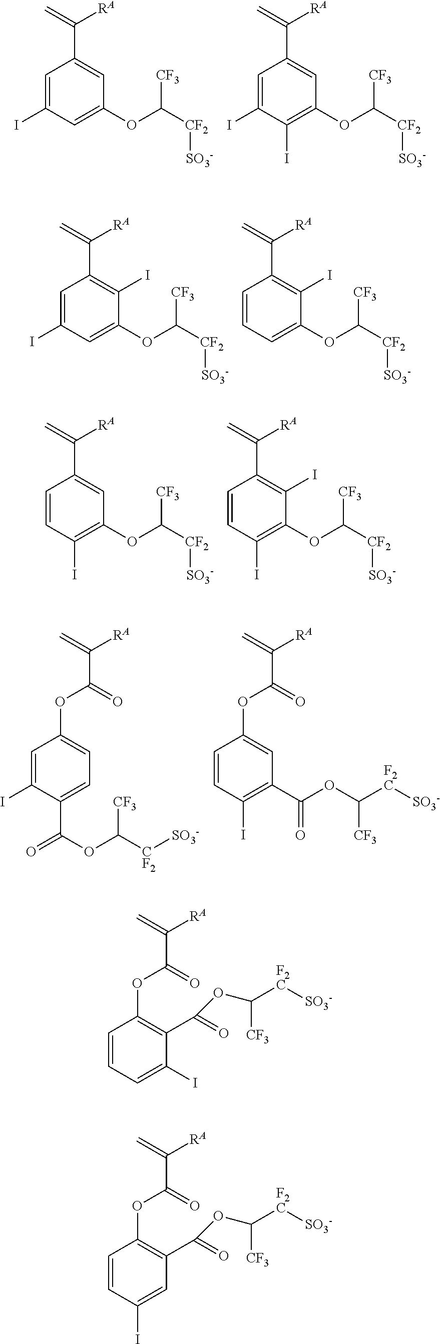

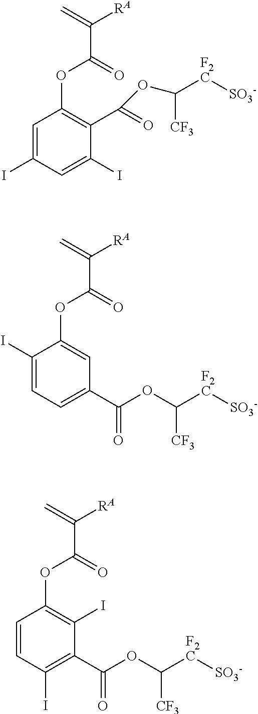

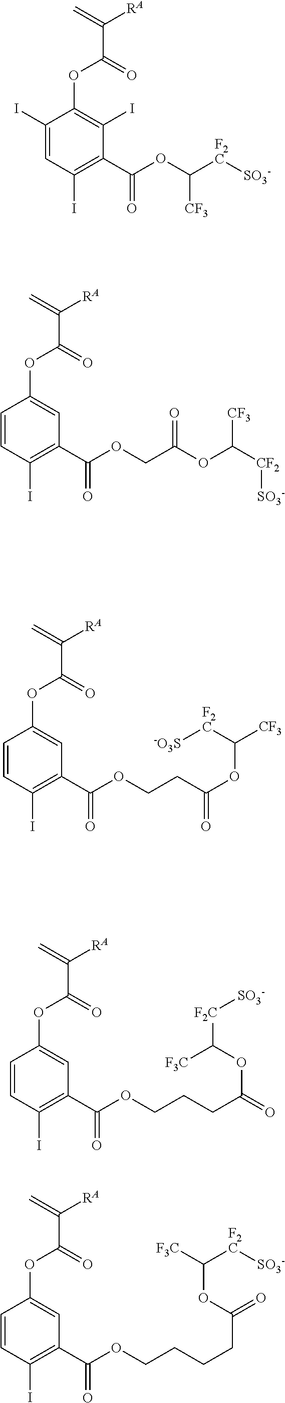

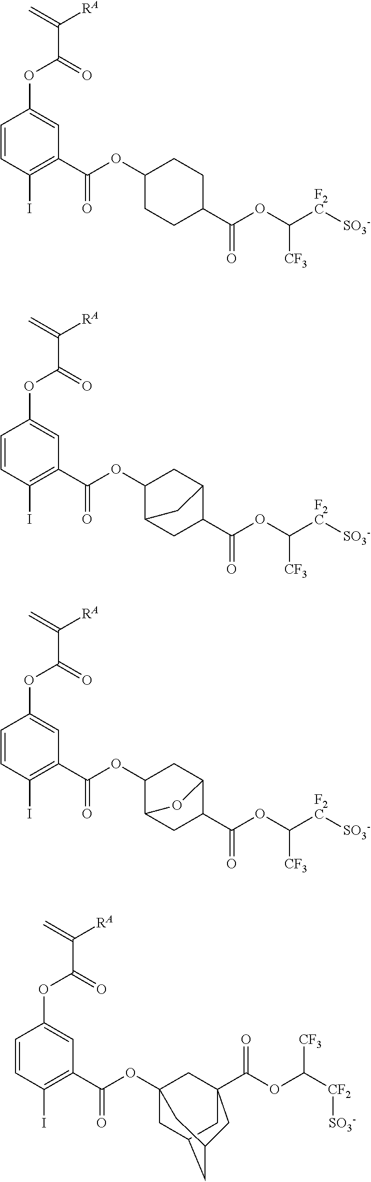

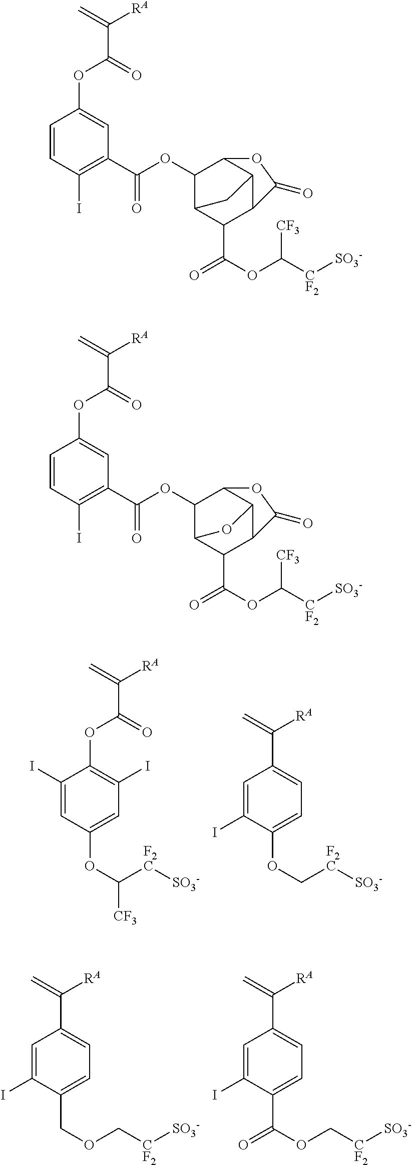

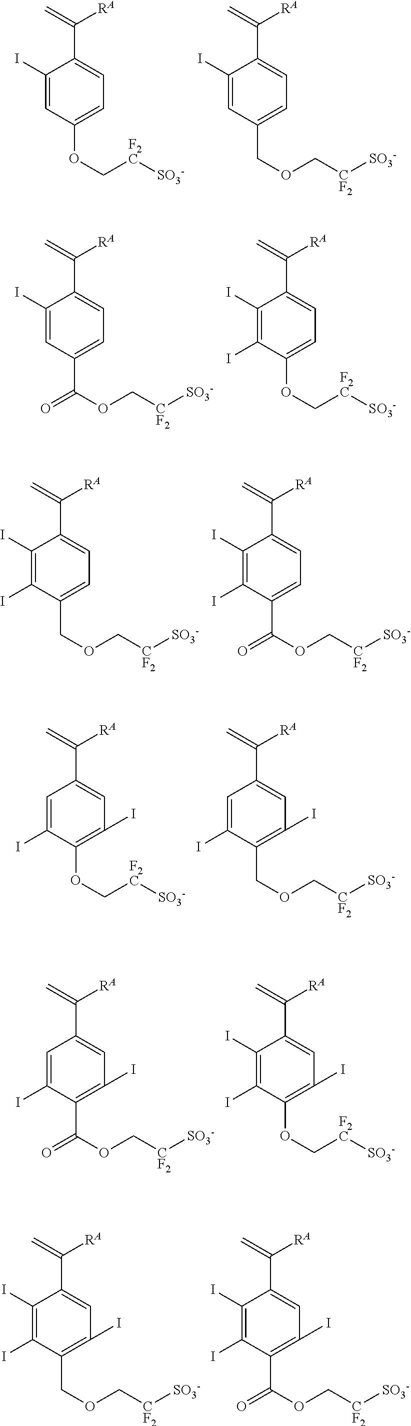

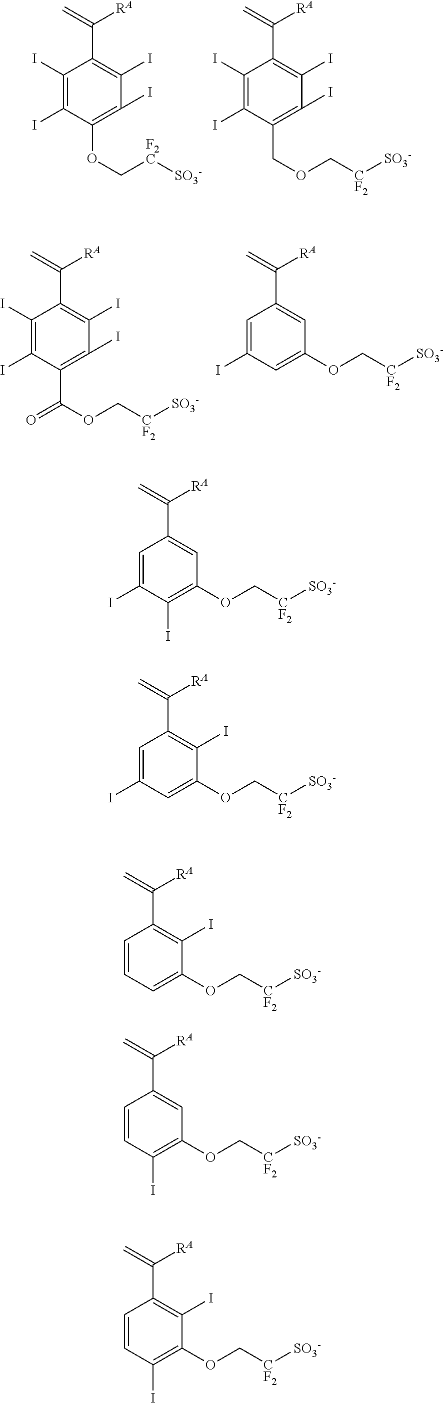

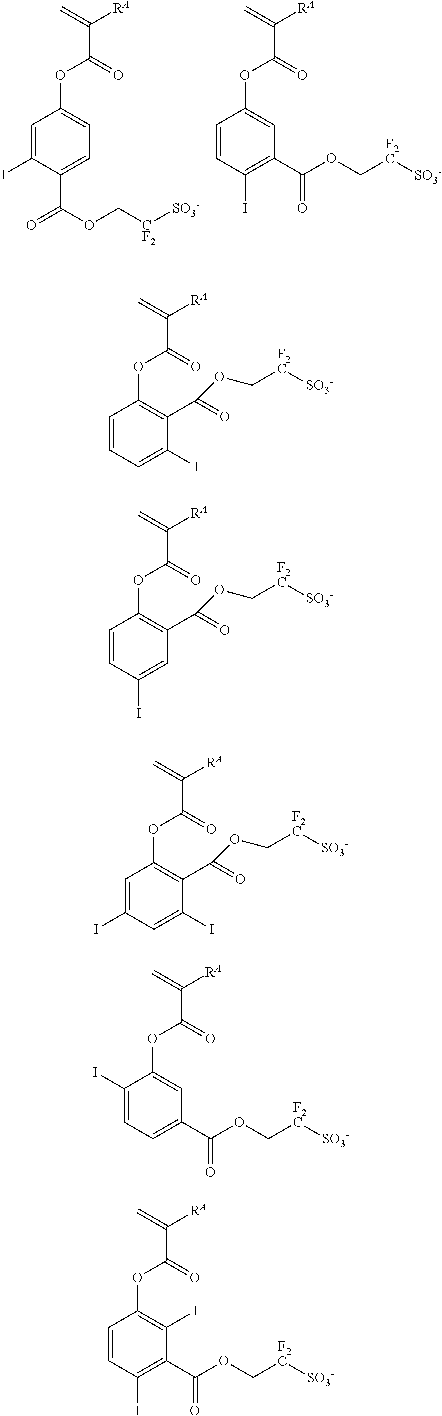

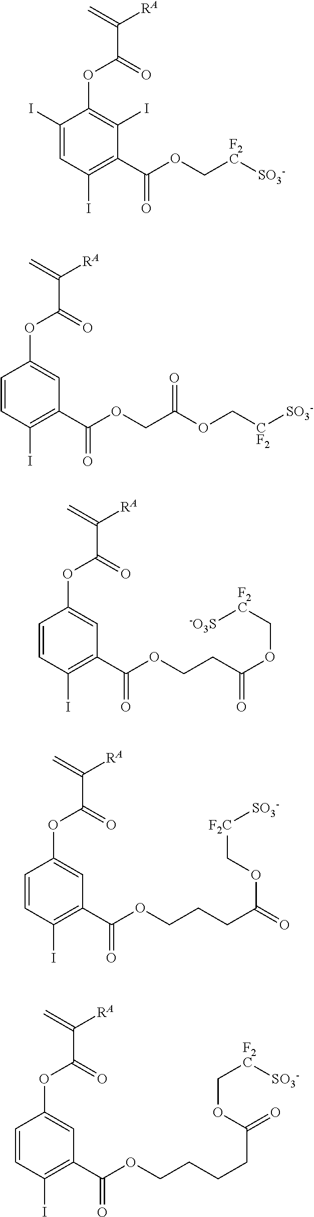

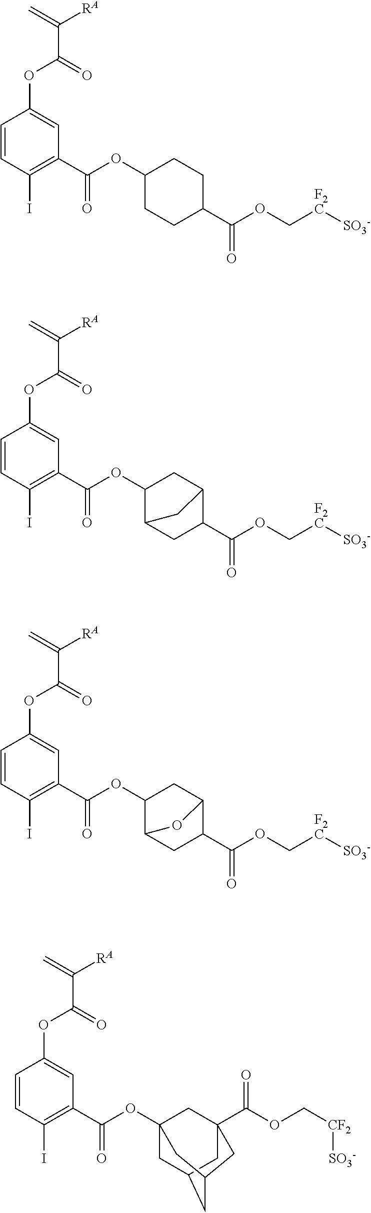

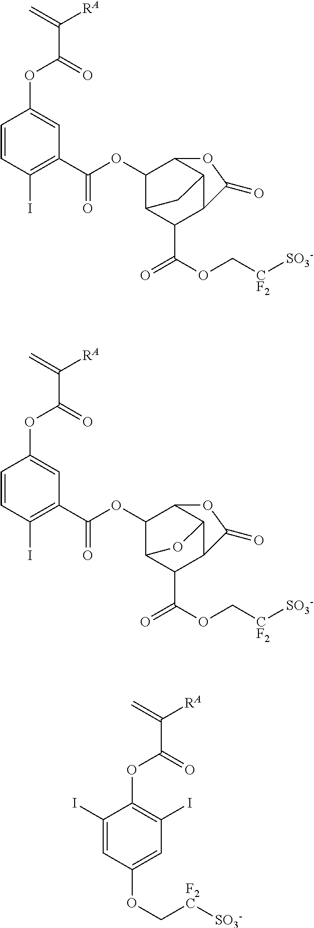

In the formula, R^{A} has the same meanings as in the formula (a1).

Examples of the structural unit (IV) include those having the first onium cation as a counter ion of an anion of the structural unit that provides the first organic acid anion. Examples of the first onium cation include the same ones as the second onium cation of the radiation-sensitive acid generator (C) described below.

When the polymer (A) has the structural unit (IV), the lower limit of the content of the structural unit (IV) (when a plurality of types is contained, the total content thereof is taken) is preferably 0 mol %, more preferably 1 mol %, and still more preferably 5 mol % based on all structural units composing the base polymer (A1). The upper limit of the content is preferably 30 mol %, more preferably 25 mol %, and still more preferably 20 mol %. When the contents of the structural unit (IV) are adjusted to within the above range, a function as an acid generator can be sufficiently exhibited.

### (Structural Unit (V))

The polymer (A) may further contain structural unit (V) containing a polar group such as an alcoholic hydroxy group, a carboxy group, a lactone ring, a sultone ring, an ether group, an ester group, a carbonyl group, or a cyano group. Examples of the monomer that affords the structural unit (V) include, but are not limited to, those shown below. In the following formulas, R^{A} is the same as described above.

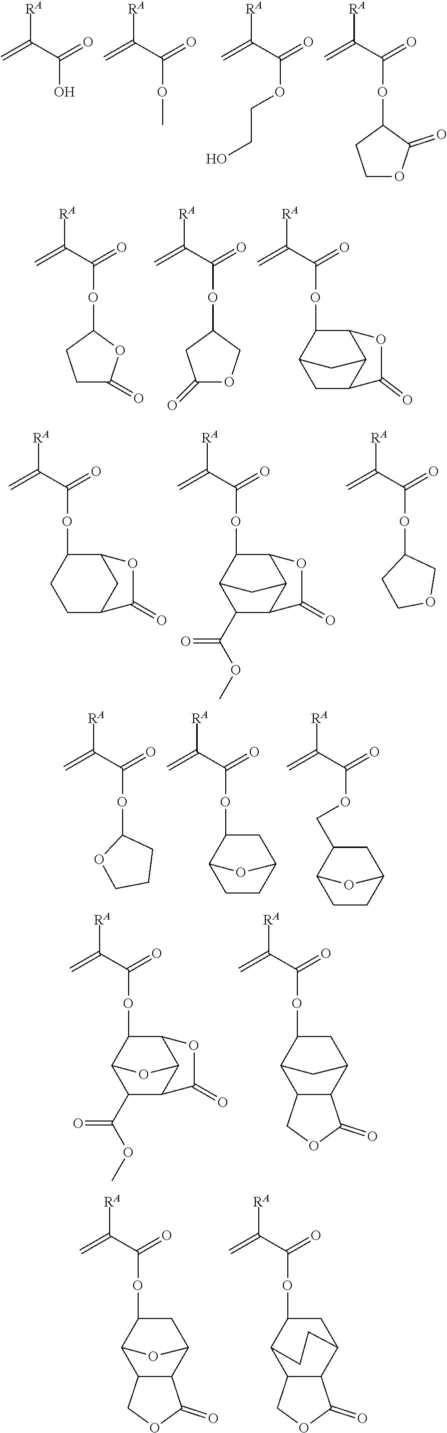

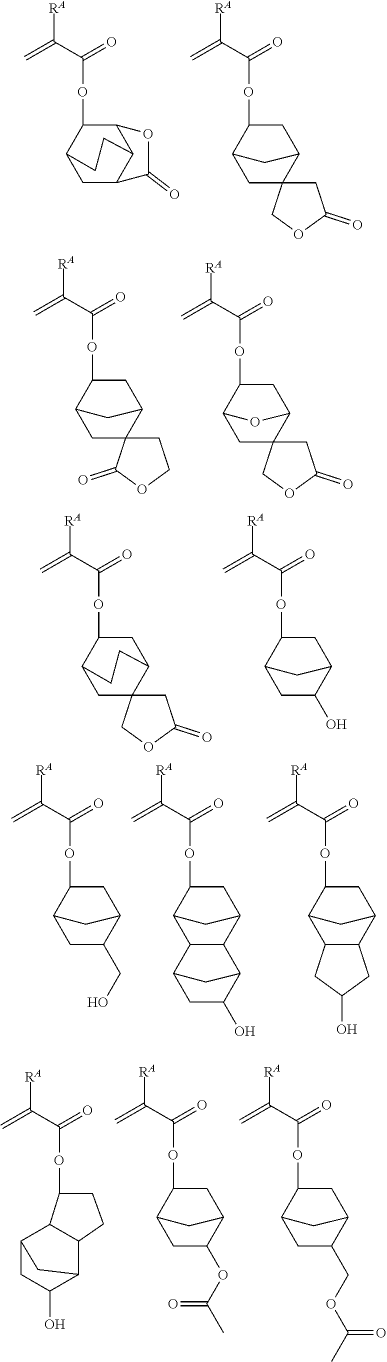

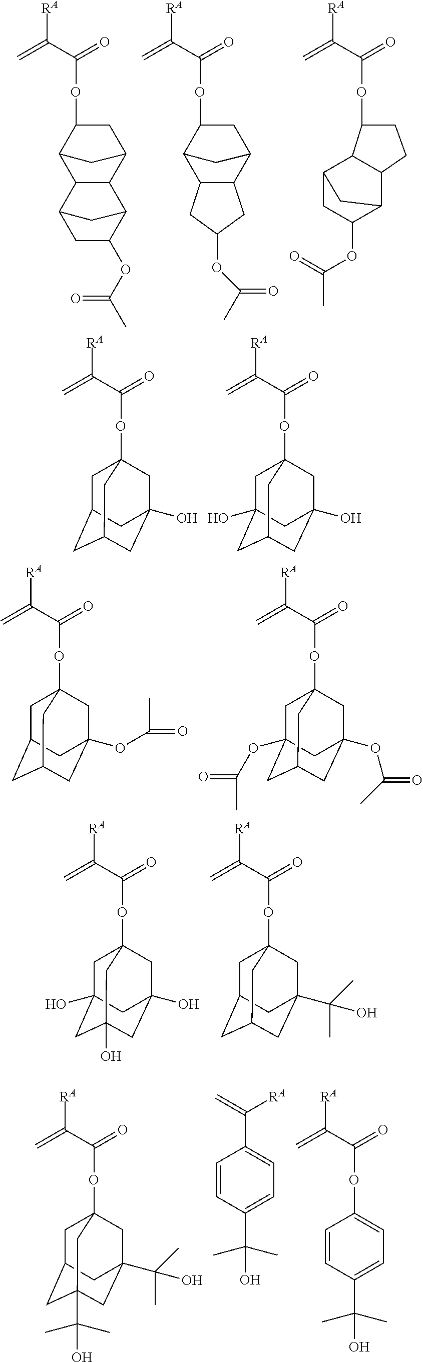

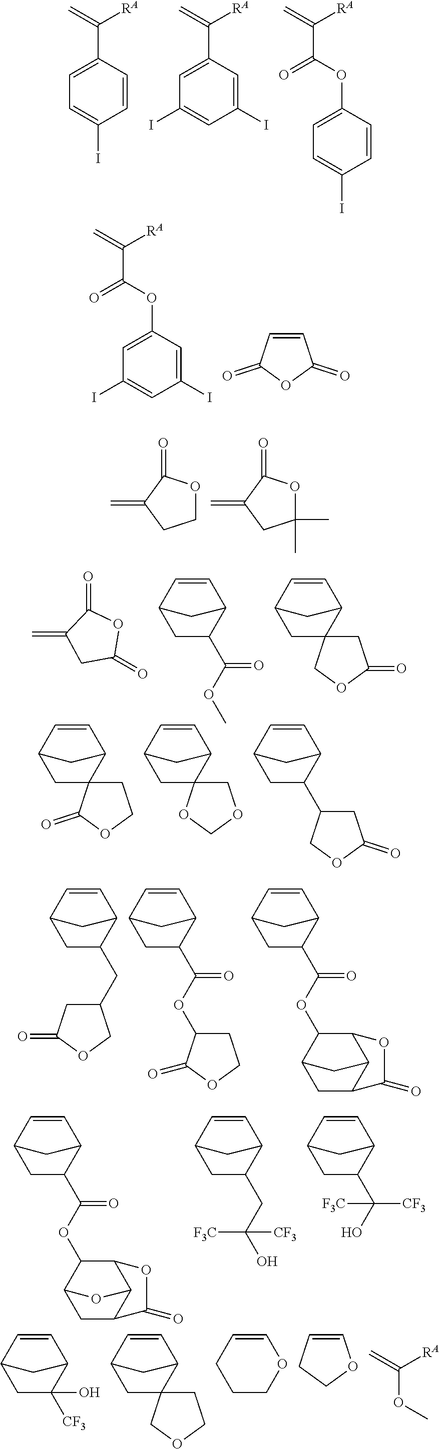

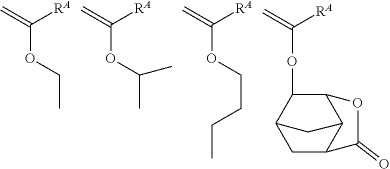

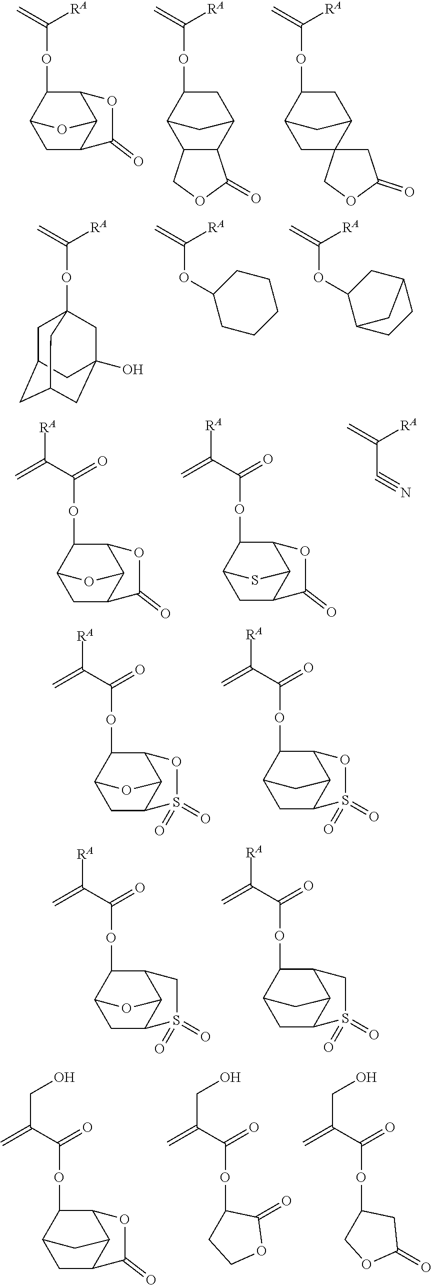

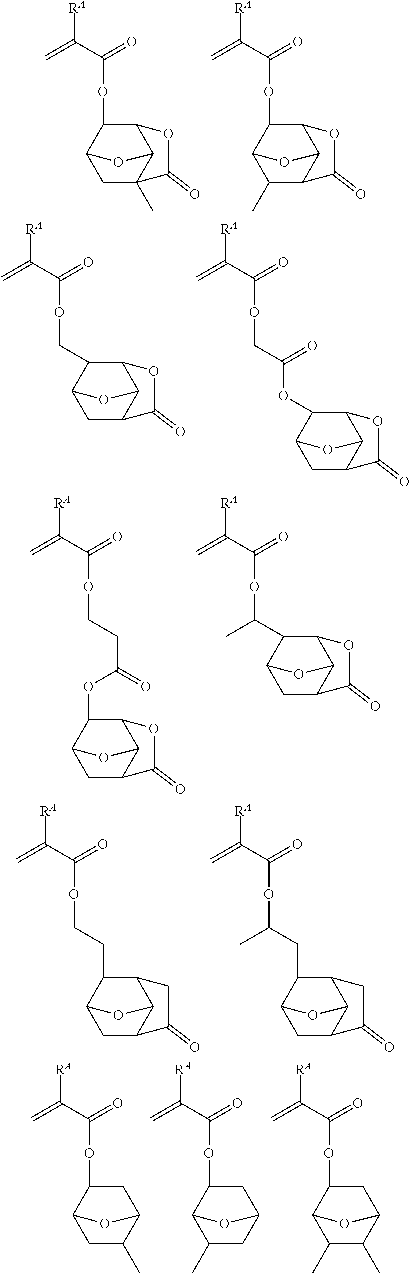

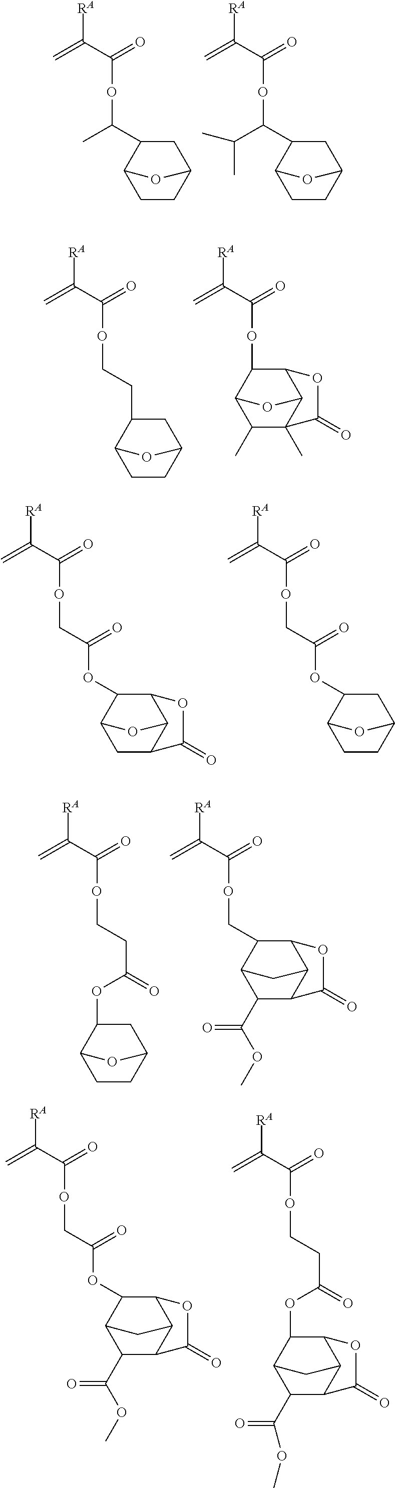

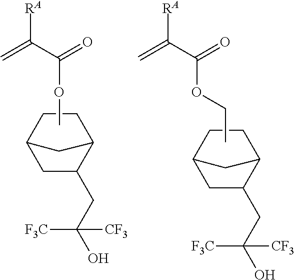

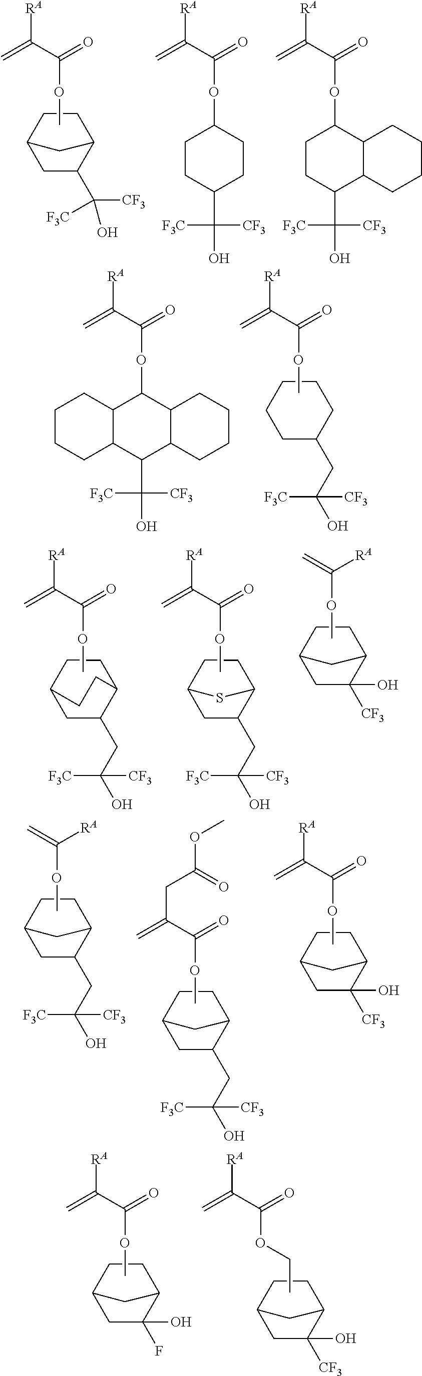

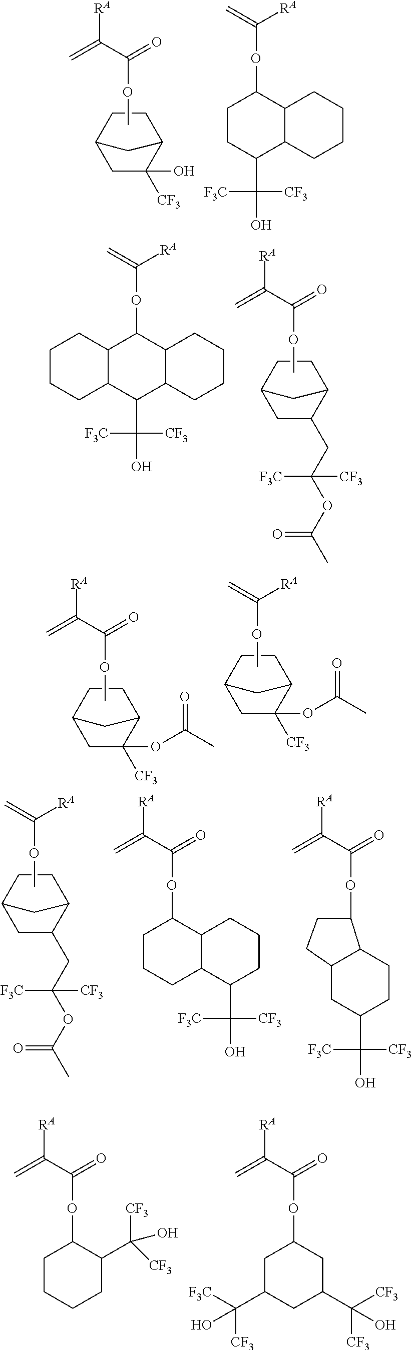

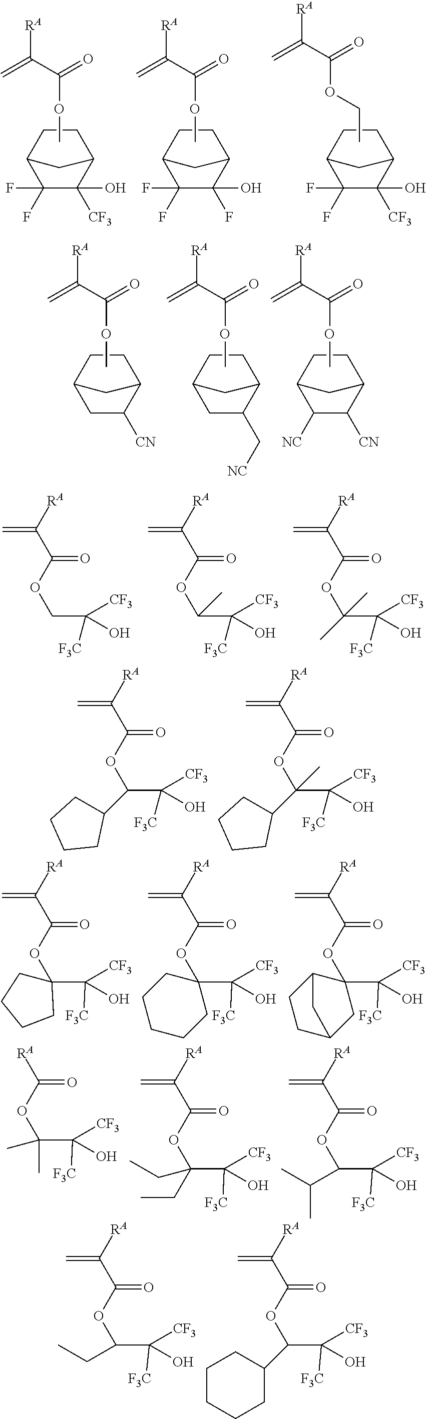

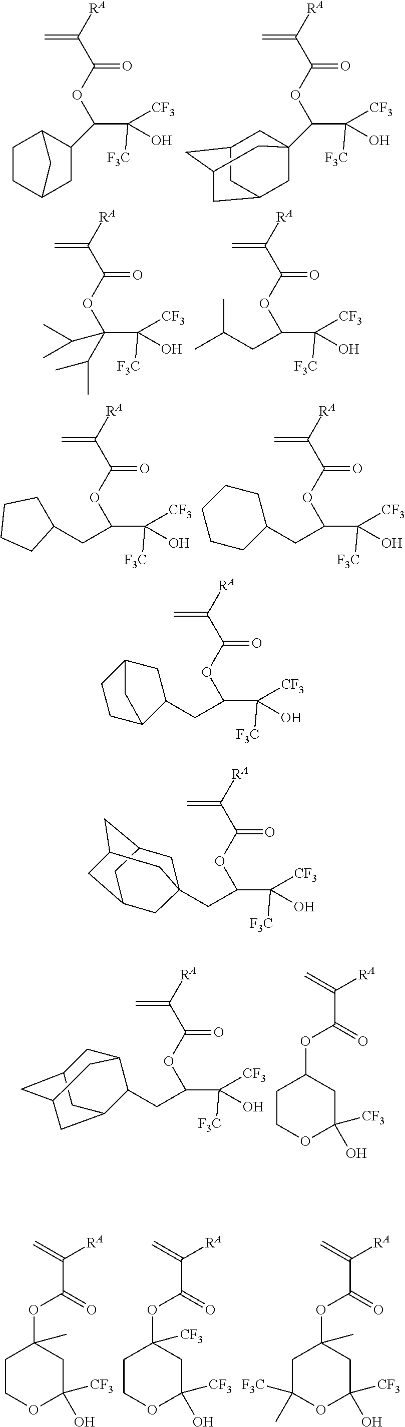

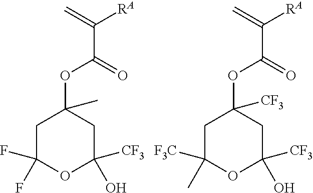

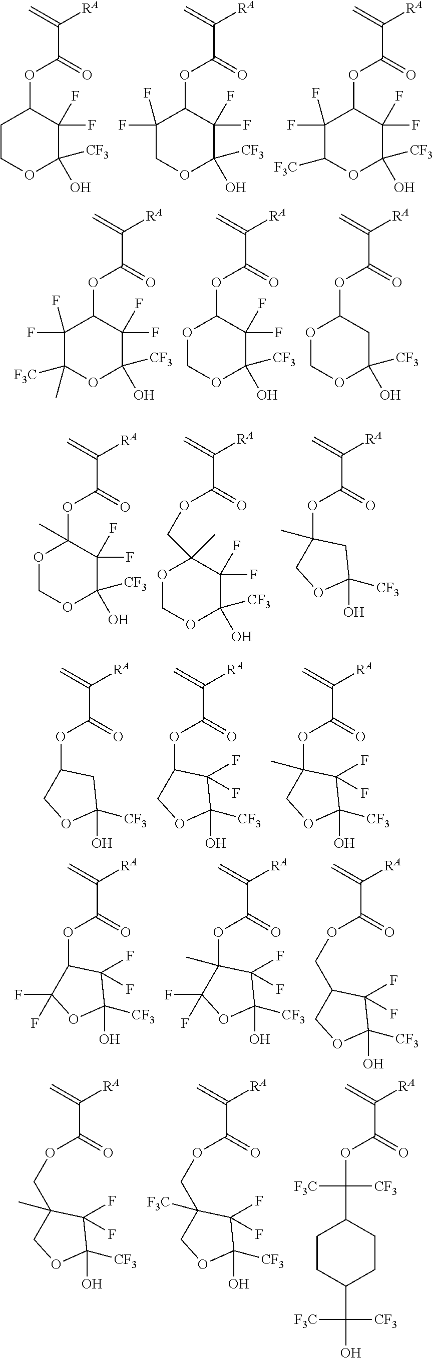

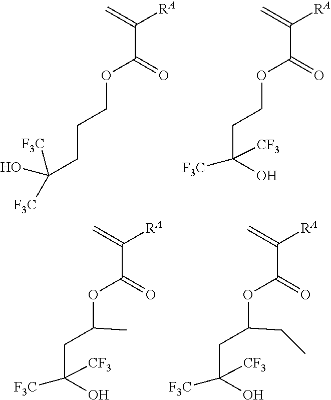

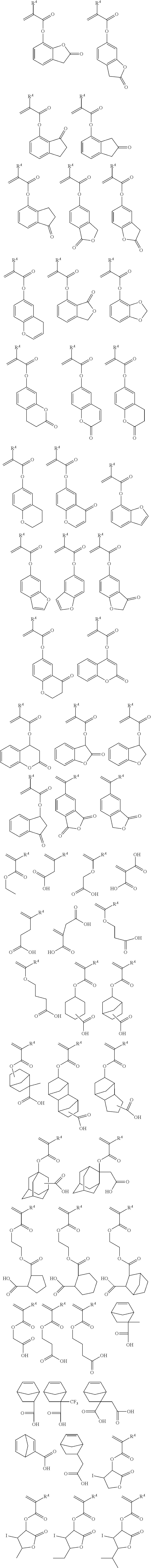

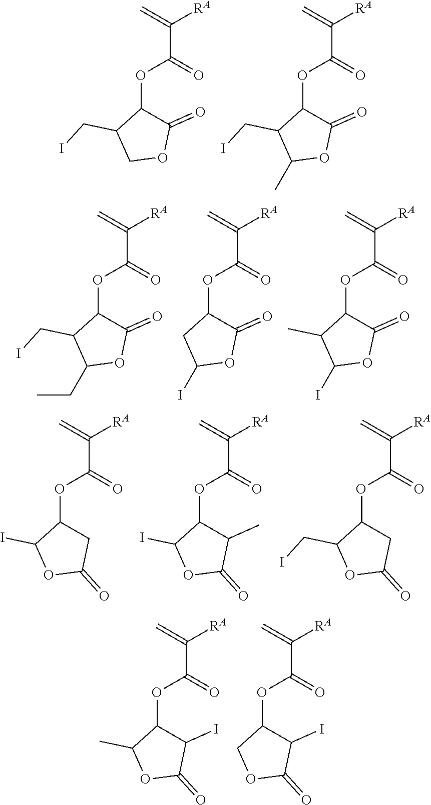

When the base polymer (A) (or the radiation-sensitive acid-generating polymer (A1)) has the structural unit (V), the upper limit of the content of the structural unit (V) (when a plurality of types is contained, the total content thereof is taken) is preferably 40 mol %, more preferably 30 mol % and still more preferably 20 mol % based on all structural units composing the base polymer (or the base polymer (A1)). The lower limit of the content is not particularly limited, and preferably 0 mol %. When the content of the structural unit (V) is adjusted to within the above range, the pattern adhesiveness can be further improved.

### (Method for Synthesizing Polymer (A))

The polymer (A) can be synthesized, for example, by polymerizing monomers to afford the above-described structural units in an organic solvent by heating with addition of a radical polymerization initiator. In the polymerization, a known polymerization initiator can be used.

When hydroxystyrene or hydroxyvinylnaphthalene is copolymerized, acetoxystyrene or acetoxyvinylnaphthalene may be used instead of hydroxystyrene or hydroxyvinylnaphthalene, and an acetoxy group may be deprotected by the alkali hydrolysis after polymerization to form a hydroxystyrene unit or a hydroxyvinylnaphthalene unit. Polymerization may be performed as it is without protecting the hydroxyl group.

The lower limit of the polystyrene-equivalent weight average molecular weight (Mw) of the polymer (A) as measured by gel permeation chromatography (GPC) using THE as a solvent is preferably 2,000, and more preferably 4,000. The upper limit of the Mw is preferably 30,000, and more preferably 15, 000. When the Mw is in the above range, the patternability and heat resistance of a resist material are good.

Furthermore, in a case where the polymer (A) has a wide molecular weight distribution (Mw/Mn), since a low molecular weight or high molecular weight polymer is present, there is a risk that foreign matters may be found on a pattern or the shape of the pattern may be deteriorated after exposure. Since the influence of Mw and molecular weight distribution tends to increase as the pattern rule becomes finer, in order to obtain a resist material suitably used for fine pattern dimensions, the molecular weight distribution of the polymer (A) is preferably a narrow dispersion of 1.0 to 2.0, particularly 1.0 to 1.8.

The polymer (A) may contain two or more polymers differing in composition ratio, Mw, and molecular weight distribution.

The lower limit of the content of the polymer (A) in the radiation-sensitive composition is preferably 50% by mass, more preferably 55% by mass, and still more preferably 60% by mass based on the amount of the radiation-sensitive composition excluding the solvent (B) contained therein. The upper limit of the content is preferably 99% by mass, and more preferably 95% by mass.

### <Radiation-Sensitive Acid Generator (C)>

The radiation-sensitive composition may contain the radiation-sensitive acid generator (C). The radiation-sensitive acid generator (C) contains a second organic acid anion and a second onium cation and is a component that generates an acid through exposure to light. The radiation-sensitive composition of the present invention contains at least one of the radiation-sensitive acid-generating polymer (A1) or the radiation-sensitive acid generator (C), and may contain both.

The radiation-sensitive acid generator (C) is preferably represented by the following formula (p-1).

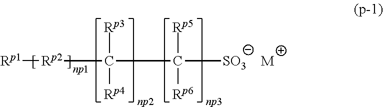

In the formula (p-1), R^{p1} is a monovalent group containing a cyclic structure.
R^{p2} is a divalent linking group.
R^{p3} and R^{p4} are each independently a hydrogen atom, a fluorine atom, a monovalent hydrocarbon group having 1 to 20 carbon atoms, or a monovalent fluorinated hydrocarbon group having 1 to 20 carbon atoms.
R^{p5} and R^{p6} are each independently a fluorine atom or a monovalent fluorinated hydrocarbon group having 1 to 20 carbon atoms.
n^{p1} is an integer of 0 to 10, n^{p2} is an integer of 0 to 10, and n^{p3} is an integer of 0 to 10, provided that n^{p1}+n^{p2}+n^{p3} is an integer of 1 or more and 30 or less.

When n^{p1} is 2 or more, a plurality of R^{p2}s are identical to or different from each other.

When n^{p2} is 2 or more, a plurality of R^{p3}s are identical to or different from each other, and a plurality of R^{p4}s are identical to or different from each other.

When n^{p3} is 2 or more, a plurality of R^{p5}s are identical to or different from each other, and a plurality of R^{p6}s are identical to or different from each other.

M⁺ is a monovalent onium cation.

Examples of the monovalent group containing a cyclic structure represented by R^{p1} include a monovalent group containing an alicyclic structure having 5 or more ring members, a monovalent group containing an aliphatic heterocyclic structure having 5 or more ring members, a monovalent group containing an aromatic ring structure having 6 or more ring members, and a monovalent group containing an aromatic heterocyclic structure having 6 or more ring members.

Examples of the alicyclic structure having 5 or more ring members include monocyclic cycloalkane structures such as a cyclopentane structure, a cyclohexane structure, a cycloheptane structure, a cyclooctane structure, a cyclononane structure, a cyclodecane structure, and a cyclododecane structure; monocyclic cycloalkene structures such as a cyclopentene structure, a cyclohexene structure, a cycloheptene structure, a cyclooctene structure, and a cyclodecene structure; polycyclic cycloalkane structures such as a norbornane structure, an adamantane structure, a tricyclodecane structure, and a tetracyclododecane structure; and polycyclic cycloalkene structures such as a norbornene structure and a tricyclodecene structure.

Examples of the aliphatic heterocyclic structure having 5 or more ring members include lactone structures such as a pentanolactone structure, a hexanolactone structure, and a norbornane lactone structure; a sultone structure such as a pentanosultone structure, a hexanosultone structure, and a norbornane sultone structure; an oxygen-containing heterocyclic structure such as an oxacyclopentane structure, an oxacycloheptane structure, and an oxanorbornane structure; a nitrogen-containing heterocyclic structures such as an azacyclopentane structure, an azacyclohexane structure, and a diazabicyclooctane structure; and a sulfur-containing heterocyclic structures such as a thiacyclopentane structure, a thiacyclohexane structure, and a thianorbornane structure.

Examples of the aromatic ring structure having 6 or more ring members include a benzene structure, a naphthalene structure, a phenanthrene structure, and an anthracene structure.

Examples of the aromatic heterocyclic structure having 6 or more ring members include oxygen-containing heterocyclic structures such as a furan structure, a pyran structure, and a benzopyran structure; and nitrogen-containing heterocyclic structures such as a pyridine structure, a pyrimidine structure, and an indole structure.

The lower limit of the number of ring members in the cyclic structure of R^{p1} is not particularly limited, but is preferably 5, and more preferably 6. The upper limit of the number of ring members is also not particularly limited, but is preferably 15, and more preferably 12. By setting the number of ring members within the above range, the diffusion length of the acid described above can be further appropriately shortened, which is preferable.

Among them, the R^{p1} is preferably a monovalent group containing an alicyclic structure having 5 or more ring members or a monovalent group containing an aromatic heterocyclic structure having 6 or more ring members, specifically more preferably an adamantyl group, a phenyl group, or a naphthyl group, particularly preferably a phenyl group.

Some or all of the hydrogen atoms of the cyclic structure of R^{p1} may be replaced by a substituent. As the substituent, those exemplified as the substituent that can be possessed by the divalent linking group represented by L^{A1} of the formula (1) can be suitably employed. Among them, an iodine atom, a hydroxy group, an alkoxy group, and a carboxy group are preferable.

As the divalent linking group represented by R^{p2}, a divalent linking group represented by L¹ in the formula (1) can be suitably employed.

As the monovalent hydrocarbon group having 1 to 20 carbon atoms represented by R^{p3} and R^{p4}, a monovalent hydrocarbon group having 1 to 20 carbon atoms represented by R³ of the formula (1) can be suitably employed. Examples of the monovalent fluorinated hydrocarbon group having 1 to 20 carbon atoms represented by R^{p3}, R^{p4}, R^{p5}, and R^{p6} include groups in which some or all of hydrogen atoms of the monovalent hydrocarbon group having 1 to 20 carbon atoms are replaced by fluorine atoms.
R^{p3} and R^{p4} are each independently preferably a hydrogen atom or an alkyl group having 1 to 5 carbon atoms, and more preferably a hydrogen atom. R^{p5} and R^{p6} are preferably a fluorine atom or a fluorinated alkyl group, more preferably a fluorine atom or a perfluoroalkyl group, still more preferably a fluorine atom or a trifluoromethyl group, and particularly preferably a fluorine atom.
n^{p1} is preferably an integer of 0 to 5, more preferably an integer of 0 to 3, still more preferably an integer of 0 to 2, and particularly preferably 0 or 1.
n^{p2} is more preferably an integer of 0 to 5.
n^{p3} is preferably an integer of 0 to 5, more preferably an integer of 1 to 4, still more preferably an integer of 1 to 3, and particularly preferably 1 or 2. By setting n^{p3} to 1 or more, the strength of the acid generated from the compound of formula (p-1) can be increased, and as a result, CDU and the like of the radiation-sensitive composition can be further improved. The upper limit of n^{p3} is preferably 4, more preferably 3, and still more preferably 2.

In formula (p-1), n^{p1}+n^{p2}+n^{p3} is an integer of 1 to 30. The lower limit of n^{p1}+n^{p2}+n^{p3} is preferably 2, more preferably 3. The upper limit of n^{p1}+n^{p2}+n^{p3} is preferably 10, more preferably 5.

Examples of the second organic acid anion of the radiation-sensitive acid generator (C) represented by the formula (p-1) include, but are not limited to, those shown below. Among those shown below, as the organic acid anion having an iodine group-containing aromatic ring structure, a structure in which an iodine atom is replaced by an atom or a group other than an iodine atom such as a hydrogen atom or another substituent can also be suitably employed.

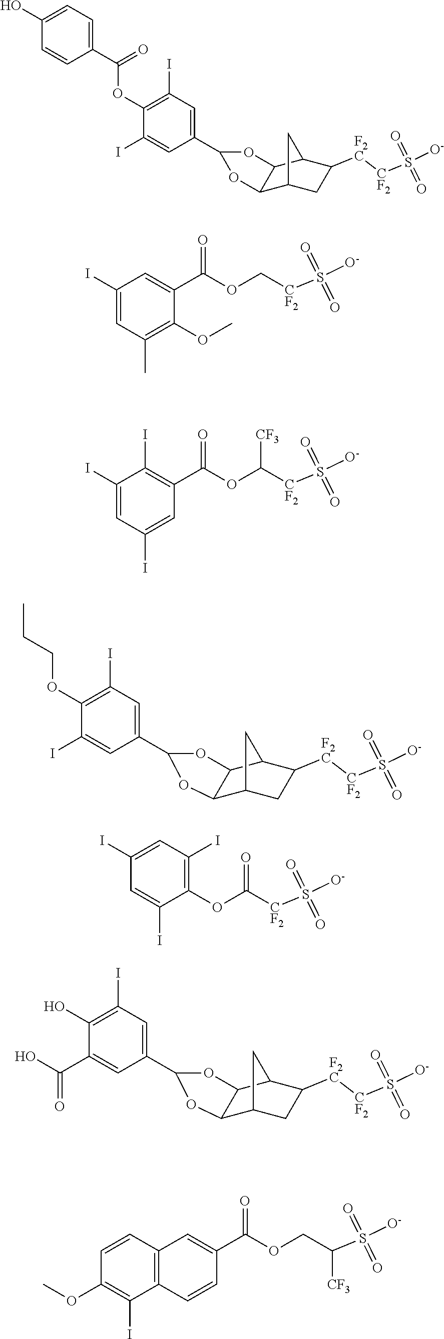

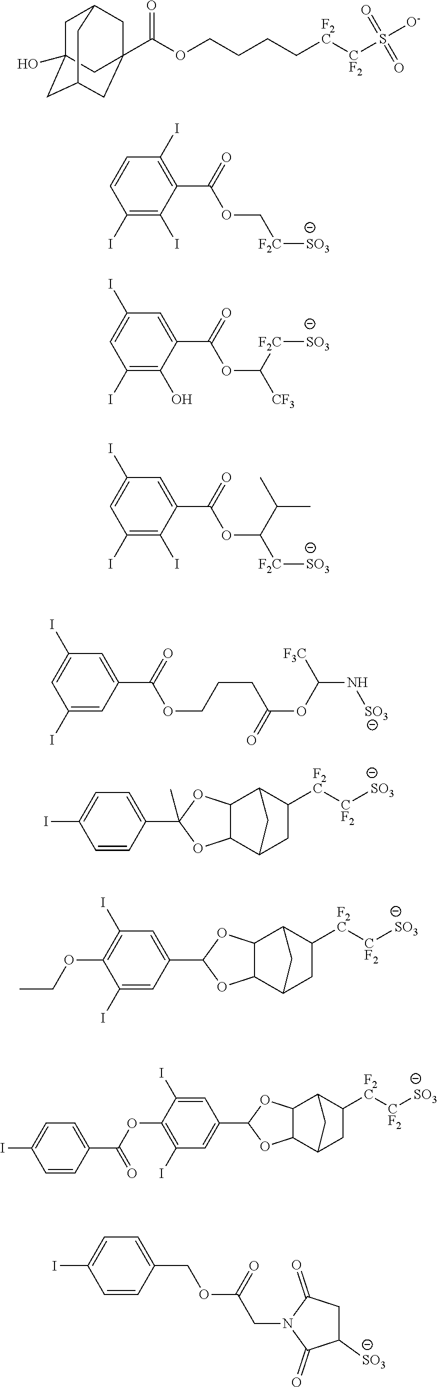

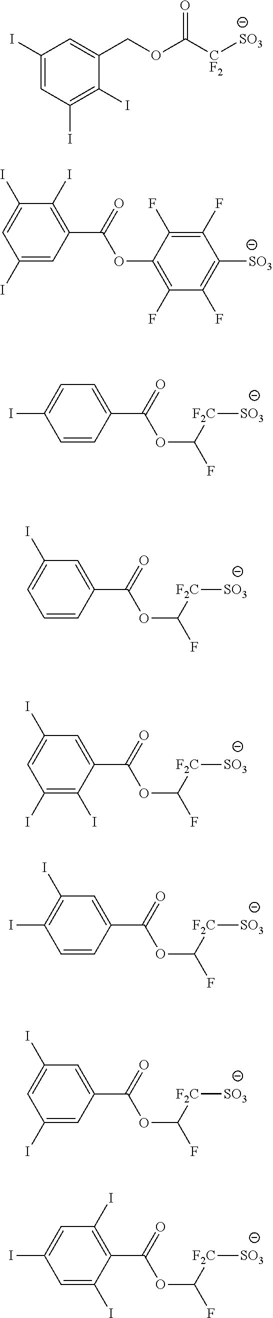

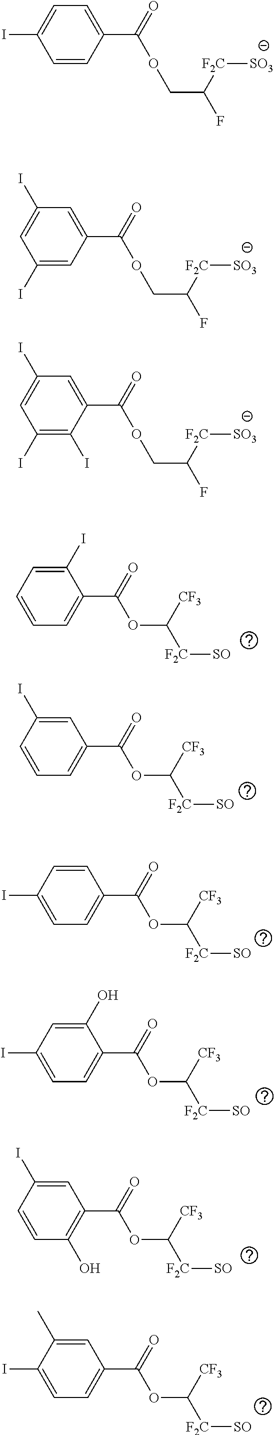

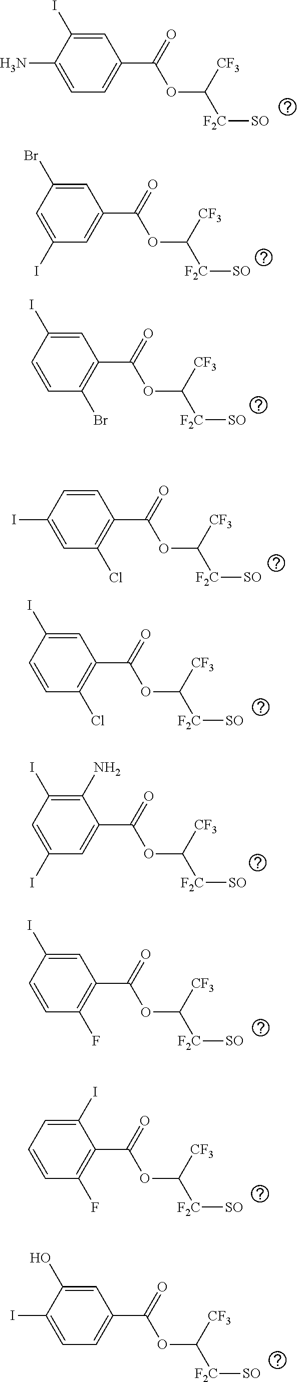

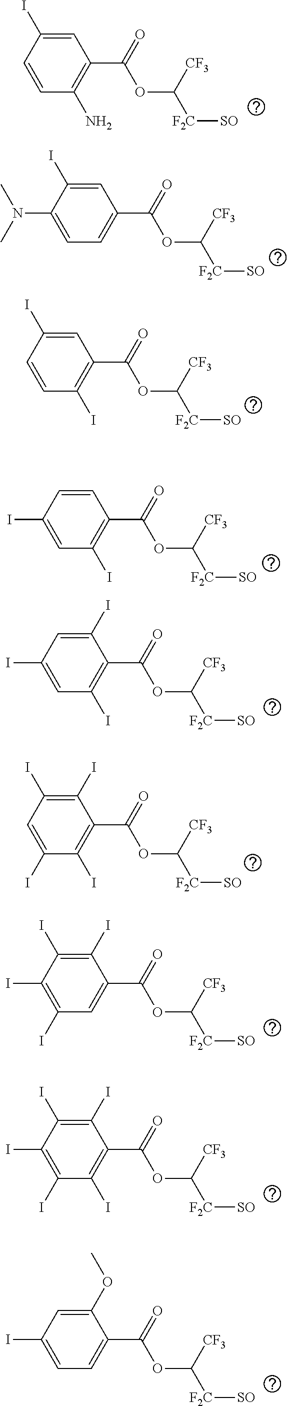

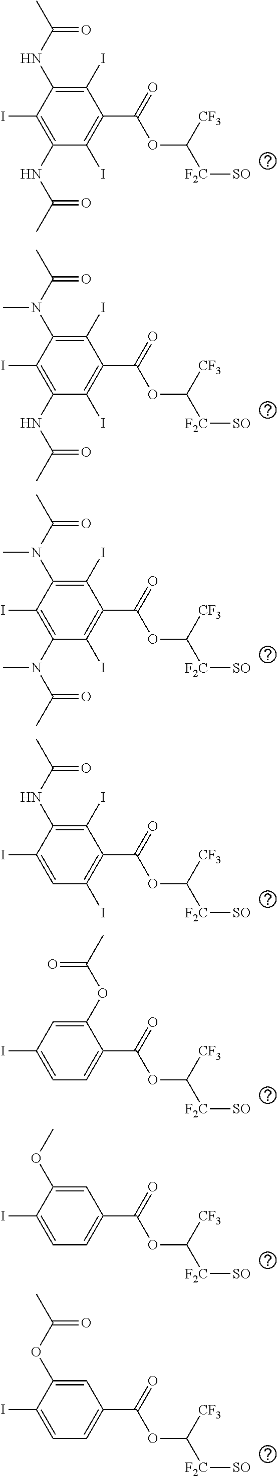

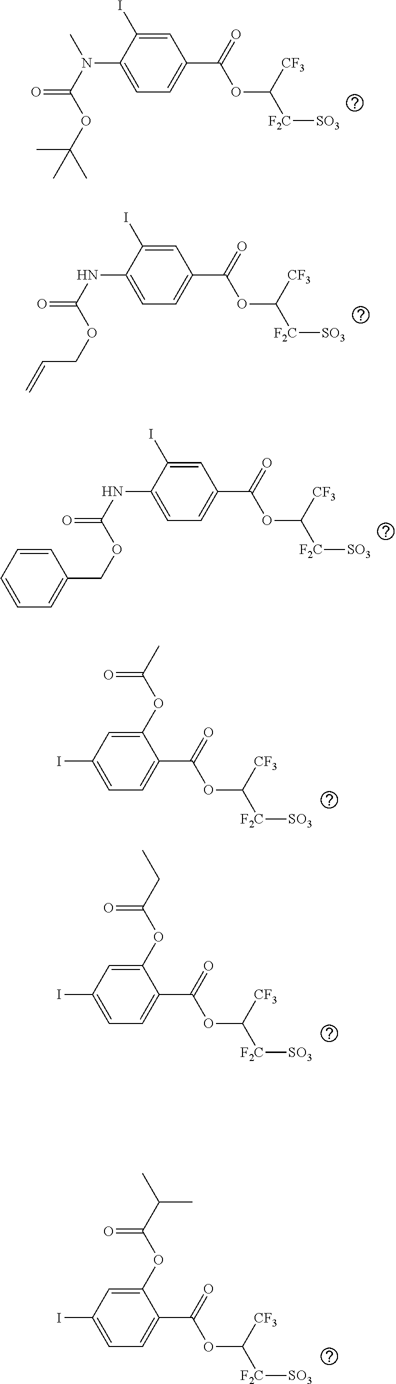

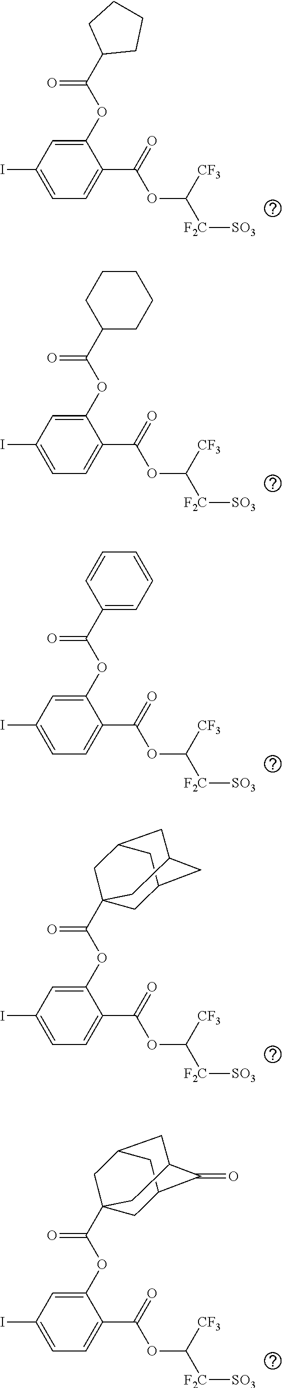

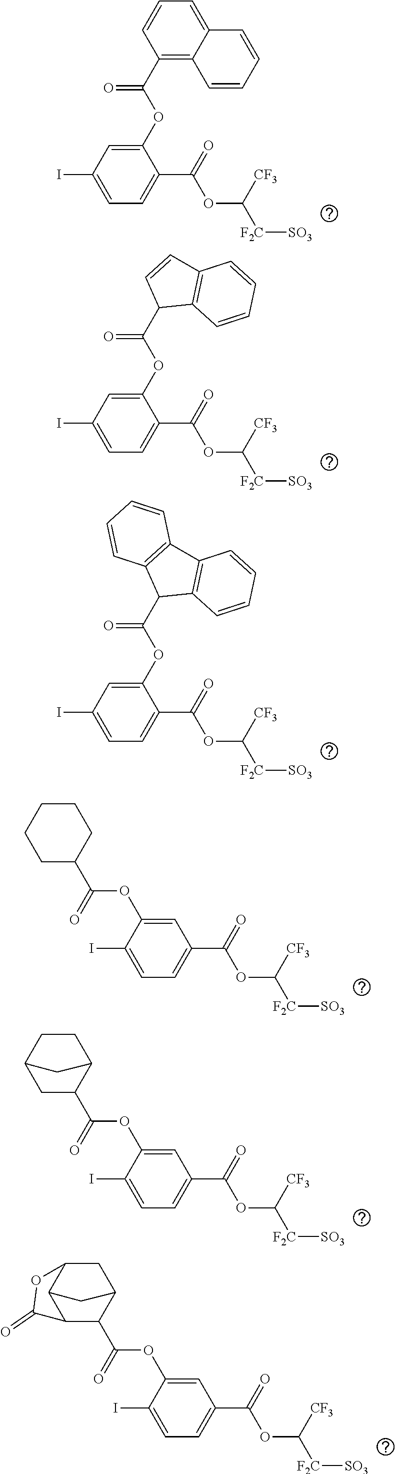

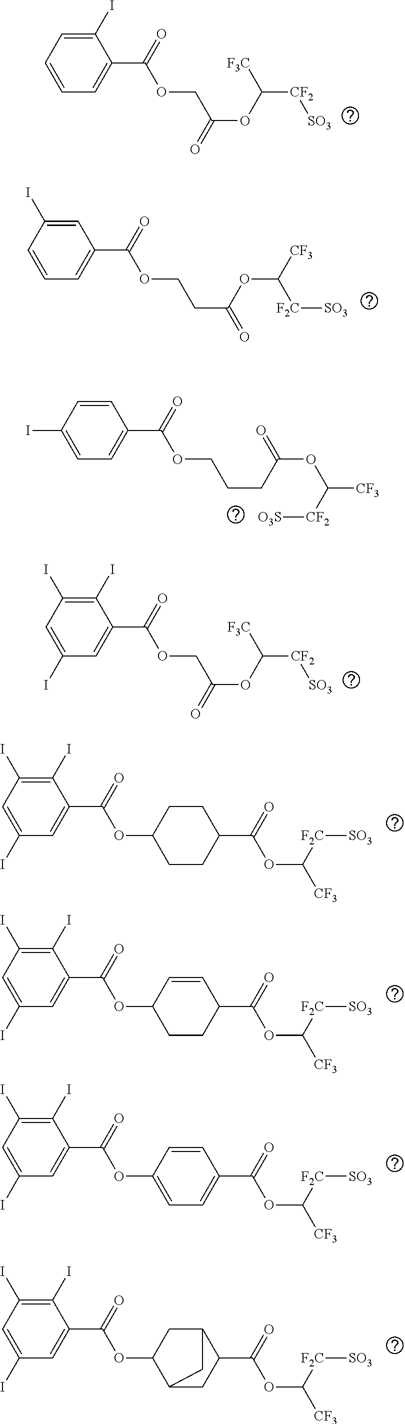

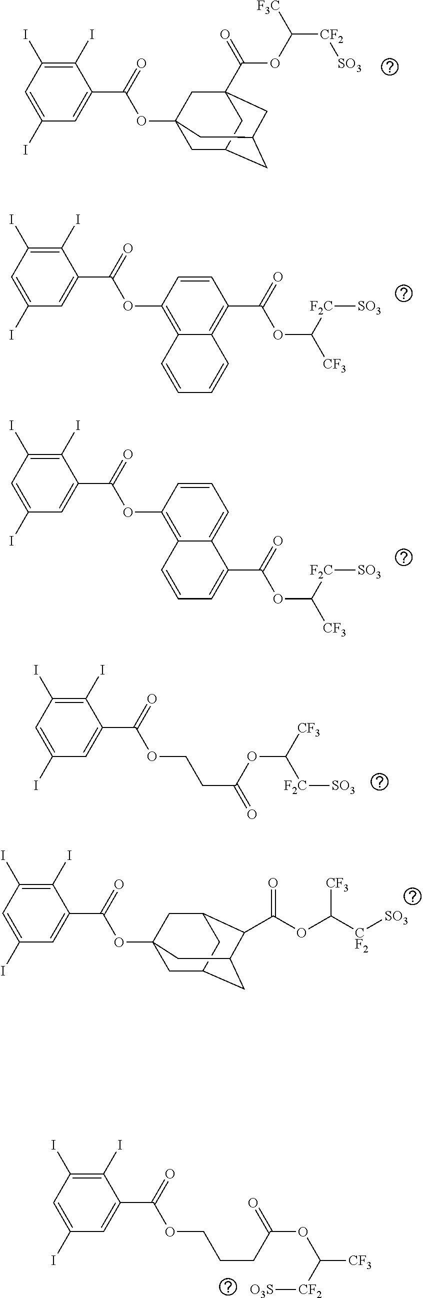

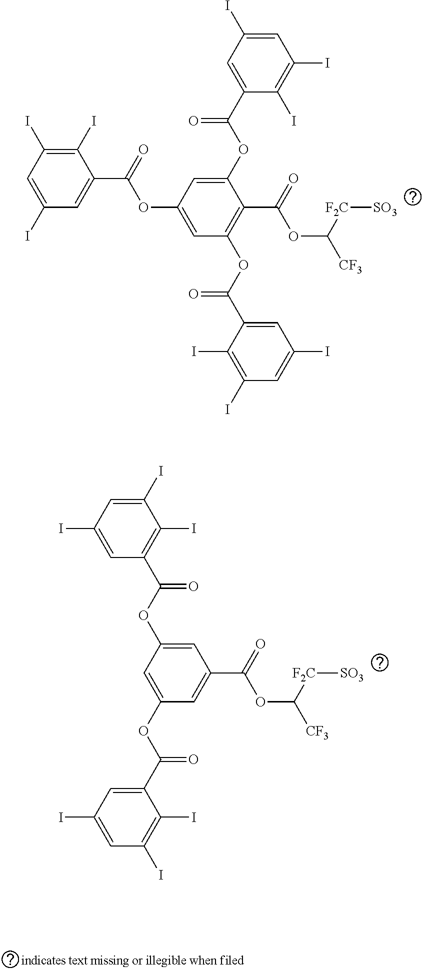

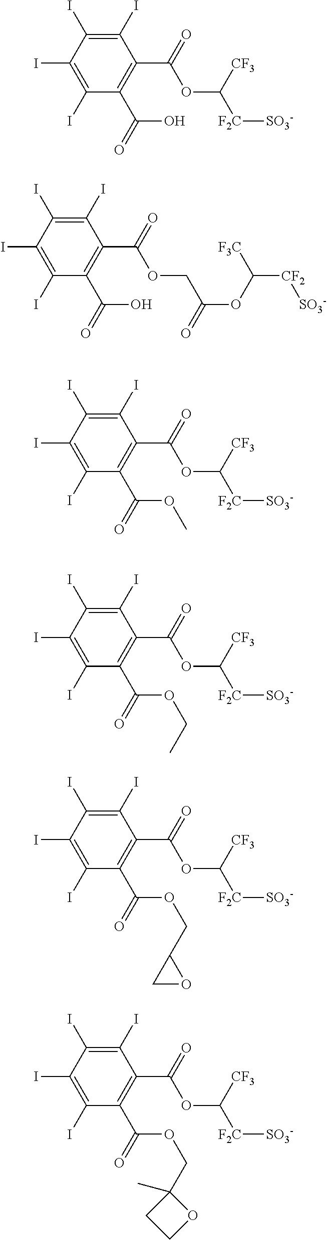

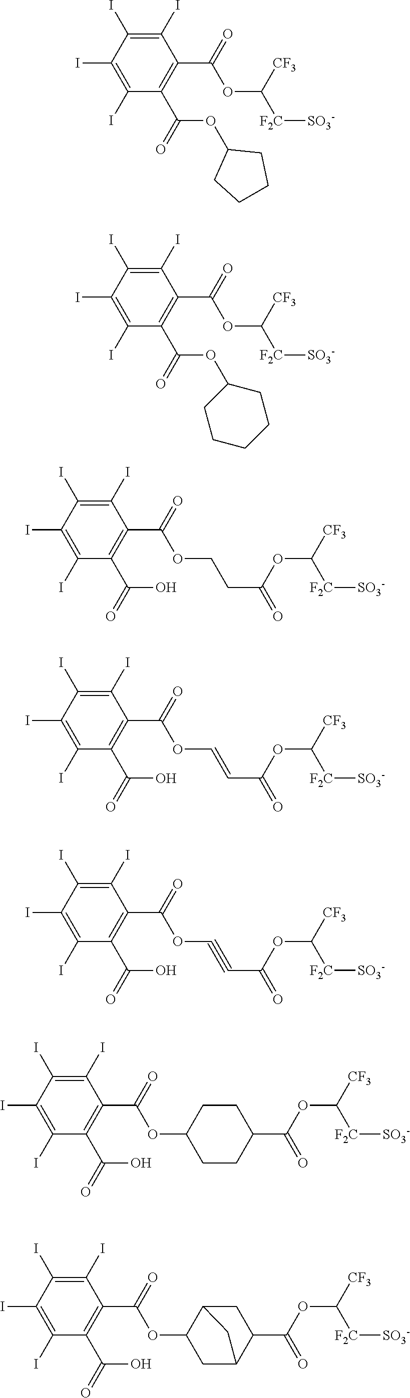

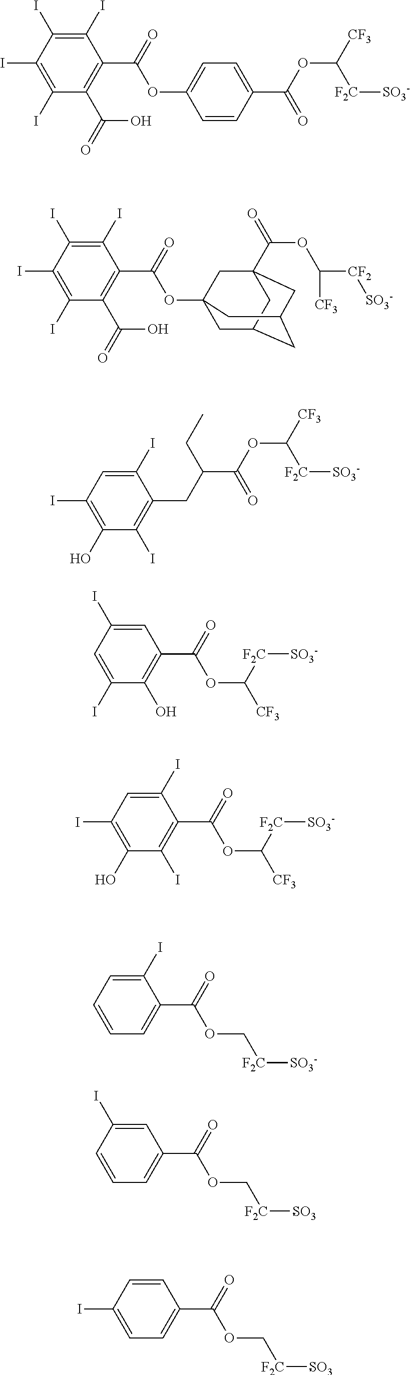

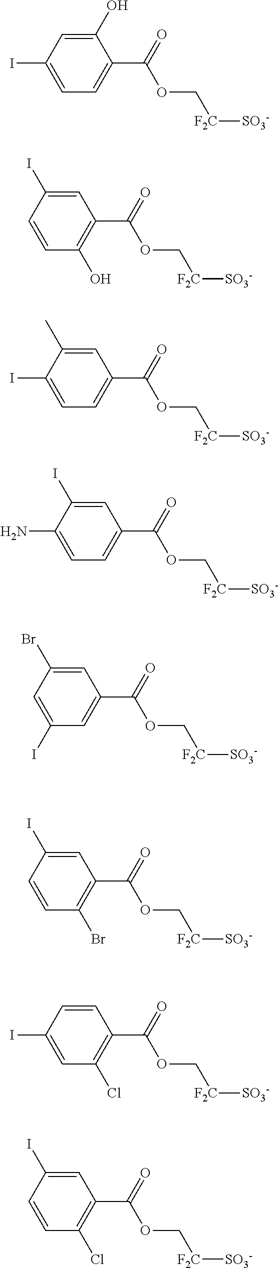

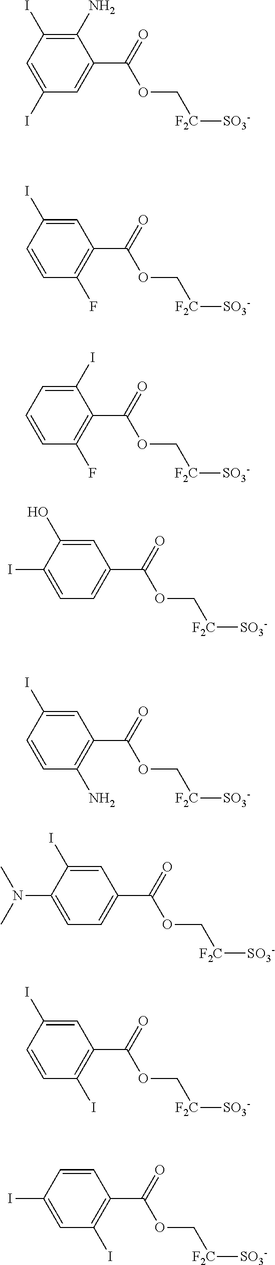

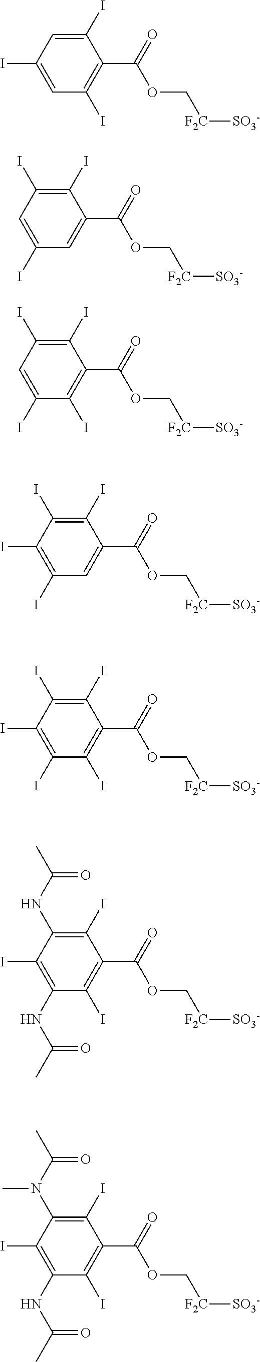

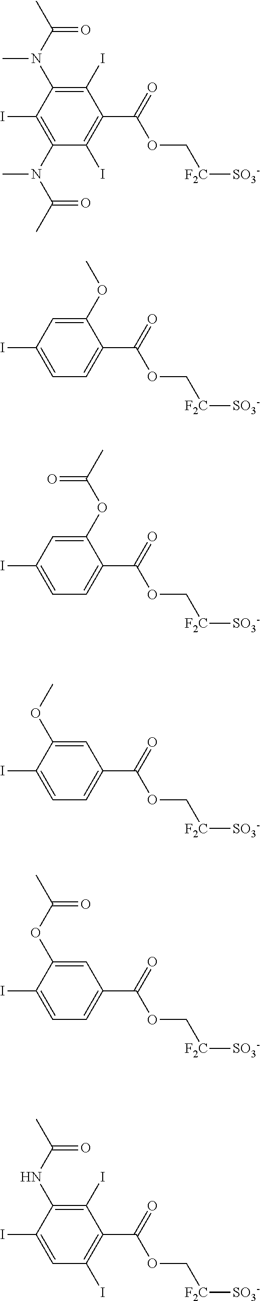

An example of the second onium cation represented by M⁺ of the formula (p-1) is a radioactive ray-degradable onium cation containing an element such as S, I, O, N, P, Cl, Br, F, As, Se, Sn, Sb, Te, or Bi. Examples of such a radioactive ray-degradable onium cation include a sulfonium cation, a tetrahydrothiophenium cation, a iodonium cation, a phosphonium cation, a diazonium cation, and a pyridinium cation. Among them, a sulfonium cation or a iodonium cation is preferred. The sulfonium cation or the iodonium cation is preferably represented by any of the formulas (X-1) to (X-6).

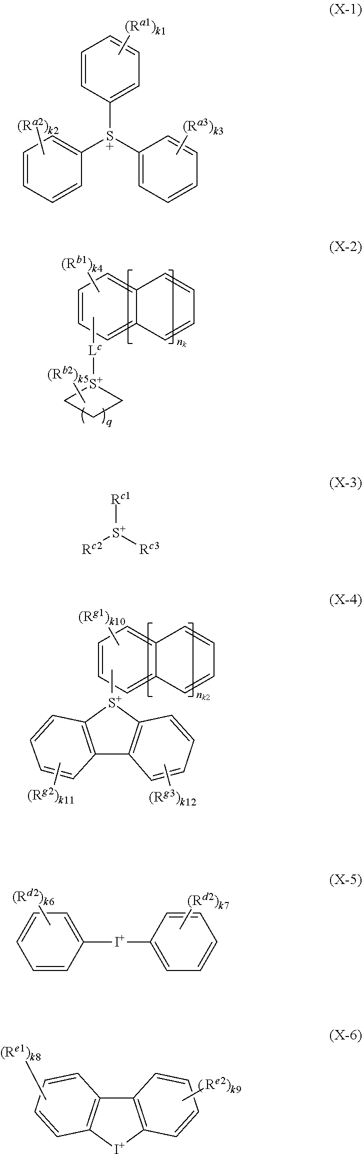

In the formula (X-1), R^{a1}, R^{a2} and R^{a3} are each independently a substituted or unsubstituted, linear or branched chain alkyl group, alkoxy group, or alkoxycarbonyloxy group having 1 to 12 carbon atoms; a substituted or unsubstituted, monocyclic or polycyclic cycloalkyl group having 3 to 12 carbon atoms; a substituted or unsubstituted aromatic hydrocarbon group having 6 to 12 carbon atoms; a hydroxy group, a halogen atom, —OSO₂—R^{P}, —SO₂—R^{P}, —S—R^{T}, —O—, —CO— or a combination thereof; or a ring structure obtained by combining two or more of these groups. The ring structure may contain heteroatoms such as O and S between the carbon-carbon bonds forming the skeleton. R^{P}, R^{Q} and R^{T} are each independently a substituted or unsubstituted, linear or branched chain alkyl group having 1 to 12 carbon atoms; a substituted or unsubstituted alicyclic hydrocarbon group having 5 to 25 carbon atoms; or a substituted or unsubstituted aromatic hydrocarbon group having 6 to 12 carbon atoms. k1, k2 and k3 are each independently an integer of 0 to 5. When there are a plurality of R^{a1} to R^{a3} and a plurality of R^{P}, R^{Q} and R^{T}, a plurality of R^{a1} to R^{a3} and a plurality of R^{P}, R^{Q} and R^{T} may be each identical or different.

In the formula (X-2), R^{b1} is a substituted or unsubstituted, linear or branched chain alkyl group, alkoxy group or alkoxyalkyloxy group having 1 to 20 carbon atoms; a substituted or unsubstituted acyl group having 2 to 8 carbon atoms; or a substituted or unsubstituted aromatic hydrocarbon group having 6 to 8 carbon atoms; or a hydroxy group. nₖ is 0 or 1. When nₖ is 0, k4 is an integer of 0 to 4. When nₖ is 1, k4 is an integer of 0 to 7. When there are a plurality of R^{b1}, a plurality of R^{b1} may be each identical or different. A plurality of R^{b1} may represent a ring structure obtained by combining them. R^{b2} is a substituted or unsubstituted, linear or branched chain alkyl group having 1 to 7 carbon atoms; or a substituted or unsubstituted aromatic hydrocarbon group having 6 or 7 carbon atoms. Le is a single bond or divalent linking group. k5 is an integer of 0 to 4. When there are a plurality Of R^{b2}, a plurality of R^{b2} may be each identical or different. A plurality of R^{b2} may represent a ring structure obtained by combining them. q is an integer of 0 to 3. In the formula, the ring structure containing S⁺ may contain a heteroatom such as O or S between the carbon-carbon bonds forming the skeleton.

In the formula (X-3), R^{c1}, R^{c2} and R^{c3} are each independently a substituted or unsubstituted, linear or branched chain alkyl group having 1 to 12 carbon atoms.

In the formula (X-4), R^{g1} is a substituted or unsubstituted linear or branched chain alkyl or alkoxy group having 1 to 20 carbon atoms, a substituted or unsubstituted acyl group having 2 to 8 carbon atoms, a substituted or unsubstituted aromatic hydrocarbon group having 6 to 8 carbon atoms, or a hydroxy group. nₖ2 is 0 or 1. When nₖ2 is 0, k10 is an integer of 0 to 4, and when nₖ2 is 1, k10 is an integer of 0 to 7. When there are two or more R^{g1}s, the two or more R^{g1}s are the same or different from each other, and may represent a cyclic structure formed by combining them together. R^{g2} and R^{g3} are each independently a substituted or unsubstituted linear or branched chain alkyl, alkoxy, or alkoxycarbonyloxy group having 1 to 12 carbon atoms, a substituted or unsubstituted monocyclic or polycyclic cycloalkyl group having 3 to 12 carbon atoms, a substituted or unsubstituted aromatic hydrocarbon group having 6 to 12 carbon atoms, a hydroxyl group, a halogen atom, or a ring structure formed by combining two or more of these groups together. k11 and k12 are each independently an integer of 0 to 4. When there are two or more R^{g2}s and two or more R^{g3}s, the two or more R^{g2}s may be the same or different from each other, and the two or more R^{g3}s may be the same or different from each other.

In the formula (X-5), R^{d1} and R^{d2} are each independently a substituted or unsubstituted, linear or branched chain alkyl group, alkoxy group or alkoxycarbonyl group having 1 to 12 carbon atoms; a substituted or unsubstituted aromatic hydrocarbon group having 6 to 12 carbon atoms; a halogen atom; a halogenated alkyl group having 1 to 4 carbon atoms; a nitro group; or a ring structure obtained by combining two or more of these groups. k6 and k7 are each independently an integer of 0 to 5. When there are a plurality of R^{d1} and a plurality of R^{d2}, a plurality of R^{d1} and a plurality of R^{d2} may be each identical or different.

In the formula (X-6), R^{e1} and R^{e2} are each independently a halogen atom; a substituted or unsubstituted linear or branched chain alkyl group having 1 to 12 carbon atoms; or a substituted or unsubstituted aromatic hydrocarbon group having 6 to 12 carbon atoms. k8 and k9 are each independently an integer of 0 to 4.

Specific examples of the second onium cation include, but not limited thereto, the following structures.

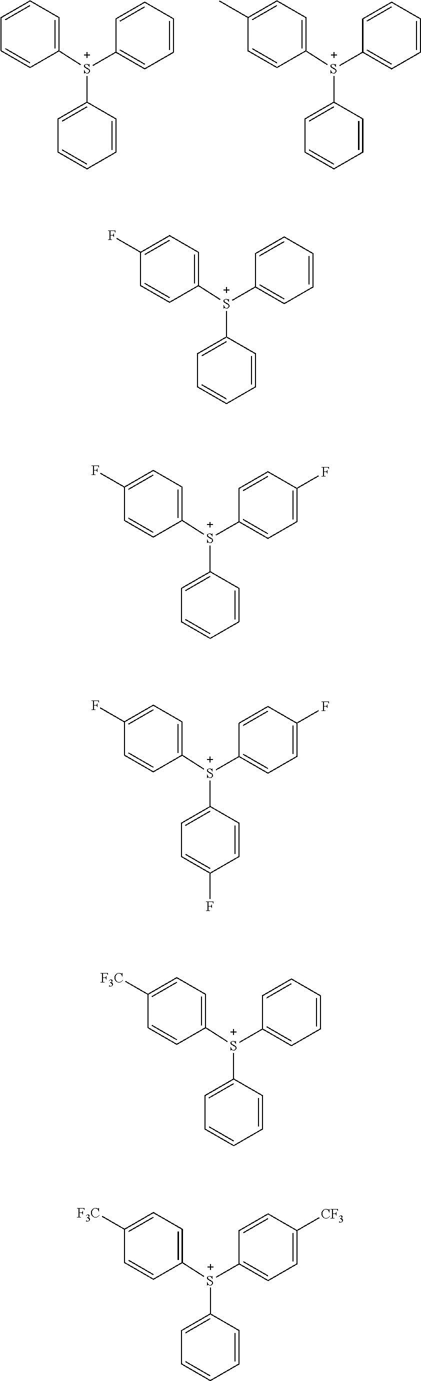

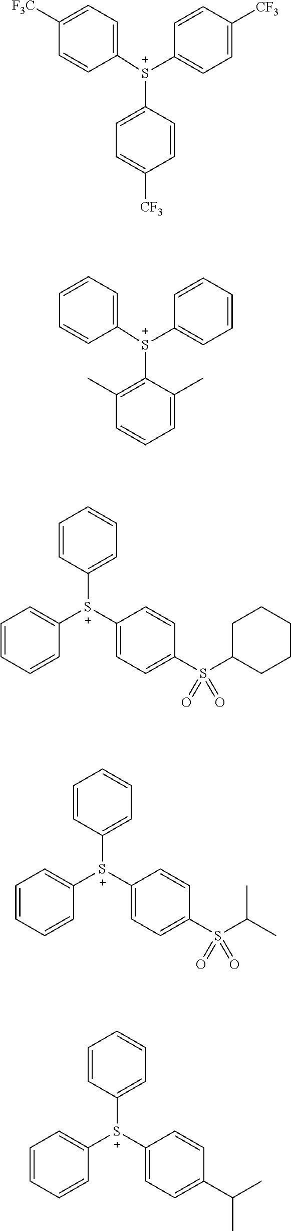 [SMILES: FC(F)(F)c1ccc([S+](c2ccc(C(F)(F)F)cc2)c2ccc(C(F)(F)F)cc2)cc1 ; Cc1cccc(C)c1[S+](c1ccccc1)c1ccccc1 ; O=S(=O)(c1ccc([S+](c2ccccc2)c2ccccc2)cc1)C1CCCCC1 ; CC(C)S(=O)(=O)c1ccc([S+](c2ccccc2)c2ccccc2)cc1 ; CC(C)c1ccc([S+](c2ccccc2)c2ccccc2)cc1]

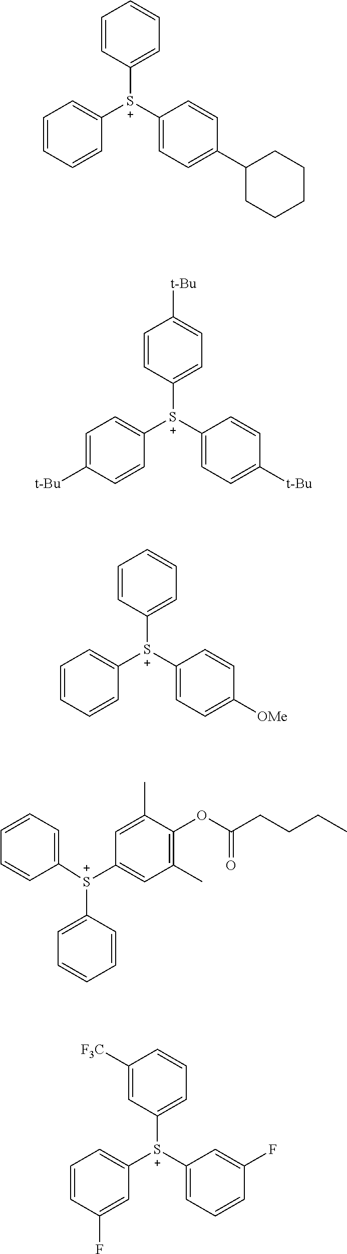 [SMILES: c1ccc([S+](c2ccccc2)c2ccc(C3CCCCC3)cc2)cc1 ; CC(C)(C)c1ccc([S+](c2ccc(C(C)(C)C)cc2)c2ccc(C(C)(C)C)cc2)cc1 ; COc1ccc([S+](c2ccccc2)c2ccccc2)cc1 ; CCCCC(=O)Oc1c(C)cc([S+](c2ccccc2)c2ccccc2)cc1C ; Fc1cccc([S+](c2cccc(F)c2)c2cccc(C(F)(F)F)c2)c1]

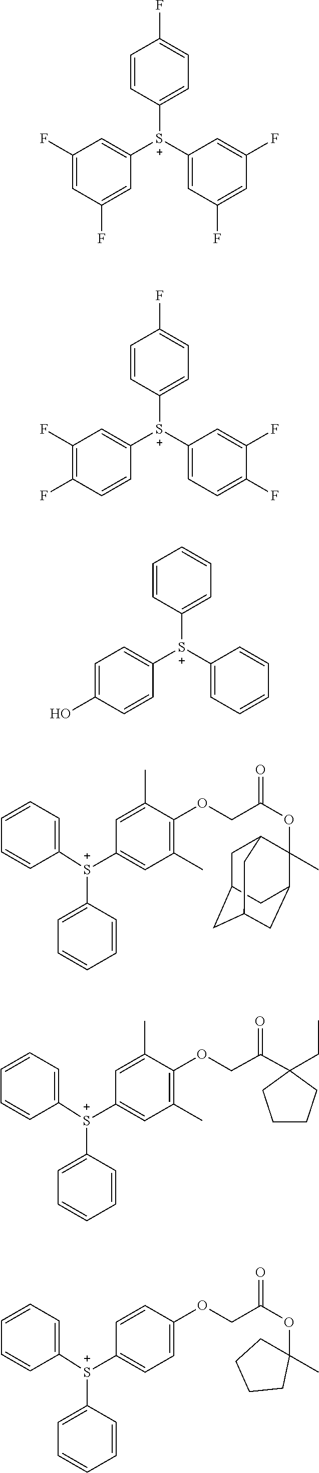 [SMILES: Fc1ccc([S+](c2cc(F)cc(F)c2)c2cc(F)cc(F)c2)cc1 ; Fc1ccc([S+](c2ccc(F)c(F)c2)c2ccc(F)c(F)c2)cc1 ; Oc1ccc([S+](c2ccccc2)c2ccccc2)cc1 ; Cc1cc([S+](c2ccccc2)c2ccccc2)cc(C)c1OCC(=O)OC1(C)C2CC3CC(C2)CC1C3 ; CCC1(C(=O)COc2c(C)cc([S+](c3ccccc3)c3ccccc3)cc2C)CCCC1 ; CC1(OC(=O)COc2ccc([S+](c3ccccc3)c3ccccc3)cc2)CCCC1]

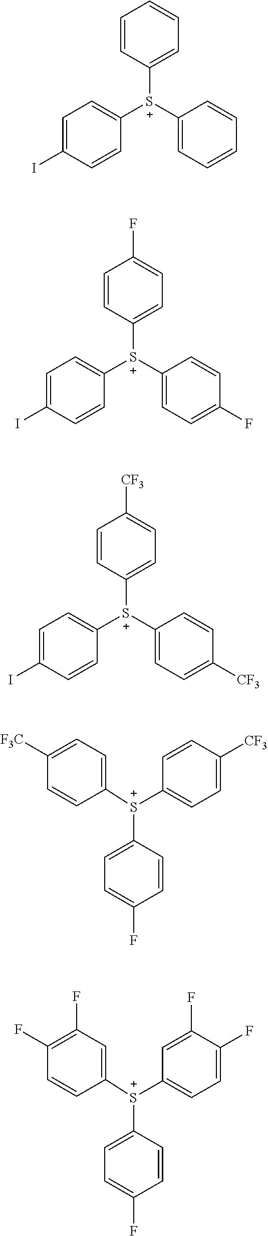 [SMILES: Ic1ccc([S+](c2ccccc2)c2ccccc2)cc1 ; Fc1ccc([S+](c2ccc(F)cc2)c2ccc(I)cc2)cc1 ; FC(F)(F)c1ccc([S+](c2ccc(I)cc2)c2ccc(C(F)(F)F)cc2)cc1 ; Fc1ccc([S+](c2ccc(C(F)(F)F)cc2)c2ccc(C(F)(F)F)cc2)cc1 ; Fc1ccc([S+](c2ccc(F)c(F)c2)c2ccc(F)c(F)c2)cc1]

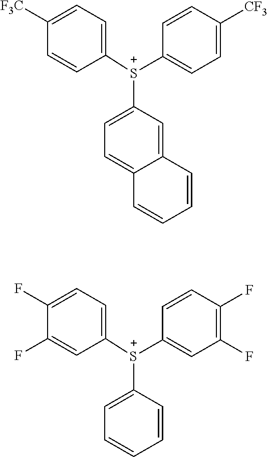 [SMILES: FC(F)(F)c1ccc([S+](c2ccc(C(F)(F)F)cc2)c2ccc3ccccc3c2)cc1 ; Fc1ccc([S+](c2ccccc2)c2ccc(F)c(F)c2)cc1F]

In the formula, tBu represents a t-butyl group, and Me represents a methyl group.

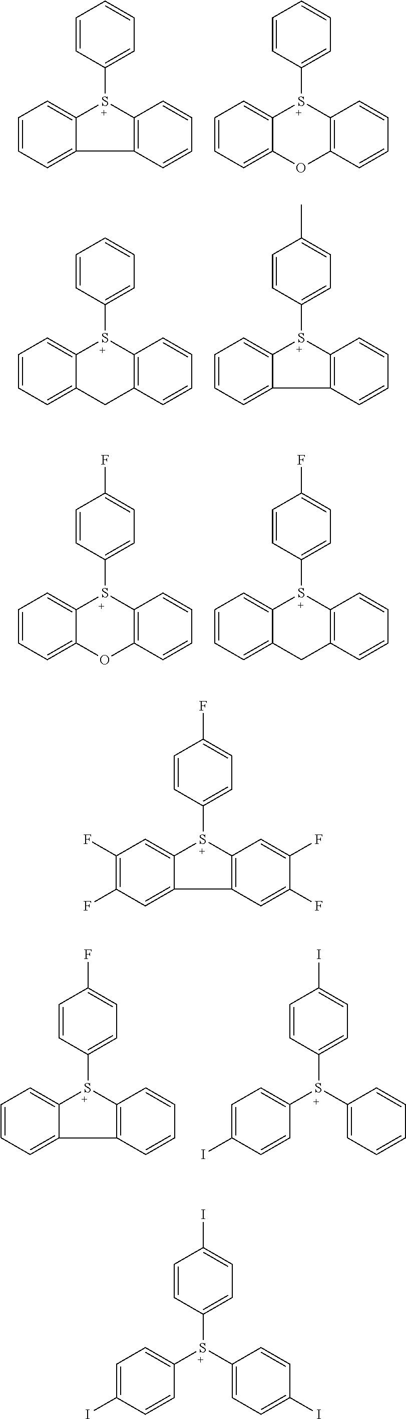

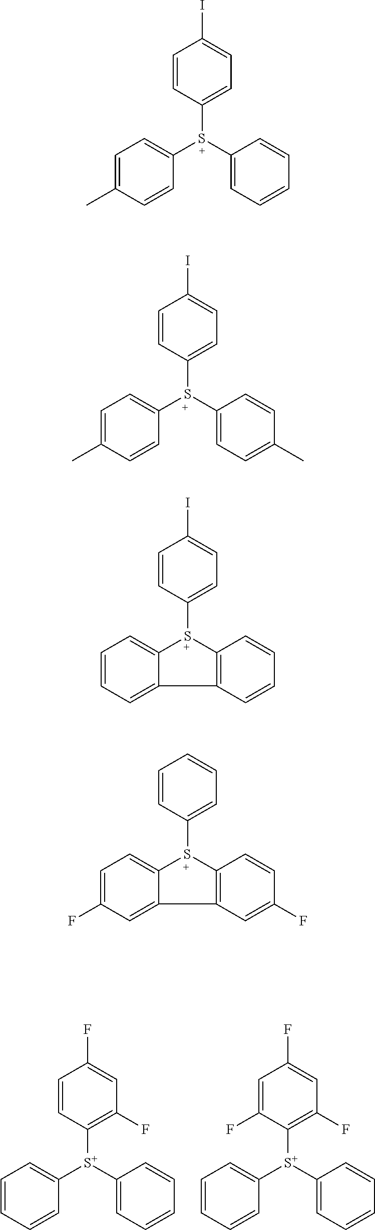

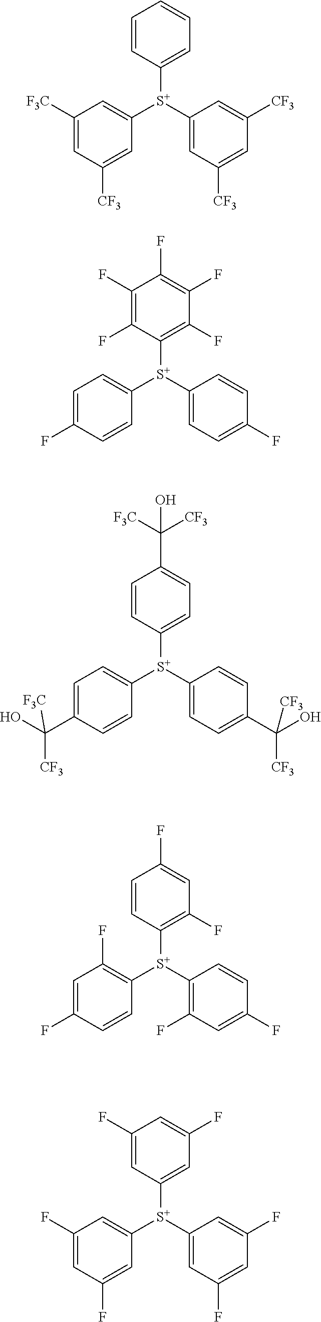

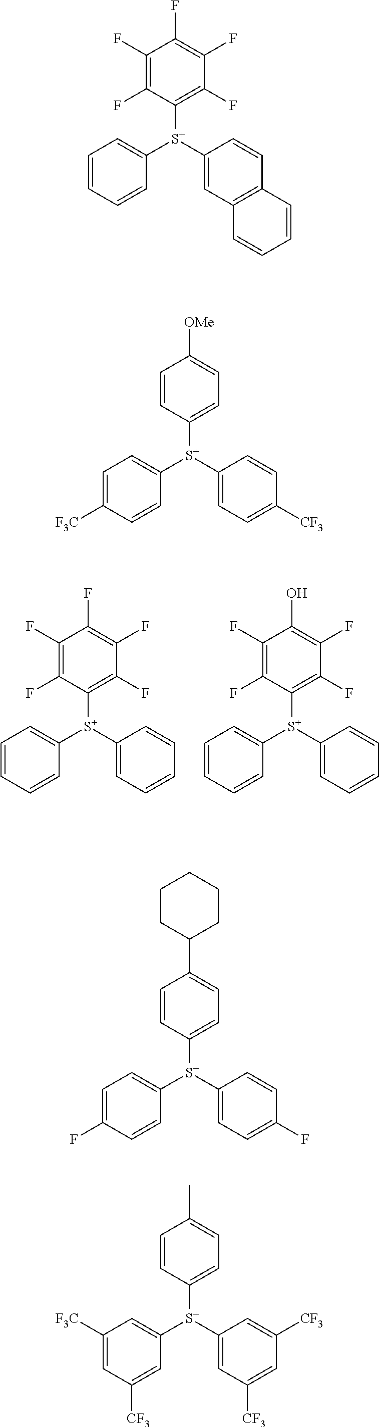

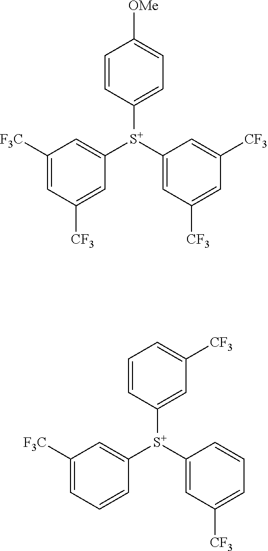

In the formula, Me represents a methyl group.

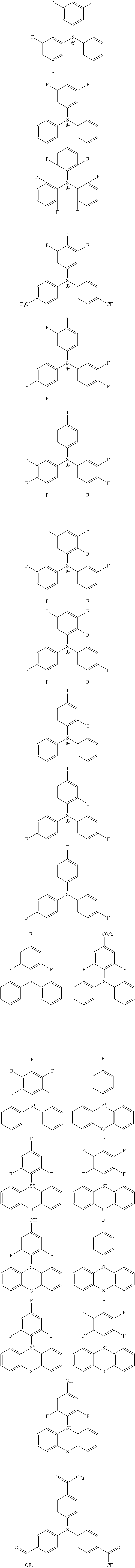

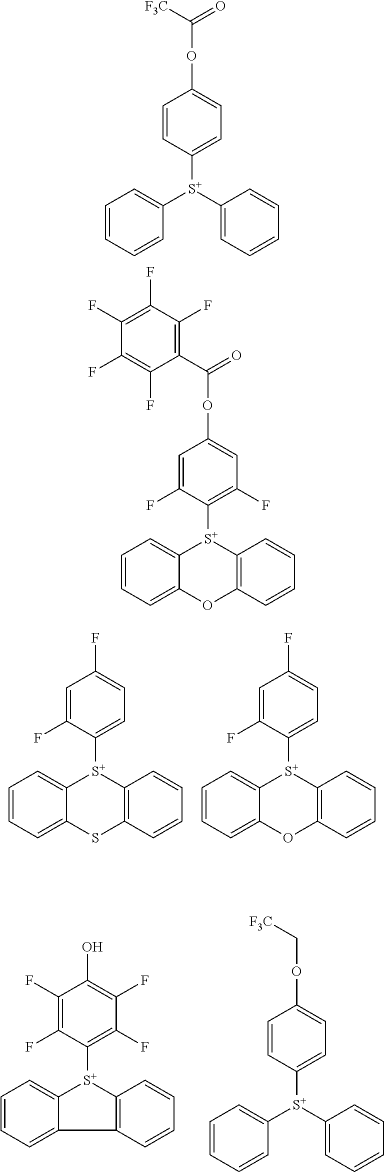

In the formula, Me represents a methyl group.

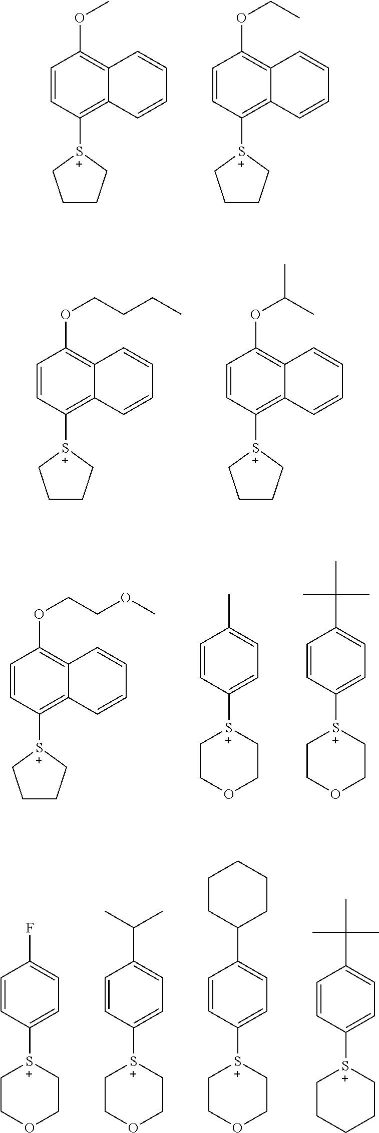

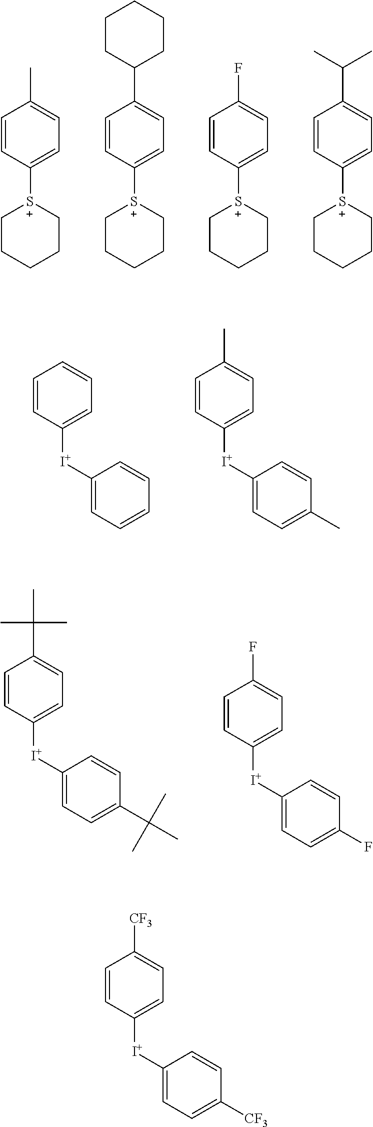

Any combination of the second organic acid anion and the second onium cation may be included in the radiation-sensitive acid generator (C). Among them, the following is preferable.

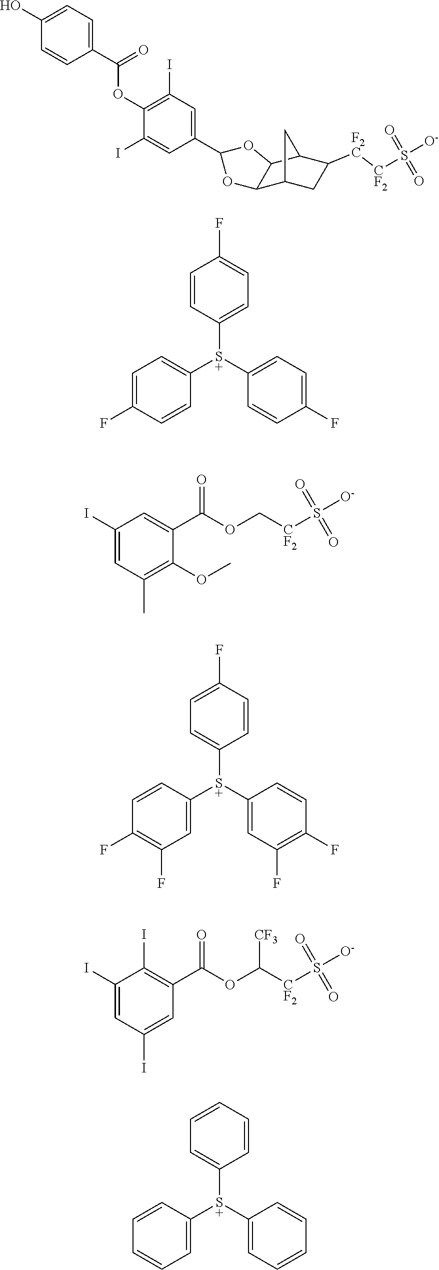 [SMILES: O=C(Oc1c(I)cc(C2OC3C4CC(C3O2)C(C(F)(F)C(F)(F)S(=O)(=O)[O-])C4)cc1I)c1ccc(O)cc1 ; Fc1ccc([S+](c2ccc(F)cc2)c2ccc(F)cc2)cc1 ; COc1c(C)cc(I)cc1C(=O)OCC(F)(F)S(=O)(=O)[O-] ; Fc1ccc([S+](c2ccc(F)c(F)c2)c2ccc(F)c(F)c2)cc1 ; O=C(OC(C(F)(F)F)C(F)(F)S(=O)(=O)[O-])c1cc(I)cc(I)c1I ; c1ccc([S+](c2ccccc2)c2ccccc2)cc1]

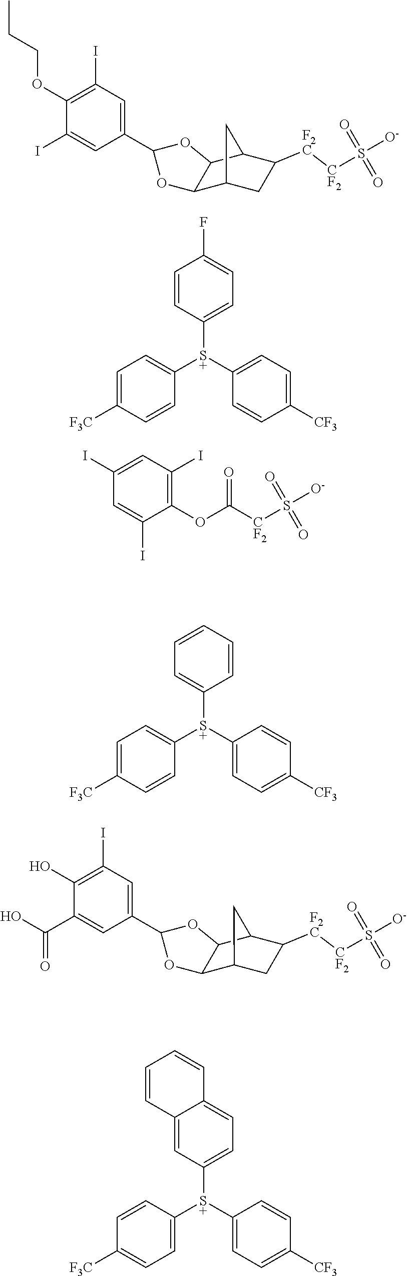 [SMILES: CCCOc1c(I)cc(C2OC3C4CC(C3O2)C(C(F)(F)C(F)(F)S(=O)(=O)[O-])C4)cc1I ; Fc1ccc([S+](c2ccc(C(F)(F)F)cc2)c2ccc(C(F)(F)F)cc2)cc1 ; O=C(Oc1c(I)cc(I)cc1I)C(F)(F)S(=O)(=O)[O-] ; FC(F)(F)c1ccc([S+](c2ccccc2)c2ccc(C(F)(F)F)cc2)cc1 ; O=C(O)c1cc(C2OC3C4CC(C3O2)C(C(F)(F)C(F)(F)S(=O)(=O)[O-])C4)cc(I)c1O ; FC(F)(F)c1ccc([S+](c2ccc(C(F)(F)F)cc2)c2ccc3ccccc3c2)cc1]

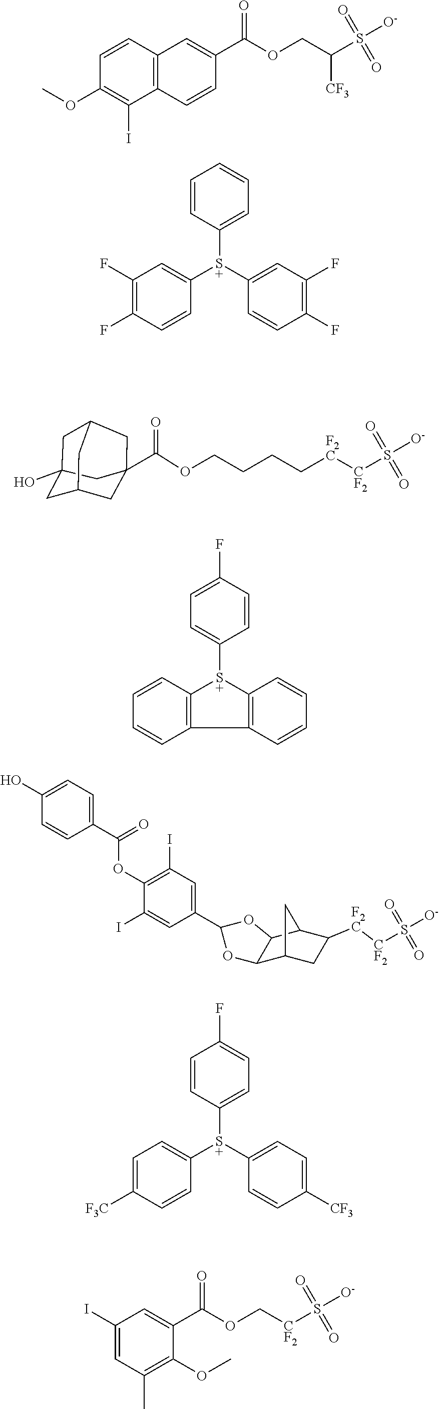

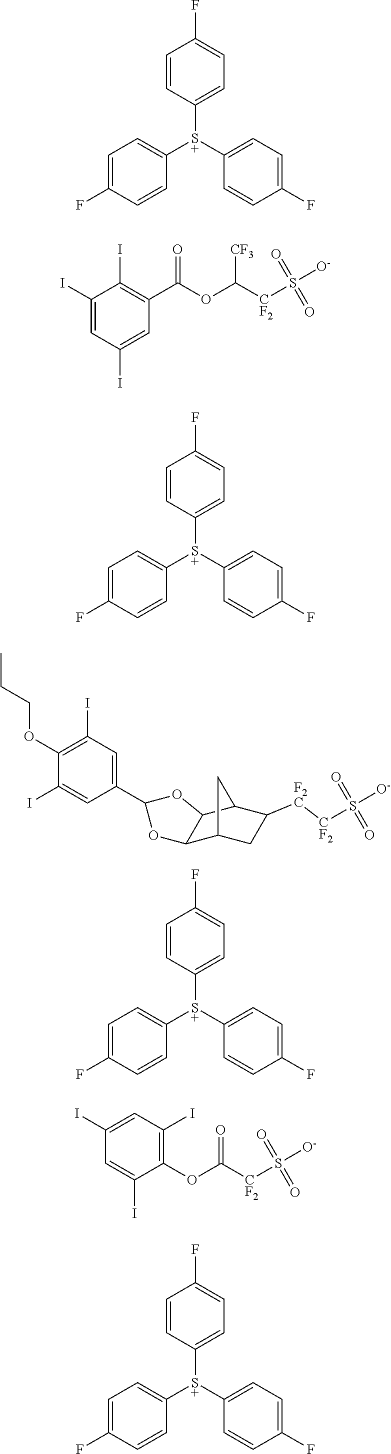

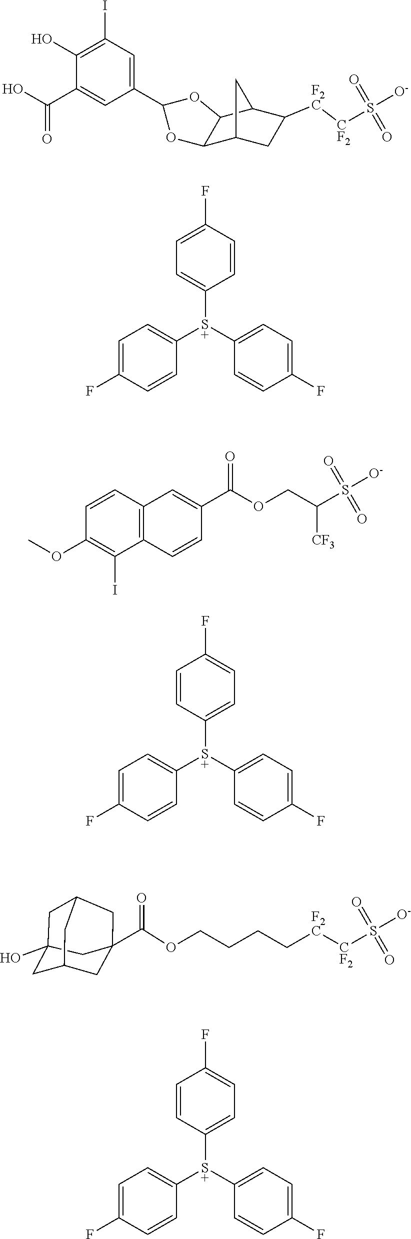 [SMILES: O=C(O)c1cc(C2OC3C4CC(C3O2)C(C(F)(F)C(F)(F)S(=O)(=O)[O-])C4)cc(I)c1O ; Fc1ccc([S+](c2ccc(F)cc2)c2ccc(F)cc2)cc1 ; COc1ccc2cc(C(=O)OCC(C(F)(F)F)S(=O)(=O)[O-])ccc2c1I ; Fc1ccc([S+](c2ccc(F)cc2)c2ccc(F)cc2)cc1 ; O=C(OCCCCC(F)(F)C(F)(F)S(=O)(=O)[O-])C12CC3CC(CC(O)(C3)C1)C2 ; Fc1ccc([S+](c2ccc(F)cc2)c2ccc(F)cc2)cc1]

The radiation-sensitive acid generator (C) can also be synthesized by a known method, particularly by a salt exchange reaction. A known radiation-sensitive acid generator other than the radiation-sensitive acid generator (C) may also be used in combination, as long as the effect of the present invention is not impaired.

These radiation-sensitive acid generator (C) may be used singly, or two or more thereof may be used in combination. When the radiation-sensitive composition contains a radiation-sensitive acid generator (C), the lower limit of the content (when a plurality of types is contained, the total content thereof is taken) of the radiation-sensitive acid generator (C) is preferably 10 parts by mass, more preferably 20 parts by mass, and still more preferably 30 parts by mass based on 100 parts by mass of the polymer (A). The upper limit of the content is preferably 80 parts by mass, more preferably 70 parts by mass, and still more preferably 65 parts by mass based on 100 parts by mass of the polymer (A). This makes it possible to exhibit superior sensitivity or CDU when forming a resist pattern.

When the polymer (A) is the radiation-sensitive acid-generating polymer (A1), the radiation-sensitive composition may contain the radiation-sensitive acid generator (B), or may not contain the radiation-sensitive acid generator (B).

### <Acid Diffusion Controlling Agent (D)>

The radiation-sensitive composition preferably contains an acid diffusion controlling agent (D) from the viewpoint of sensitivity and CDU. The acid diffusion controlling agent (D), which contains a third organic acid anion and a third onium cation, generates an acid having a pka higher than that of the acid generated from the radiation-sensitive acid generator (C) or the radiation-sensitive acid-generating polymer (A1) through irradiation with radiation. The acid diffusion controlling agent (D) containing the third organic acid anion and the third onium cation is preferably represented by the following formula (8-1) to the following formula (8-4).

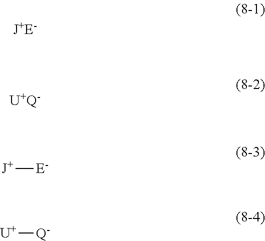

In the formulas (8-1) and (8-4), J⁺ is a sulfonium cation, and U⁺ is an iodonium cation. E⁻ and Q⁻ are each independently preferably at least one selected from the group consisting of R⁸SO₃⁻, R⁸COO⁻, and (R⁸SO₂)N⁻, more preferably R⁸COO⁻. In addition, a compound represented by the formula (8-3) containing a sulfonium cation and an anion in the same molecule and a compound represented by the formula (8-4) containing an iodonium cation and an anion in the same molecule are also included. The R⁸ is a monovalent organic group in the case of the formulas (8-1) and (8-2), and is a single bond in the case of the formulas (8-3) and (8-4).

The third organic acid anion is preferably an organic acid anion containing an iodine atom, and more preferably an organic acid anion having an iodine-substituted aromatic ring structure from the viewpoint of sensitivity.

Examples of the third organic acid anion include an anion represented by the following formulas. Among those shown below, as the structure having an iodine-substituted aromatic ring structure, a structure in which the iodine atom in the following formulas is substituted by an atom or a group other than an iodine atom such as a hydrogen atom or another substituent can also be suitably employed.

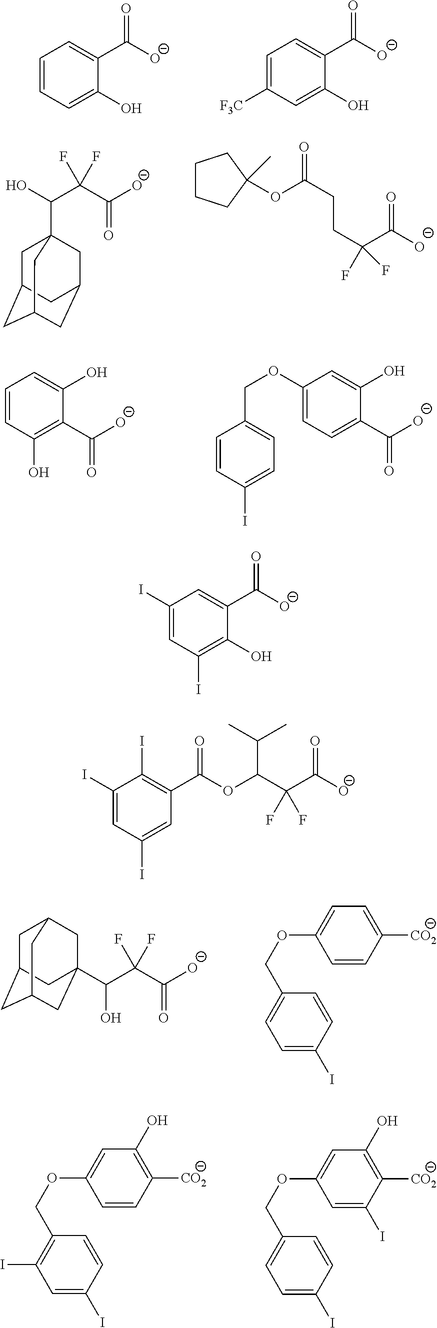

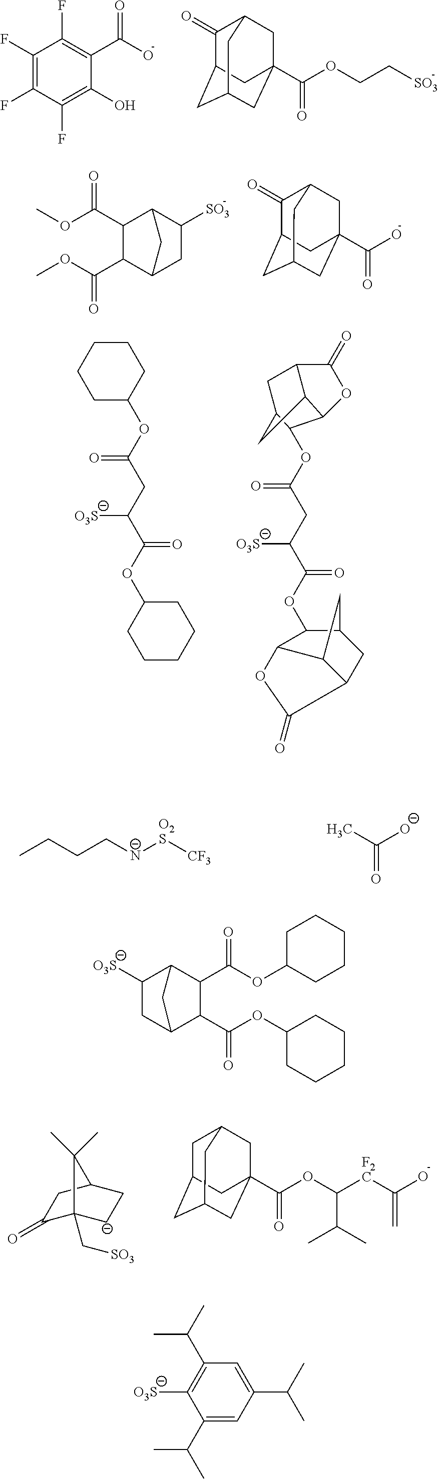

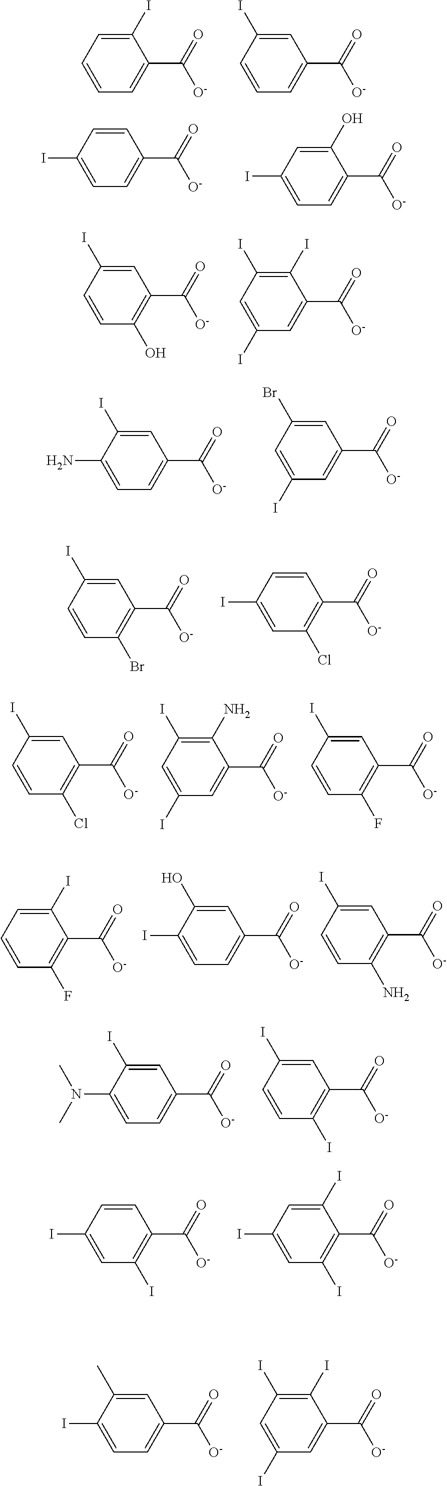

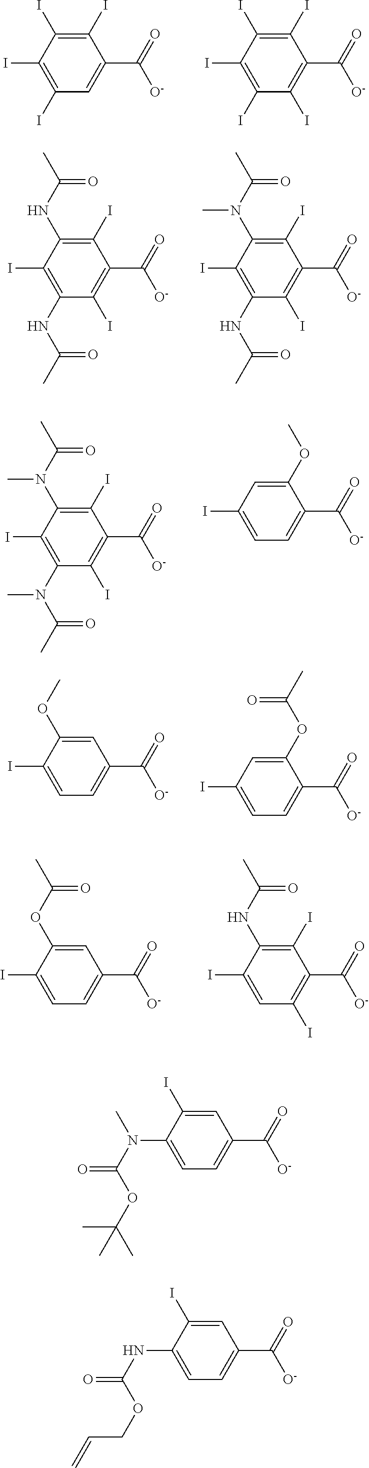

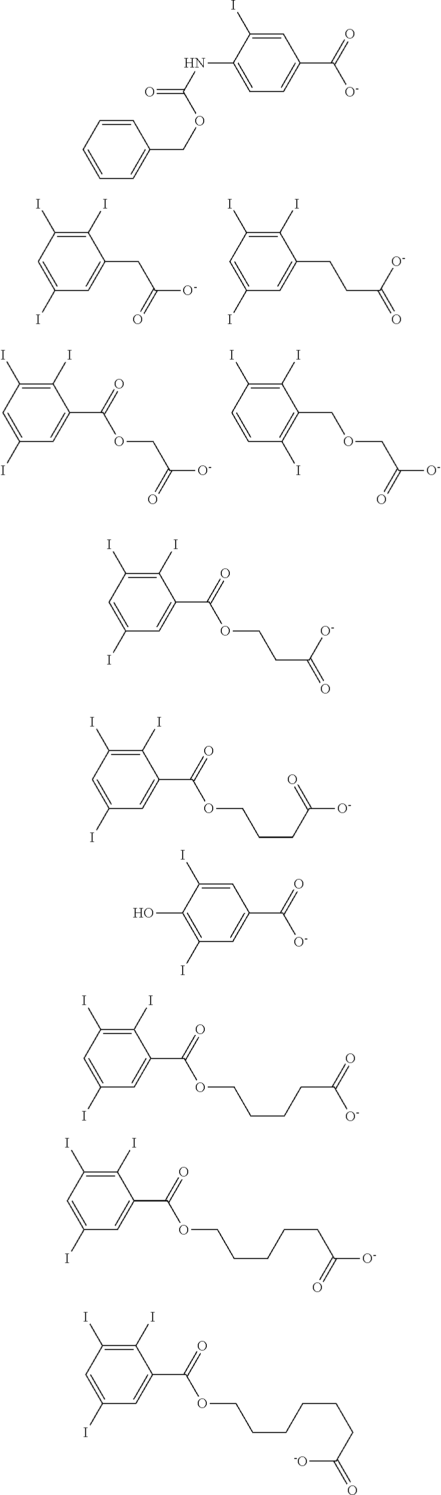

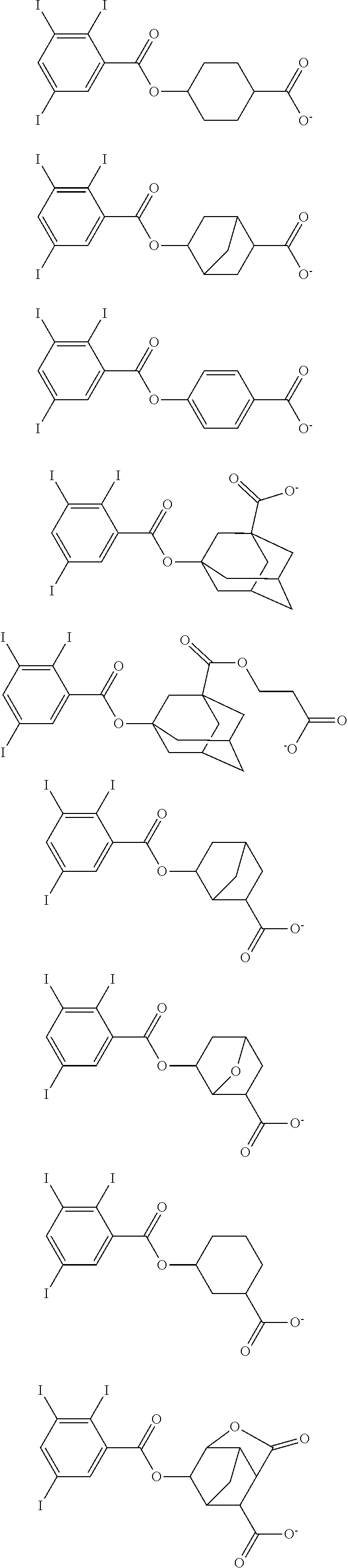

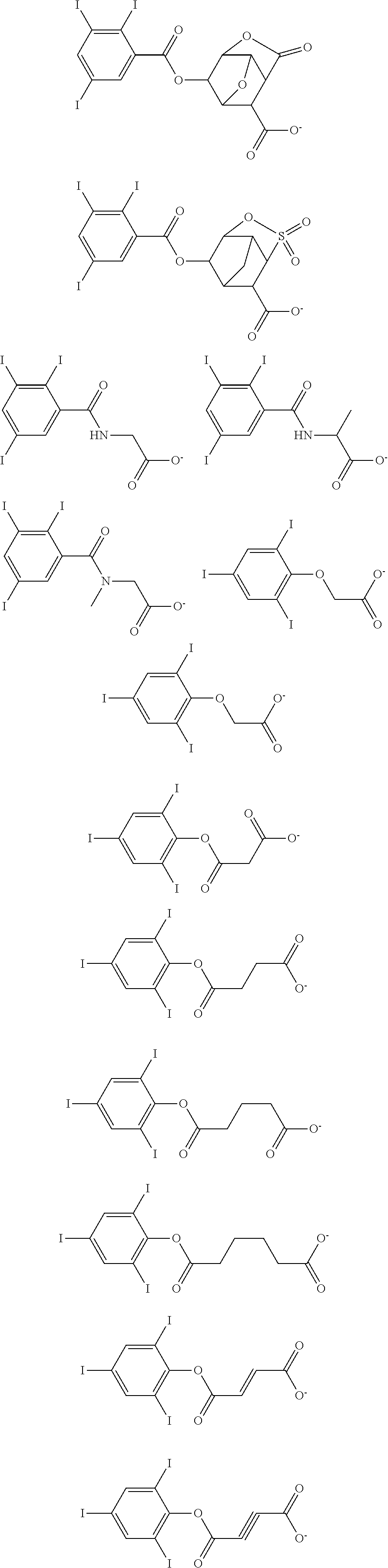

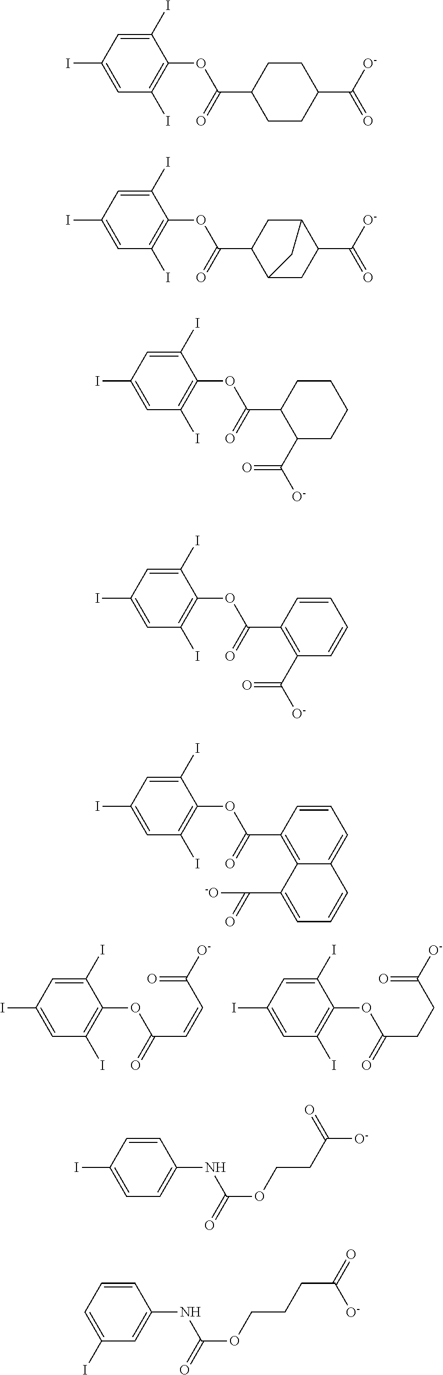

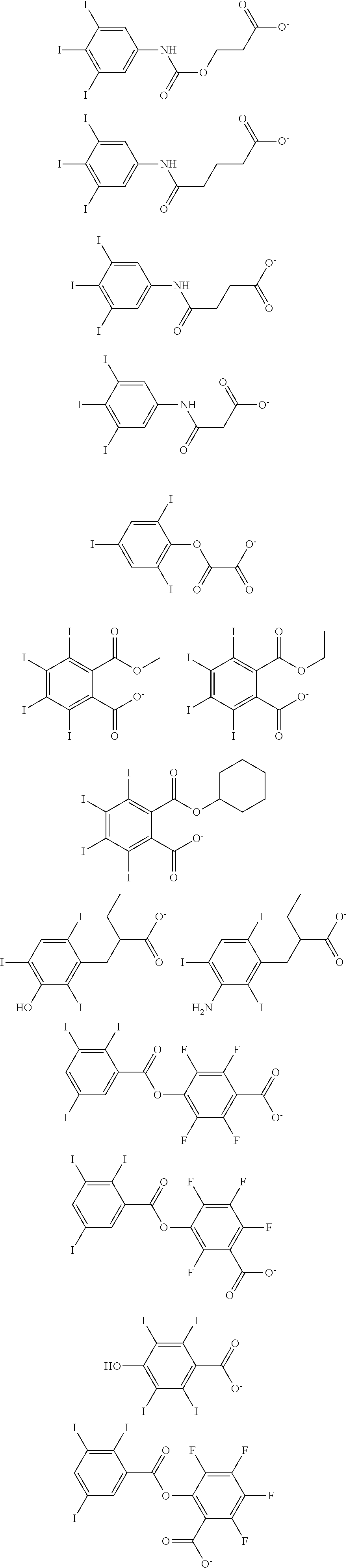

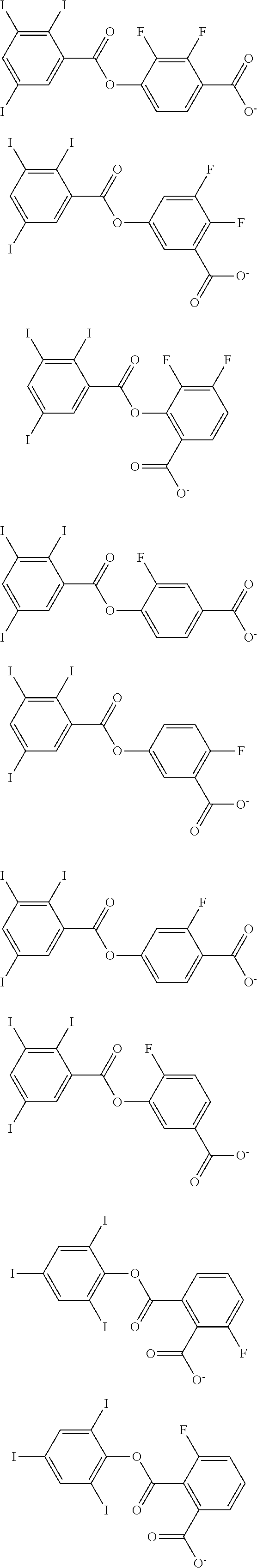

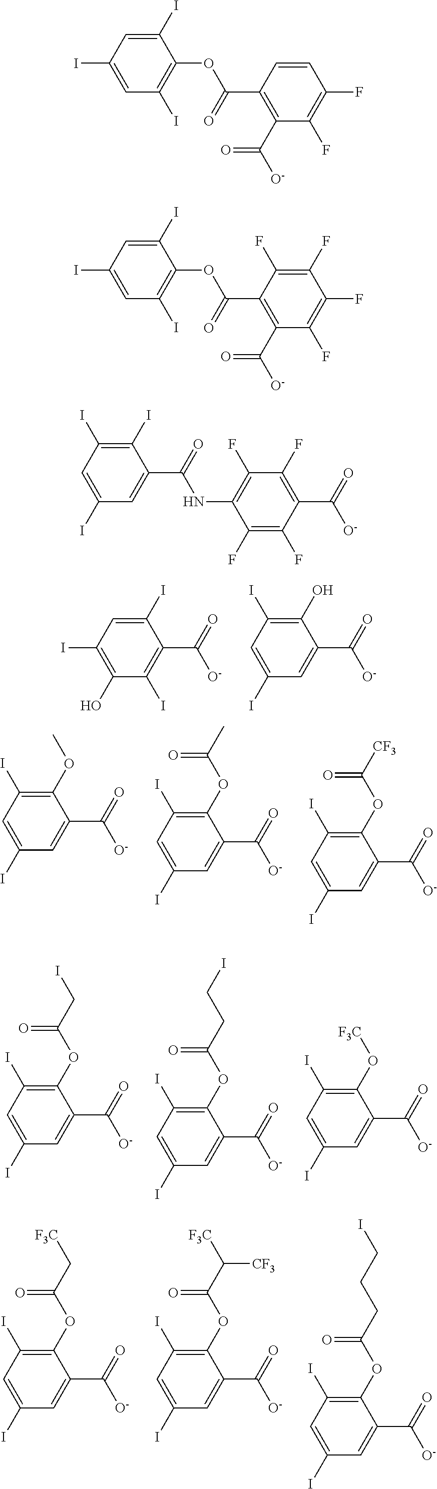

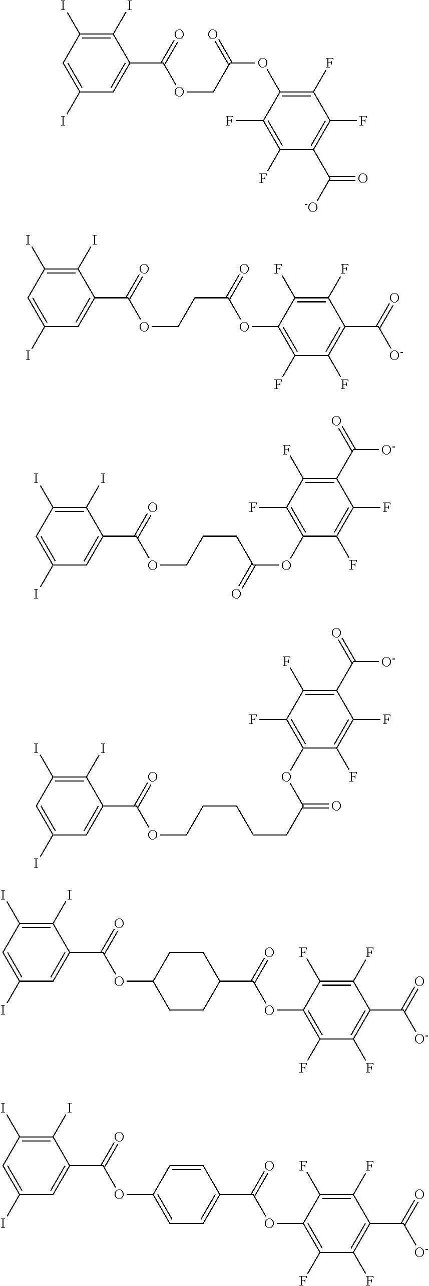

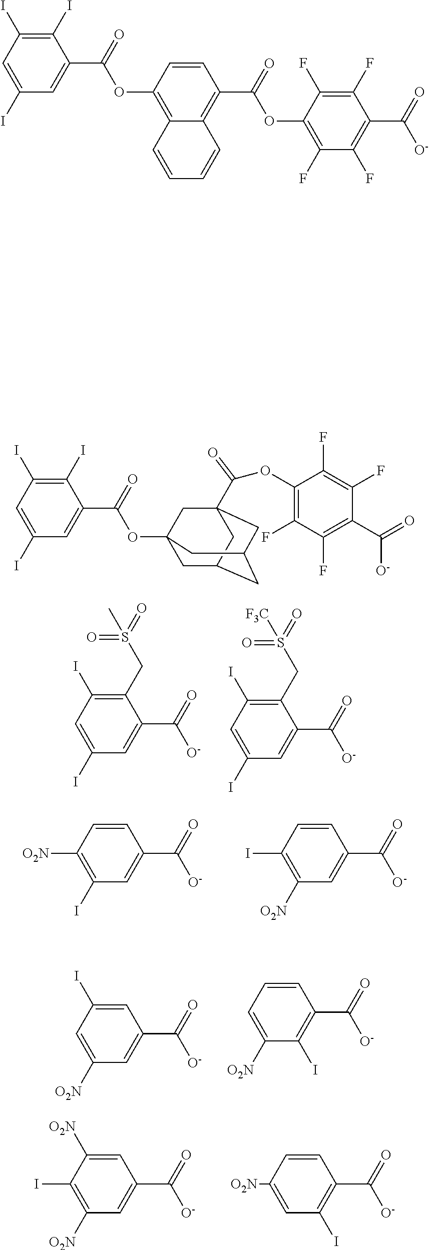

As the third onium cation, those exemplified as the second onium cation in the above radiation-sensitive acid generator (C) can be suitably employed.

The acid diffusion controlling agent (D) can be synthesized by a known method, particularly by a salt exchange reaction. A known acid diffusion controlling agent other than those described above may be used as long as the effect of the present invention is not impaired.

These acid diffusion controlling agents (D) may be used singly, or two or more thereof may be used in combination. The lower limit of the content of the acid diffusion controlling agent (when a plurality of types is contained, the total content thereof is taken) is preferably 30 mol %, more preferably 40 mol %, and still more preferably 45 mol % based on the radiation-sensitive acid generator (B) or the radiation-sensitive acid-generating polymer (A1) (when both are contained, the total content thereof is taken). The upper limit of the content is preferably 100 mol %, more preferably 90 mol %, and still more preferably 80 mol %. This makes it possible to exhibit superior sensitivity or CDU when forming a resist pattern.

### <Other Polymers>

The radiation-sensitive composition according to the present embodiment may contain, as another polymer, a polymer having higher content by mass of fluorine atoms than the above-described base polymer (hereinafter, also referred to as a “high fluorine-content polymer”). When the radiation-sensitive composition includes the high fluorine-content polymer, the high fluorine-content polymer can be localized in the surface layer of a resist film compared to the base polymer, and as a result, the state of the surface of the resist film and the component distribution in the resist film can be controlled to a desired state.

The high fluorine-content polymer preferably has a structural unit represented by the following formula (6) (hereinafter, also referred to as “structural unit (VI)”). In addition, for example, the high fluorine-containing polymer may, as necessary, have at least one of the structural units (I) to (V) in the base polymer.

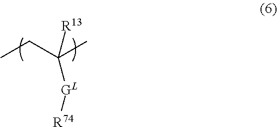

In the above formula (6), R⁷³ is a hydrogen atom, a methyl group, or a trifluoromethyl group. G^{L} is a single bond, an oxygen atom, a sulfur atom, —COO—, —SO₂ONH—, —CONH—, or —OCONH—. R⁷⁴ is a monovalent fluorine atom-containing organic group having 1 to 20 carbon atoms.

As R⁷³, a hydrogen atom and a methyl group are preferable from the viewpoint of the copolymerizability of a monomer that affords the structural unit (VI), and a methyl group is more preferable.

As the G^{L}, a single bond and —COO— are preferable from the viewpoint of the copolymerizability of a monomer that affords the structural unit (VI), and —COO— is more preferable.

Examples of the monovalent fluorine atom-containing organic group having 1 to 20 carbon atoms represented by R⁷⁴ may include a monovalent fluorine atom-containing organic group in which part or all of the hydrogen atoms of the monovalent organic group having 1 to 20 carbon atoms represented by R² in the above formula (1) are substituted with fluorine atoms can be suitably mentioned.

When the high fluorine-content polymer has the structural unit (VI), the lower limit of the content of the structural unit (VI) is preferably 50 mol %, more preferably 60 molo, and still more preferably 65 mol % based on the total amount of all structural units constituting the high fluorine-content polymer. The upper limit of the content is preferably 100 mol %, more preferably 95 mol %, and still more preferably 90 mol %. When the content of the structural unit (VI) is adjusted to within the above range, the mass content of fluorine atoms in the high fluorine-content polymer can more appropriately be adjusted and the localization in the surface layer of a resist film can be further promoted.

The lower limit of the Mw of the high fluorine-content polymer is preferably 1,000, more preferably 2,000, still more preferably 3,000, and particularly preferably 5,000. The upper limit of the Mw is preferably 50, 000, more preferably 30, 000, still more preferably 20,000, and particularly preferably 15,000.

The Mw/Mn of the high fluorine-content polymer is usually 1 or more, and preferably 1.1 or more. The Mw/Mn is usually 5 or less, preferably 3 or less, more preferably 2.5 or less, and still more preferably 2.2 or less.

The lower limit of the content of the high fluorine-content polymer is preferably 0.5 parts by mass, more preferably 1 part by mass, and still more preferably 2 parts by mass based on 100 parts by mass of the polymer (A). The upper limit of the content is preferably 10 parts by mass, more preferably 8 parts by mass, and still more preferably 5 parts by mass. When the content of the high fluorine-content polymer is adjusted to within the above range, the high fluorine-content polymer can be more effectively localized in the surface layer of a resist film, and as a result, the elusion of a top portion of the pattern is suppressed during development, and the CDU and the like can be enhanced. The radiation-sensitive composition may contain one type or two or more types of high fluorine-content polymer.

### (Method for Synthesizing High Fluorine-Content Polymer)

The high fluorine-content polymer can be synthesized by the same method as the method for synthesizing the base polymer described above.

### <Solvent (B)>

The radiation-sensitive composition according to the present embodiment contains a solvent (B). The solvent (B) is not particularly limited as long as it is a solvent capable of dissolving or dispersing the polymer (A), additives contained as desired, and the like.

Examples of the solvent (B) include an alcohol-based solvent, an ether-based solvent, a ketone-based solvent, an amide-based solvent, an ester-based solvent, and a hydrocarbon-based solvent.

Examples of the alcohol-based solvent include:
monoalcohol-based solvents having 1 to 18 carbon atoms, such as iso-propanol, 4-methyl-2-pentanol, 3-methoxybutanol, n-hexanol, 2-ethylhexanol, furfuryl alcohol, cyclohexanol, 3, 3, 5-trimethylcyclohexanol, diacetone alcohol and methyl 2-hydroxyisobutyrate;
polyhydric alcohol-based solvents having 2 to 18 carbon atoms, such as ethylene glycol, 1,2-propylene glycol, 2-methyl-2, 4-pentanediol, 2, 5-hexanediol, diethylene glycol, dipropylene glycol, triethylene glycol, and tripropylene glycol; and
partially etherized polyhydric alcohol-based solvents obtained by etherifying some of hydroxy groups of the polyhydric alcohol-based solvent.

In the present embodiment, alcohol acid ester-based solvents such as methyl lactate, ethyl lactate, propyl lactate, butyl lactate, methyl 2-hydroxyisobutyrate, i-propyl 2-hydroxyisobutyrate, i-butyl 2-hydroxyisobutyrate, and n-butyl 2-hydroxyisobutyrate are also included in the alcohol-based solvent.

Examples of the ether-based solvent include:
dialkyl ether-based solvents, such as diethyl ether, dipropyl ether, and dibutyl ether;
cyclic ether-based solvents, such as tetrahydrofuran and tetrahydropyran;
aromatic ring-containing ether-based solvents, such as diphenyl ether and anisole (methyl phenyl ether); and
etherized polyhydric alcohol-based solvents obtained by etherifying a hydroxy group of the polyhydric alcohol-based solvent.

Examples of the ketone-based solvent include:
chain ketone-based solvents, such as acetone, butanone, and methyl-iso-butyl ketone;
cyclic ketone-based solvents, such as cyclopentanone, cyclohexanone, and methylcyclohexanone; and
2, 4-pentanedione, acetonylacetone, and acetophenone.

Examples of the amide-based solvent include:
cyclic amide-based solvents, such as N,N′-dimethylimidazolidinone and N-methylpyrrolidone; and
chain amide-based solvents, such as N-methylformamide, N, N-dimethylformamide, N, N-diethylformamide, acetamide, N-methylacetamide, N, N-dimethylacetamide, and N-methylpropionamide.

Examples of the ester-based solvent include:
monocarboxylic acid ester-based solvents, such as n-butyl acetate;
partially etherized polyhydric alcohol acetate-based solvents, such as diethylene glycol mono-n-butyl ether acetate, propylene glycol monomethyl ether acetate, and dipropylene glycol monomethyl ether acetate;
lactone-based solvents, such as γ-butyrolactone and valerolactone;
carbonate-based solvents, such as diethyl carbonate, ethylene carbonate, and propylene carbonate; and
polyvalent carboxylic acid diester-based solvents, such as propylene glycol diacetate, methoxytriglycol acetate, diethyl oxalate, ethyl acetoacetate, and diethyl phthalate.

Examples of the hydrocarbon-based solvent include:
aliphatic hydrocarbon-based solvents, such as n-hexane, cyclohexane, and methylcyclohexane; and
aromatic hydrocarbon-based solvents, such as benzene, toluene, di-iso-propylbenzene, and n-amylnaphthalene.

Among them, an ester-based solvent, a ketone-based solvent, an alcohol-based solvent, and an ether-based solvent are preferable, a partially etherized polyhydric alcohol acetate-based solvent, a cyclic ketone-based solvent, a lactone-based solvent, a alcohol acid ester-based solvent, a partially etherized polyhydric alcohol-based solvent, and a monocarboxylic acid ester-based solvent are more preferable, and propylene glycol monomethyl ether acetate, cyclohexanone, Y-butyrolactone, propylene glycol monomethyl ether, diacetone alcohol, and ethyl lactate are still more preferable, propylene glycol monomethyl ether acetate and propylene glycol monomethyl ether are particularly preferable. The radiation-sensitive composition may contain one type or two or more types of solvent.

### <Other Optional Components>

The radiation-sensitive composition may contain, in addition to the components, other optional components. Examples of other optional components may include a cross-linking agent, a localization enhancing agent, a surfactant, an alicyclic backbone-containing compound, and a sensitizer. These other optional components may be used singly, or two or more thereof may be used in combination.

### <Method for Preparing Radiation-Sensitive Composition>

The radiation-sensitive composition can be prepared, for example, by mixing the polymer (A) and the solvent (B), and if necessary, the optional component at a prescribed ratio. The radiation-sensitive composition is, after the mixing, preferably filtered through, for example, a filter having a pore size of approximately 0.05 μm to 0.4 μm. The solid matter concentration of the radiation-sensitive composition is usually 0.1% by mass to 50% by mass, preferably 0.5% by mass to 30% by mass, more preferably 18 by mass to 20% by mass.

### <<Pattern Forming Method>>

A pattern forming method according to the present embodiment includes:
a step (1) of applying the radiation-sensitive composition directly or indirectly on a substrate to form a resist film (hereinafter also referred to as “resist film forming step”);
a step (2) of exposing the resist film to light (hereinafter also referred to as “exposure step”); and
a step (3) of developing the exposed resist film with a developer (hereinafter also referred to as “development step”).

According to the pattern forming method, the radiation-sensitive composition capable of forming a resist film excellent in sensitivity, CDU and development defect-suppressing properties is used, and therefore a high-quality resist pattern can be formed. Hereinbelow, each of the steps will be described.

### [Resist Film Forming Step]

In this step (the step (1)), a resist film is formed from the radiation-sensitive composition. Examples of the substrate on which the resist film is formed may include those traditionally known in the art, including a silicon wafer, silicon dioxide, and a wafer coated with aluminum. An organic or inorganic antireflective film disclosed in, for example, JP-B-6-12452 or JP-A-59-93448 may be formed on the substrate. Examples of an applicating method may include a rotary coating (spin coating), flow casting, and roll coating. After the application, prebaking (PB) may be performed to volatilize the solvent in the coating film, as necessary. The temperature of PB is usually from 60° C. to 160° C., preferably from 80° C. to 140° C. The duration of PB is usually from 5 seconds to 600 seconds, preferably from 10 seconds to 300 seconds. The thickness of the resist film formed is preferably from 10 nm to 1, 000 nm, more preferably from 10 nm to 500 nm.

When the subsequent exposure step is performed with radiation having a wavelength of 50 nm or less, it is preferable to use a polymer having the structural unit (II) as the base polymer in the composition.

### [Exposure Step]

In this step (the step (2)), the resist film formed in the resist film forming step as the step (1) is exposed by irradiating with radiation through a photomask (possibly through an immersion medium such as water). Examples of the radiation to be used for the exposure may include an electromagnetic wave including visible ray, ultraviolet ray, far ultraviolet ray, extreme ultraviolet ray (EUV), X ray, and γ ray; and charged particle radiation such as an electron beam and x ray, depending on the desired line width of the pattern. Among them, far ultraviolet ray, an electron beam, or EUV is preferable. ArF excimer laser light (wavelength is 193 nm), KrF excimer laser light (wavelength is 248 nm), an electron beam, or EUV is more preferable. An electron beam having a wavelength of 50 nm or less and EUV, which are identified as the next generation exposing technologies, are further preferable.

After the exposure, post exposure bake (PEB) is preferably performed to promote the dissociation of the acid-dissociable group of the polymer by an acid generated from the radiation-sensitive acid generator through exposure to light in the exposed part of the resist film. As a result of the PEB, there is generated a difference in solubility into a developer between the exposed area and the unexposed area. The temperature of PEB is usually from 50° C. to 180° C., preferably from 80° C. to 150° C. The duration of PEB is usually from 5 seconds to 600 seconds, preferably from 10 seconds to 300 seconds.

### [Development Step]

In this step (the step (3)), the resist film exposed in the exposure step as the step (2) is developed with a developer. By this step, the predetermined resist pattern can be formed. After the development, the resist pattern is generally washed with a rinse solution such as water or alcohol, and then dried.

Examples of the developer used for the development may include, in the alkaline development, an alkaline aqueous solution obtained by dissolving at least one alkaline compound such as sodium hydroxide, potassium hydroxide, sodium carbonate, sodium silicate, sodium metasilicate, ammonia water, ethylamine, n-propylamine, diethylamine, di-n-propylamine, triethylamine, methyldiethylamine, ethyldimethylamine, triethanolamine, tetramethyl ammonium hydroxide (TMAH), pyrrole, piperidine, choline, 1,8-diazabicyclo-[5.4.0]-7-undecene, 1,5-diazabicyclo-[4.3.0]-5-nonene. Among them, an aqueous TMAH solution is preferable, and a 2.38% by mass aqueous TMAH solution is more preferable.

In the case of organic solvent development, examples of the solvent may include organic solvents such as hydrocarbon-based solvents, ether-based solvents, ester-based solvents, ketone-based solvents, and alcohol-based solvents, and solvents containing an organic solvent. Examples of the organic solvent may include one, two or more solvents listed as the solvent for the radiation-sensitive composition. Among them, ester-based solvents and ketone-based solvents are preferable. As the ester-based solvents, acetate-based solvents are preferable, and n-butyl acetate and amyl acetate are more preferable. As the ketone-based solvents, chain ketones are preferable, and 2-heptanone is more preferable. The content of the organic solvent in a developer is preferably 80% by mass or more, more preferably 90% by mass or more, still more preferably 95% by mass or more, particularly preferably 99% by mass or more. Examples of the component other than the organic solvent in the developer may include water and silicone oil.

Examples of the developing method may include a method including dipping a substrate in a tank filled with a developer for a given time (dipping method); a developing method including raising a developer on the surface of a substrate due to surface tension and leaving the raised developer for a given time (paddling method); a method including spraying a developer on the surface of a substrate (spraying method); and a method including injecting a developer on a substrate rolling at a constant rate while scanning a developer injection nozzle at a constant rate (dynamic dispensing method).

### <<Polymer>>

The polymer according to the present embodiment contains a structural unit (I) represented by the following formula (1).

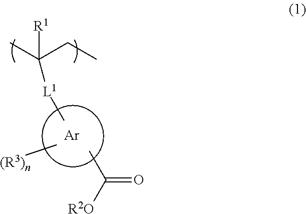

wherein in the formula (1),
R¹ is a hydrogen atom, a fluorine atom, a methyl group, or a trifluoromethyl group;
L¹ is a single bond or a divalent linking group;
Ar represents a furan ring, a thiophene ring, or an aromatic heterocycle having 9 to 20 ring members containing a furan ring or a thiophene ring;
R² is a hydrogen atom or a monovalent organic group having 1 to 20 carbon atoms;
R³ is a halogen atom, a hydroxy group, a cyano group, a nitro group, an amino group, a thiol group, or a monovalent organic group having 1 to 20 carbon atoms;
n is an integer of 0 to 4; and when n is 2 or more, a plurality of R³s are identical to or different from each other.

The structural unit (I) is as described above. It is only required that the polymer contains the structural unit (I). The polymer may also contain a structural unit other than the structural unit (I). Specifically, the polymer can contain the structural units (II) to (V). Examples of such a polymer include the polymer (A) or polymer (A1).

### <<Compound>>

The compounds according to the present embodiment relate to the following Compounds 1 to 5.

### <Compound 1>

Compound 1 is represented by the following formula (1-1).

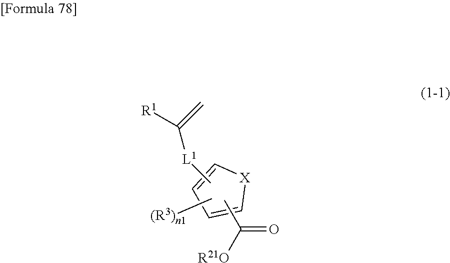

wherein in the formula (1-1),
X represents —S— or —O—;
R¹ is a hydrogen atom, a fluorine atom, a methyl group, or a trifluoromethyl group;
L¹ is a single bond or a divalent linking group;
R²¹ is an acid-dissociable group represented by a following formula (2-1), provided that when X is —S—, a total number of carbon atoms of L¹ and R²¹ is 5 or more:

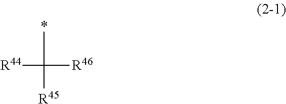

wherein in the formula (2-1),
R⁴⁴ is a hydrogen atom or a monovalent organic group having 1 to 10 carbon atoms;
R⁴⁵ and R⁴⁶ each independently represent a monovalent organic group having 1 to 10 carbon atoms, or a divalent alicyclic group or heterocyclic group having 3 to 10 ring members composed of R⁴⁵ and R⁴⁶ combined with each other together with a carbon atom to which R⁴⁵ and R⁴⁶ are bonded,
provided that when R⁴⁴, R⁴⁵, or R⁴⁶ has a double bond other than an aromatic ring, two carbon atoms composing the double bond are bonded to 0, 2, or 3 hydrogen atoms in total; and
* represents a bond bonded to an oxygen atom,
R³ is a halogen atom, a hydroxy group, a cyano group, a nitro group, an amino group, a thiol group, or a monovalent organic group having 1 to 20 carbon atoms;
n1 is an integer of 0 to 2; and when n1 is 2, two R³s are identical to or different from each other.
R¹, L¹, and R³ have the same meaning as R¹, L¹, and R³ in the formula (1).
R²¹ is an acid-dissociable group represented by the formula (2-1), and when X is —S—, the total number of carbon atoms of L¹ and R²¹ is 5 or more, preferably 7 or more.

As the monovalent organic group having 1 to 10 carbon atoms represented by R⁴⁴, a monovalent organic group having 1 to 10 carbon atoms represented by R⁴¹ in the formula (2) can be suitably employed.

As the monovalent organic group having 1 to 10 carbon atoms represented by R⁴⁵ and R⁴⁶, and the divalent alicyclic group or heterocyclic group having 3 to 10 ring members composed of R⁴⁵ and R⁴⁶ combined with each other together with a carbon atom to which R⁴⁵ and R⁴⁶ are bonded, a monovalent organic group having 1 to 10 carbon atoms represented by R⁴² and R⁴³ in the formula (2) and a divalent alicyclic group or heterocyclic group having 3 to 10 ring members composed of R⁴² and R⁴³ combined with each other together with a carbon atom to which R⁴² and R⁴³ are bonded can be suitably employed.

When R⁴⁴, R⁴⁵, or R⁴⁶ has a double bond other than an aromatic ring, two carbon atoms composing the double bond are bonded to 0, 2, or 3 hydrogen atoms in total, preferably bonded to 3 hydrogen atoms.

Examples of Compound 1 represented by the formula (1-1) include the following compounds (m1-1) to (m1-17).

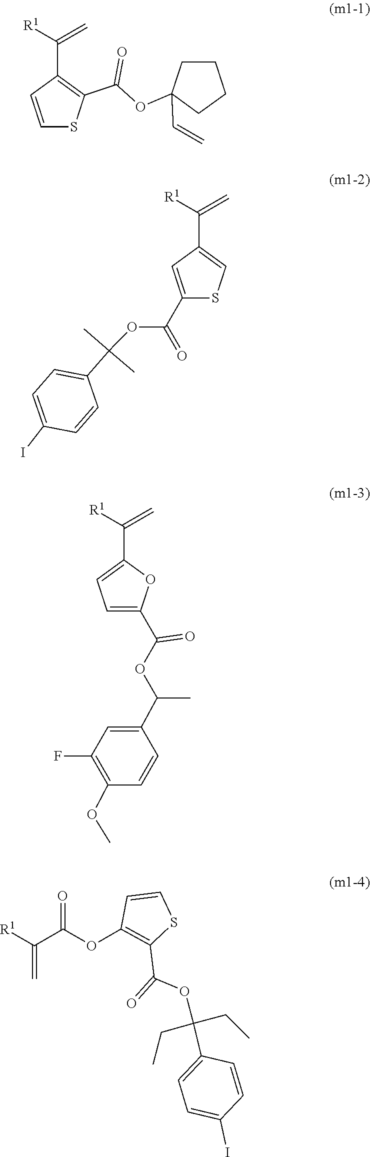

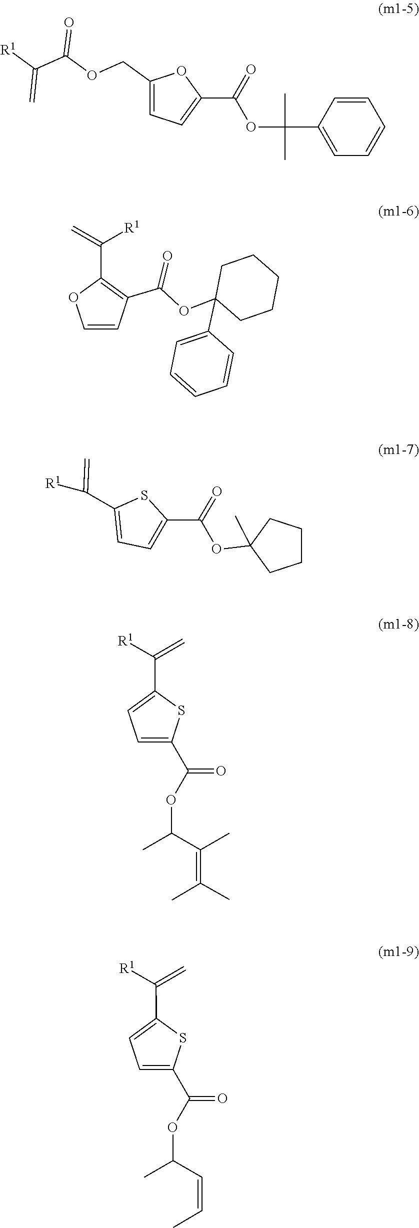

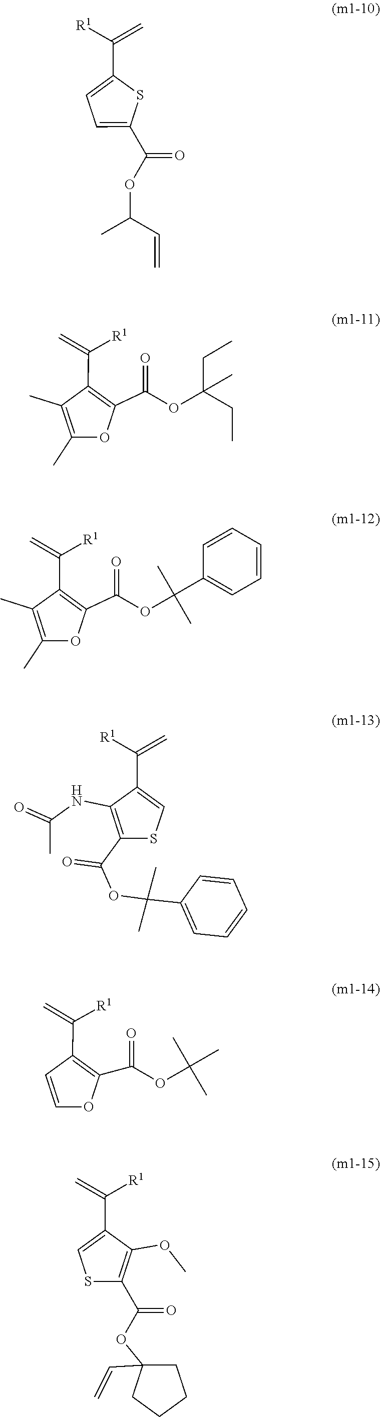

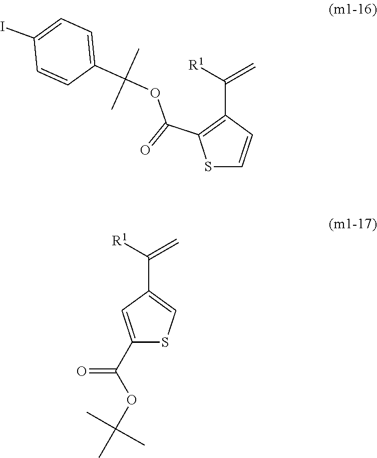

In the formula, R¹ has the same meanings as in the formula (1-1).

### <Compound 2>

Compound 2 is represented by the following formula (1-2-1) or the following formula (1-2-2).

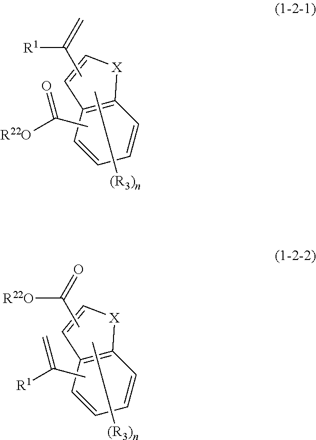

wherein in the formula (1-2-1) and the formula (1-2-2),
X represents —S— or —O—;
R¹ is a hydrogen atom, a fluorine atom, a methyl group, or a trifluoromethyl group;
R²² is an acid-dissociable group represented by a following formula (2-2), provided that a number of carbon atoms of R²² is 6 or more:

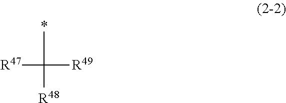

wherein in the formula (2-2),
R⁴⁷ is a hydrogen atom or a monovalent organic group having 1 to 10 carbon atoms;
R⁴⁸ and R⁴⁹ each independently represent a monovalent organic group having 1 to 10 carbon atoms, or a divalent alicyclic group or heterocyclic group having 3 to 10 ring members composed of R⁴⁸ and R⁴⁹ combined with each other together with a carbon atom to which R⁴⁸ and R⁴⁹ are bonded,
provided that when R⁴⁷, R⁴⁸, or R⁴⁹ has a double bond other than an aromatic ring, two carbon atoms composing the double bond are bonded to 0, 1, or 3 hydrogen atoms in total; and
* represents a bond bonded to an oxygen atom,
R³ is a halogen atom, a hydroxy group, a cyano group, a nitro group, an amino group, a thiol group, or a monovalent organic group having 1 to 20 carbon atoms;
n is an integer of 0 to 4; and when n is 2 or more, a plurality of R³s are identical to or different from each other.
R¹, R³, and n have the same meaning as R¹, R³, and n in the formula (1).
R²² is an acid-dissociable group represented by the formula (2-2), and the number of carbon atoms of R²² is 6 or more, preferably 7 or more.

As the monovalent organic group having 1 to 10 carbon atoms represented by R⁴⁷, a monovalent organic group having 1 to 10 carbon atoms represented by R⁴¹ in the formula (2) can be suitably employed.

As the monovalent organic group having 1 to 10 carbon atoms represented by R⁴⁸ and R⁴⁹, and the divalent alicyclic group or heterocyclic group having 3 to 10 ring members composed of R⁴⁸ and R⁴⁹ combined with each other together with a carbon atom to which R⁴⁸ and R⁴⁹ are bonded, a monovalent organic group having 1 to 10 carbon atoms represented by R⁴² and R⁴³ in the formula (2) and a divalent alicyclic group or heterocyclic group having 3 to 10 ring members composed of R⁴² and R⁴³ combined with each other together with a carbon atom to which R⁴² and R⁴³ are bonded can be suitably employed.

When R⁴⁷, R⁴⁸, or R⁴⁹ has a double bond other than an aromatic ring, two carbon atoms composing the double bond are bonded to 0, 1, or 3 hydrogen atoms in total.

Examples of Compound 2 represented by the formula (1-2-1) and formula (1-2-2) include the following compounds (m2-1) to (m2-11).

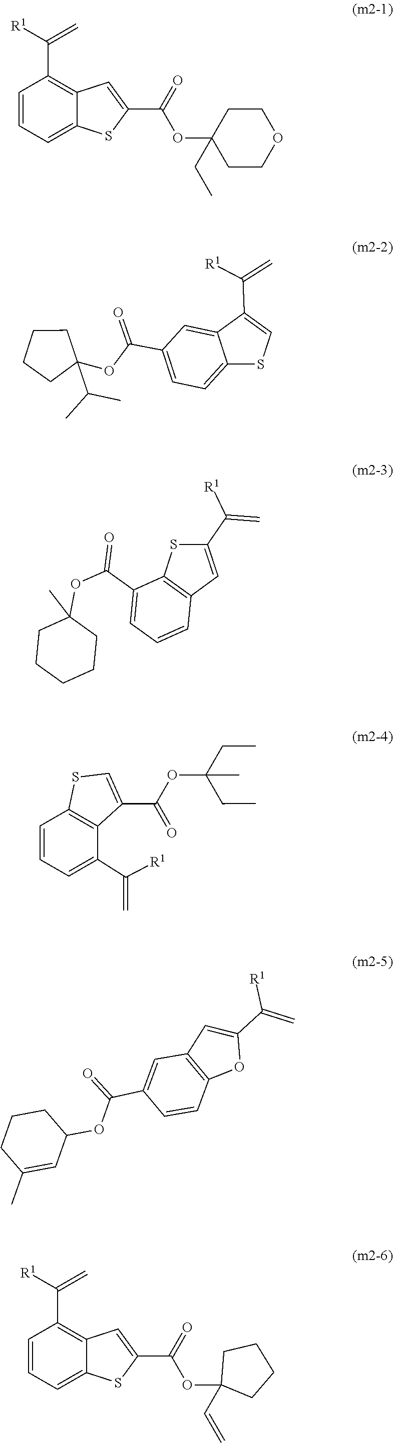

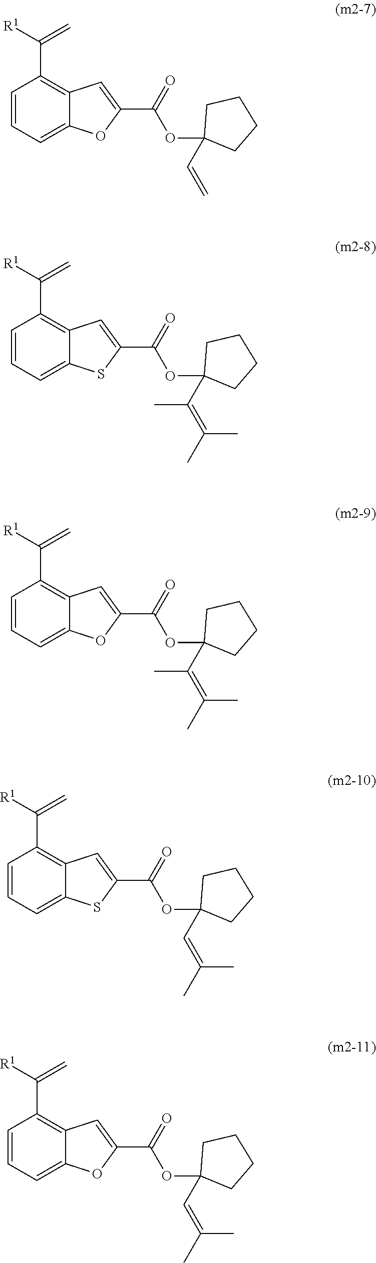

In the formula, R¹ has the same meanings as in the formulas (1-2-1) and (1-2-2).

### <Compound 3>

Compound 3 is represented by the following formula (1-3).

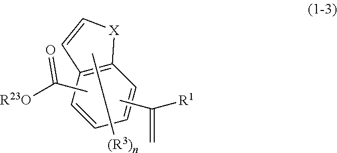

wherein in the formula (1-3),
X represents —S— or —O—;
R¹ is a hydrogen atom, a fluorine atom, a methyl group, or a trifluoromethyl group;
R²³ is a monovalent organic group having 3 or more carbon atoms;
R³ is a halogen atom, a hydroxy group, a cyano group, a nitro group, an amino group, a thiol group, or a monovalent organic group having 1 to 20 carbon atoms;
n is an integer of 0 to 4; and when n is 2 or more, a plurality of R³s are identical to or different from each other.
R¹, R³, and n have the same meaning as R¹, R³, and n in the formula (1).

As the monovalent organic group having 3 or more carbon atoms represented by R²³, a monovalent organic group having 3 or more carbon atoms among monovalent organic groups having 1 to 10 carbon atoms in the formula (2) can be suitably employed.

Examples of Compound 3 represented by the formula (1-3) include the following compounds (m3-1) to (m3-7).

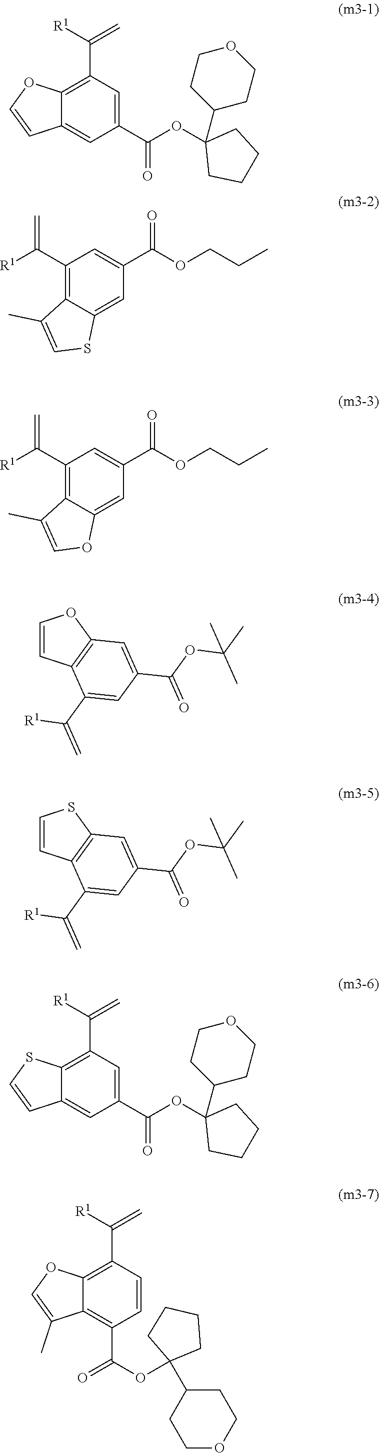

In the formula, R¹ has the same meanings as in the formula (1-3).

### <Compound 4>

Compound 4 is represented by the following formula (1-4).

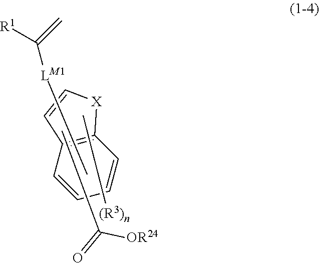

wherein in the formula (1-4),
X represents —S— or —O—;
R¹ is a hydrogen atom, a fluorine atom, a methyl group, or a trifluoromethyl group;
L^{M1} is a divalent linking group;
R²⁴ is a group represented by a following formula (2-3):

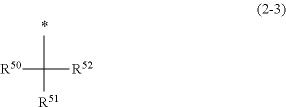

wherein in the formula (2-3),
R⁵⁰ is a hydrogen atom or a monovalent organic group having 1 to 10 carbon atoms;
R⁵¹ and R⁵² each independently represent a monovalent organic group having 1 to 10 carbon atoms, or a divalent alicyclic group or heterocyclic group having 3 to 10 ring members composed of R⁵¹ and R⁵² combined with each other together with a carbon atom to which R⁵¹ and R⁵² are bonded; and
* represents a bond bonded to an oxygen atom,
R³ is a halogen atom, a hydroxy group, a cyano group, a nitro group, an amino group, a thiol group, or a monovalent organic group having 1 to 20 carbon atoms;
n is an integer of 0 to 4; and when n is 2 or more, a plurality of R³s are identical to or different from each other.
R¹, R³, and n have the same meaning as R¹, R³, and n in the formula (1).

As the divalent linking group represented by L^{M1}, a divalent linking group represented by L¹ in the formula (1) can be suitably employed.

As the monovalent organic group having 1 to 10 carbon atoms represented by R⁵⁰, a monovalent organic group having 1 to 10 carbon atoms represented by R⁴¹ in the formula (2) can be suitably employed.

As the monovalent organic group having 1 to 10 carbon atoms represented by R⁵¹ and R⁵², and the divalent alicyclic group or heterocyclic group having 3 to 10 ring members composed of R⁵¹ and R⁵² combined with each other together with a carbon atom to which R⁵¹ and R⁵² are bonded, a monovalent organic group having 1 to 10 carbon atoms represented by R⁴² and R⁴³ in the formula (2) and a divalent alicyclic group or heterocyclic group having 3 to 10 ring members composed of R⁴² and R⁴³ combined with each other together with a carbon atom to which R⁴² and R⁴³ are bonded can be suitably employed.

Examples of Compound 4 represented by the formula (1-4) include the following compounds (m4-1) to (m4-13).

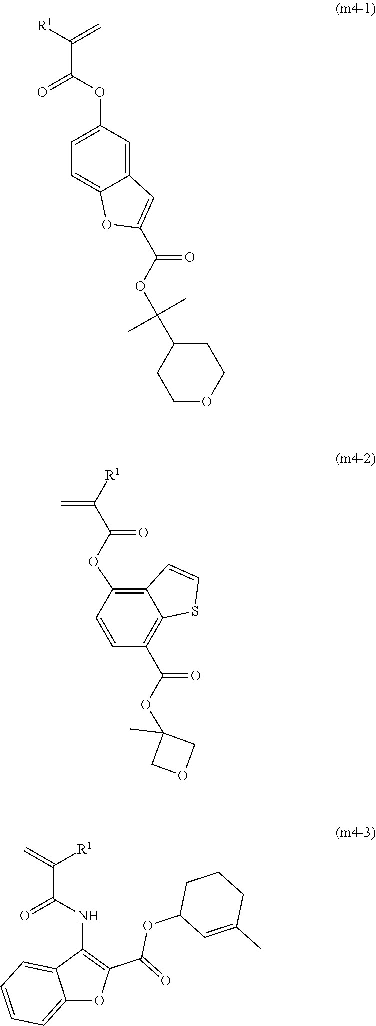

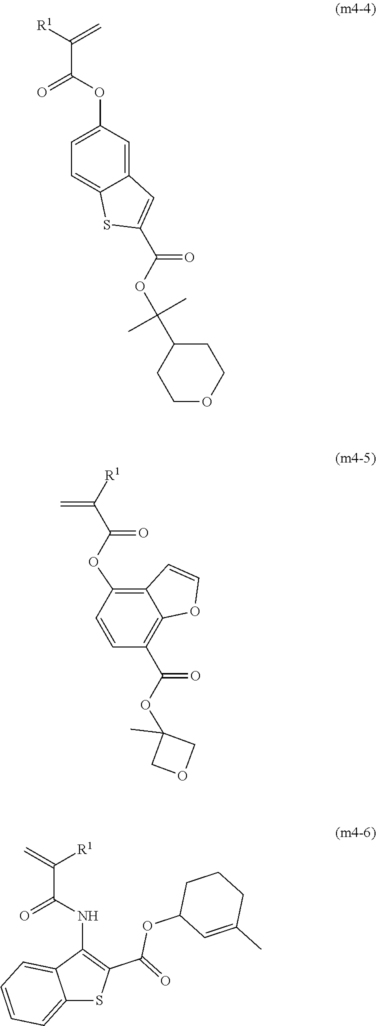

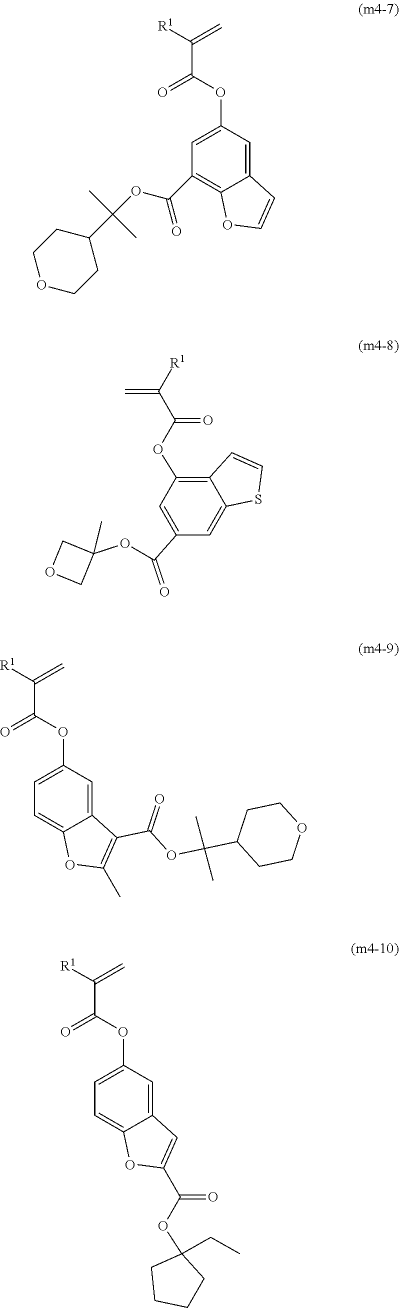

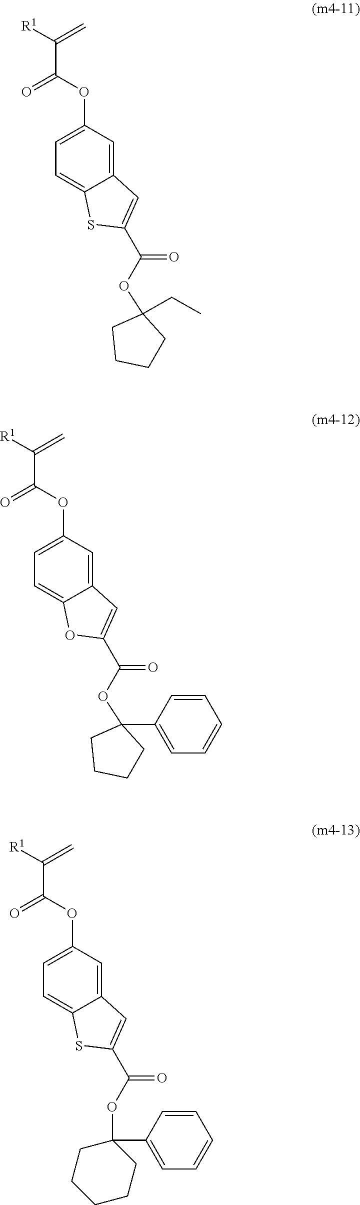

In the formula, R¹ has the same meanings as in the formula (1-4).

### <Compound 5>

Compound 5 is represented by the following formula (1-5).

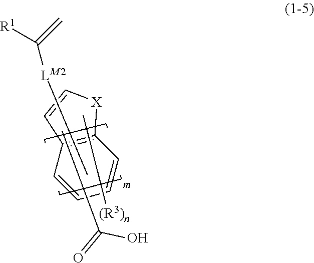

wherein in the formula (1-5),
X represents —S— or —O—;
R¹ is a hydrogen atom, a fluorine atom, a methyl group, or a trifluoromethyl group;
L^{M2} is a divalent linking group, provided that when m=0 and X is —O—, L^{M2} is a group represented by a following formula (3):

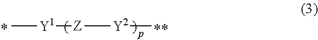

wherein in the formula (3),
Y¹ and Y² are each independently a single bond, —O—, —CO—, —CO—O—, —O—CO—, —O—CO—O—, —CO—NH—, —NH—CO—, or —S—, provided that when p is 0, Y¹ is —O—, —CO—, —CO—O—, —O—CO—, —O—CO—O—, —CO—NH—, —NH—CO—, or —S—;
p is an integer of 0 to 2;
Z is a divalent hydrocarbon group having 1 to 10 carbon atoms;
   and
* represents a bond bonded to a carbon atom composing a vinyl group, and ** represents a bond bonded to a carbon atom composing a heterocyclic ring,
R³ is a halogen atom, a hydroxy group, a cyano group, a nitro group, an amino group, a thiol group, or a monovalent organic group having 1 to 20 carbon atoms;
m is 0 or 1;
n is an integer of 0 to 4; and when n is 2 or more, a plurality of R³s are identical to or different from each other.
R¹, R³, and n have the same meaning as R¹, R³, and n in the formula (1).

As the divalent linking group represented by L^{M2}, a divalent linking group represented by L¹ in the formula (1) can be suitably employed, provided that when m=0 and X is —O—, L^{M2} is a group represented by the formula (3).

As the divalent hydrocarbon group having 1 to 10 carbon atoms represented by Z, a group obtained by removing one hydrogen atom from the monovalent hydrocarbon group having 1 to 20 carbon atoms in R² in the formula (1) can be suitably employed.

Examples of Compound 5 represented by the formula (1-5) include the following compounds (m5-1) to (m5-12).

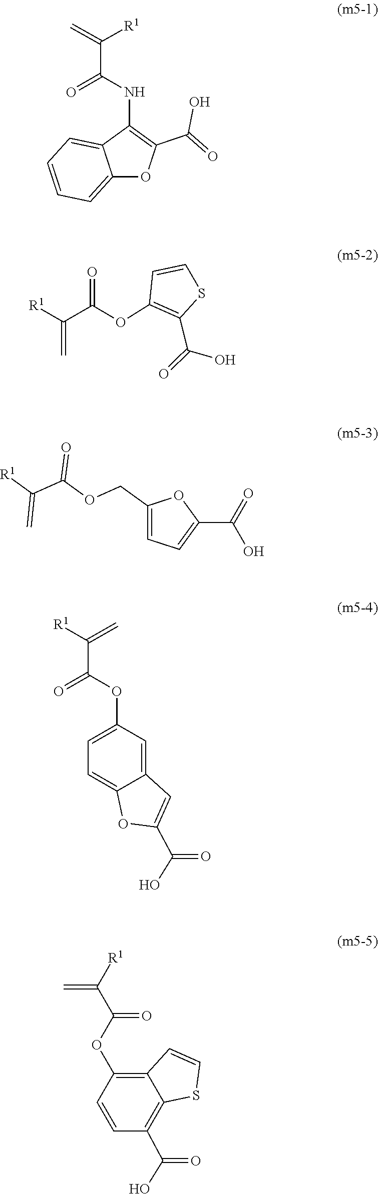

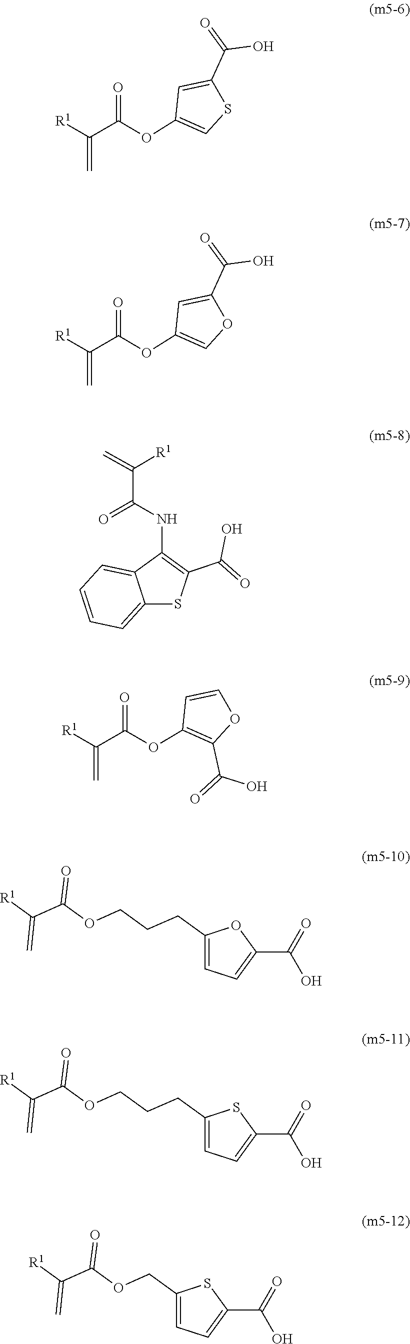

In the formula, R¹ has the same meanings as in the formula (1-5).

When the radiation-sensitive composition contains the polymer (A) containing the compound as a constituent monomer, a resist film having excellent sensitivity and CDU and suppressed occurrence of development defects can be formed.

### EXAMPLES

Hereinafter, the present invention will specifically be described with reference to Examples, but the present invention is not limited to these Examples. Various physical property values in Examples and Comparative Examples were measured by the following measurement methods.

### [Weight-Average Molecular Weight (Mw) and Number-Average Molecular Weight (Mn)]

Measurement was performed by gel permeation chromatography (GPC) with monodisperse polystyrene standards using GPC columns (G2000HXL×2, G3000HXL×1, and G4000HXL×1) manufactured by Tosoh Corporation, under analysis conditions including a flow rate: 1.0 mL/min, an elution solvent: tetrahydrofuran, a sample concentration: 1.0% by mass, an amount of sample injected: 100 μL, a column temperature: 40° C., and a detector: a differential refractometer. The degree of dispersion (Mw/Mn) was calculated from the measurement results of Mw and Mn.

[¹³C-NMR analysis]

¹³C-NMR analysis of polymers was performed using a nuclear magnetic resonance apparatus (“JNM-Delta400” manufactured by JEOL Ltd.).

### Synthesis of Compound (Monomer) [M]

### [Synthesis Example 1] (Synthesis of Compound (M-1))

A compound (M-1) was synthesized according to the following synthesis scheme.

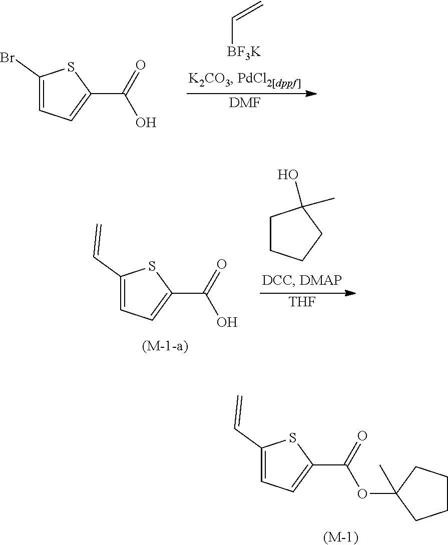

To a reaction vessel were added 145.0 mmol of 5-bromo-2-thiophenecarboxylic acid, 159.0 mmol of potassium vinyl trifluoroborate, 290.0 mmol of potassium carbonate, 2.90 mmol of [1,1′-bis(diphenylphosphino) ferrocene]dichloropalladium (II) and 300 g of dimethylformamide (DMF), followed by stirring at 100° C. for 10 hours. The reaction solution was cooled to 20° C. or lower, a saturated aqueous ammonium chloride solution was added to terminate the reaction, and then extraction was performed using ethyl acetate. The resulting organic layer was washed with water and dried over sodium sulfate, the solvent was then distilled off, and purification was performed by column chromatography, affording a compound represented by the formula (M-1-a) in a good yield.

To the compound represented by the formula (M-1-a) were added 195.0 mmol of 1-methylcyclopentanol, 143.0 mmol of N,N′-dicyclohexylcarbodiimide (DCC), 13.0 mmol of 4-dimethylaminopyridine (DMAP) and 250 g of tetrahydrofuran (THF), followed by stirring at room temperature for 5 hours. The reaction solution was then filtered, followed by addition of a saturated aqueous ammonium chloride solution. Thereafter, ethyl acetate was added to perform extraction. The resulting organic layer was washed with water and dried over sodium sulfate. Thereafter, the solvent was distilled off, and purification was performed by column chromatography, affording a compound represented by the formula (M-1) in a good yield.

### [Synthesis Examples 2 to 9] (Synthesis of Monomer (M-2) to Monomer (M-9))

Compounds represented by the following formulas (M-2) to (M-9) were synthesized in the same manner as in Synthesis Example 1 except that the raw materials and the precursor were appropriately changed. Hereinafter, the compounds represented by the formulas (M-2) to (M-9) may be respectively referred to as “compound (M-2)” to “compound (M-9)” or “monomer (M-2)” to “monomer (M-9)”.

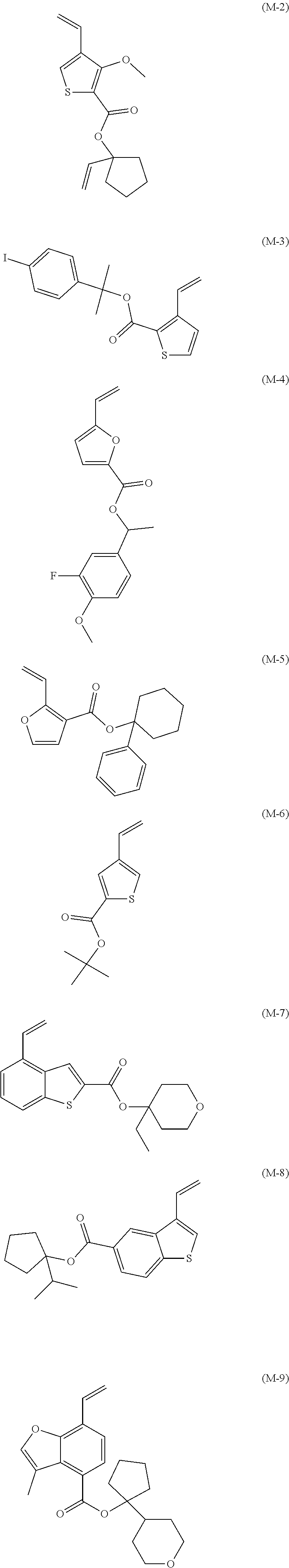

### [Synthesis Example 10] (Synthesis of Compound (M-10))

A compound (M-10) was synthesized according to the following synthesis scheme.

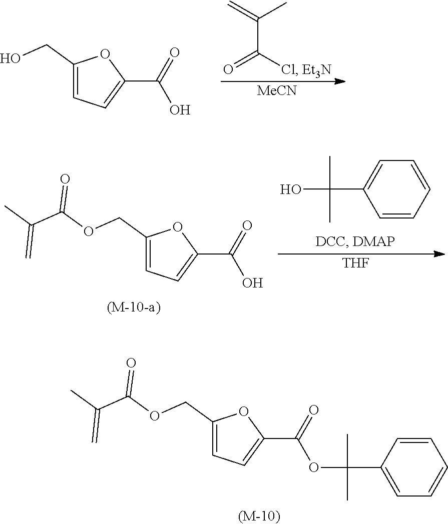

To a reaction vessel were added 141.0 mmol of 5-(hydroxymethyl) furan-2-carboxylic acid, 169.0 mmol of triethylamine and 200 g of acetonitrile, followed by stirring at 0° C. for 1 hour. Thereafter, 169.0 mmol of methacrylic acid chloride was slowly added dropwise, followed by stirring at 60° C. for 2 hours. The reaction solution was cooled to 20° C. or lower, a saturated aqueous ammonium chloride solution was added to terminate the reaction, and then extraction was performed using ethyl acetate. The resulting organic layer was washed with water and dried over sodium sulfate. Thereafter, the solvent was distilled off, and purification was performed by column chromatography, affording a compound represented by the formula (M-10-a) in a good yield.

To the compound represented by the formula (M-10-a) were added 143.0 mmol of 2-phenyl-2-propanol, 105.0 mmol of N,N′-dicyclohexylcarbodiimide, 9.5 mmol of 4-dimethylaminopyridine, and 250 g of tetrahydrofuran, followed by stirring at room temperature for 5 hours. The reaction solution was then filtered, followed by addition of a saturated aqueous ammonium chloride solution. Thereafter, ethyl acetate was added to perform extraction. The resulting organic layer was washed with water and dried over sodium sulfate. Thereafter, the solvent was distilled off, and purification was performed by column chromatography, affording a compound represented by the formula (M-10) in a good yield.

### [Synthesis Examples 11 to 14] (Synthesis of Monomer (M-11) to Monomer (M-14))

Compounds represented by the following formulas (M-11) to (M-14) were synthesized in the same manner as in Synthesis Example 10 except that the precursor was appropriately changed. Hereinafter, the compounds represented by the formulas (M-11) to (M-14) may be respectively referred to as “compound (M-11)” to “compound (M-14)” or “monomer (M-11)” to “monomer (M-14)”.

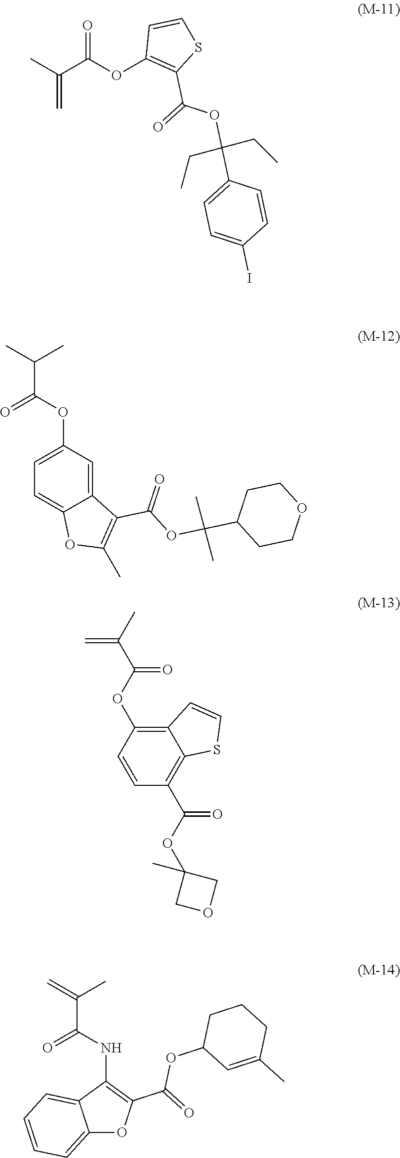

Among the monomers used in the synthesis of the polymers in each Example and each Comparative Example, the structures of monomers other than the monomer having the partial structure represented by the formula (1) are shown.

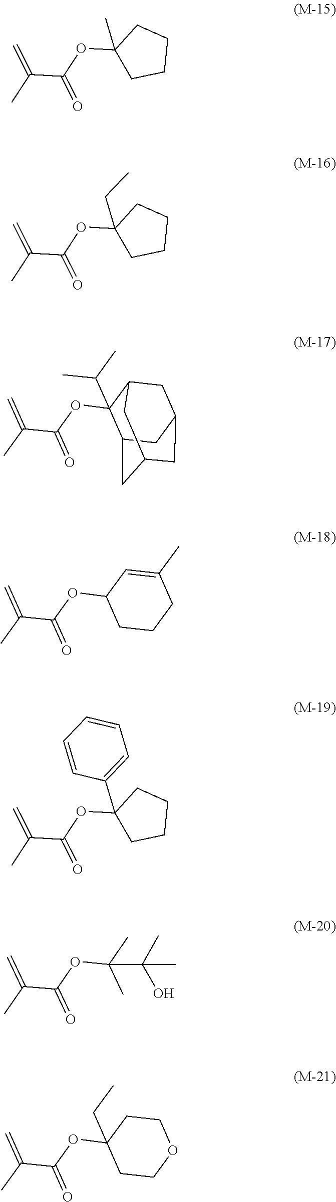

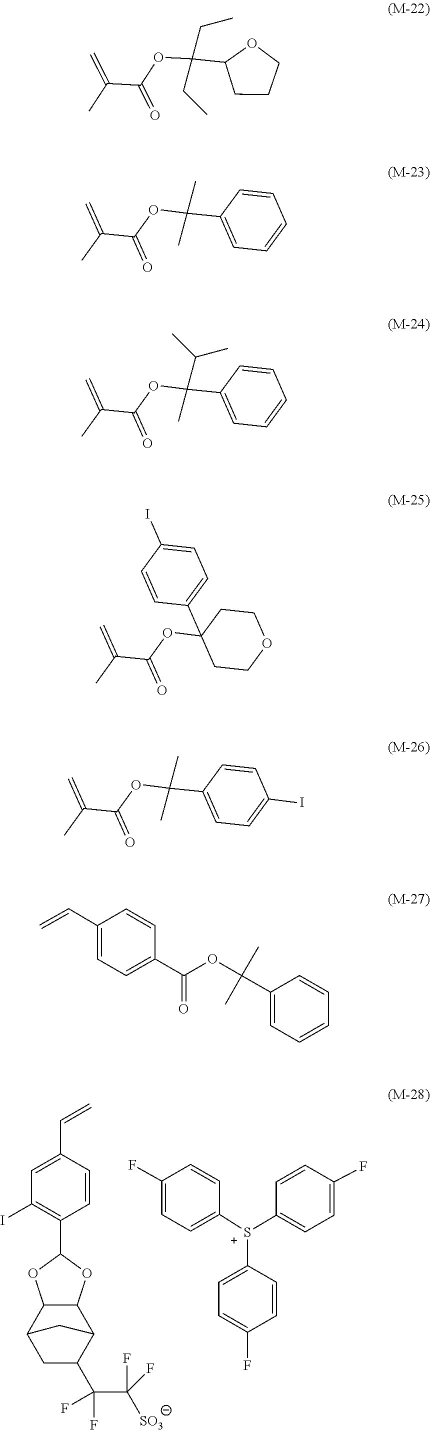

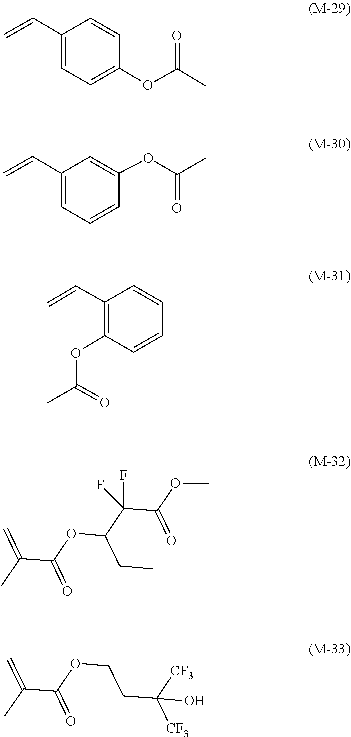

### [Polymer Synthesis Example 1] Synthesis of Polymer (P-1)

A compound (M-1), a compound (M-15), and a compound (M-29) as monomers were dissolved in 1-methoxy-2-propanol (200 parts by mass with respect to the total amount of the monomers) to have a molar ratio of 20/40/40. Next, 2,2′-azobis(methyl isobutyrate) was added as an initiator in an amount of 10 mol % with respect to all the monomers to prepare a monomer solution. Meanwhile, to an empty reaction vessel was added 1-methoxy-2-propanol (100 parts by mass with respect to the total amount of monomers), followed by heating to 85° C. with stirring. Next, the monomer solution prepared above was added dropwise over 3 hours, followed by further heating at 85° C. for 3 hours to perform a polymerization reaction for 6 hours in total. After completion of the polymerization reaction, the polymerization solution was cooled to room temperature.

The cooled polymerization solution was charged into hexane (500 parts by mass with respect to the polymerization solution), and a precipitated white powder was separated by filtration. The white powder separated by filtration was washed twice with 100 parts by mass of hexane with respect to the polymerization solution, then separated by filtration, and dissolved in 1-methoxy-2-propanol (300 parts by mass). Next, methanol (300 parts by mass), triethylamine (50 parts by mass), and ultrapure water (10 parts by mass) were added, and a hydrolysis reaction was performed at 70° C. for 6 hours with stirring.

After completion of the reaction, the remaining solvent was distilled off, and the resulting solid was dissolved in acetone (100 parts by mass). The resulting solution was added dropwise into 500 parts by mass of water to permit coagulation of the resin. The resulting solid was separated by filtration. The resulting solid was dried at 50° C. for 12 hours to yield a white powdery polymer (P-1). The resulting polymer (P-1) had an Mw of 5,000 and an Mw/Mn of 1.6. As a result of 13C-NMR analysis, the content of a structural unit derived from the compound (M-1): a structural unit derived from the compound (M-15): a structural unit derived from the compound (M-29) was 18:39: 43 (mol %).

### [Polymer Synthesis Examples 2 to 32] (Synthesis of Polymers (P-2) to (P-32))

Polymers (P-2) to (P-32) in which prescribed amounts of monomers of the types shown in Table 1 were blended were obtained in the same manner as in Polymer Synthesis Example 1. Mw and Mw/Mn of the respective polymers obtained are shown together in Table 1. For example, “M-29/M-30”= “20/20” in Synthesis Example 28 means that M-29 and M-30 are each contained in an amount of 20 mol %, and the total amount is 40 mol %. Accordingly, the composition of the base polymer (P-28) is M-1/M-15/M-29/M-30=20/40/20/20 (mol %).

TABLE 1

| | | Blending amount of compound (mol %) | | | | | | | | | |
|---|---|---|---|---|---|---|---|---|---|---|---|
| | | Structural unit | | Structural unit | | Structural unit | | Structural unit | | | |
| | | [I] | | [III] | | [II] | | [IV] | | Physical | |
| | | | Blending | | Blending | | Blending | | Blending | property value | |
| | Polymer | Type | amount | Type | amount | Type | amount | Type | amount | Mw | Mw/Mn |
| Synthesis | P-1 | M-1 | 20 | M-15 | 40 | M-29 | 40 | — | — | 5000 | 1.6 |
| Example 1 | | | | | | | | | | | |
| Synthesis | P-2 | M-2 | 20 | M-15 | 40 | M-29 | 40 | — | — | 5500 | 1.5 |
| Example 2 | | | | | | | | | | | |
| Synthesis | P-3 | M-3 | 20 | M-15 | 40 | M-29 | 40 | — | — | 5300 | 1.5 |
| Example 3 | | | | | | | | | | | |
| Synthesis | P-4 | M-4 | 20 | M-15 | 40 | M-29 | 40 | — | — | 4900 | 1.6 |
| Example 4 | | | | | | | | | | | |
| Synthesis | P-5 | M-5 | 20 | M-15 | 40 | M-29 | 40 | — | — | 5200 | 1.4 |
| Example 5 | | | | | | | | | | | |
| Synthesis | P-6 | M-6 | 20 | M-15 | 40 | M-29 | 40 | — | — | 5500 | 1.5 |
| Example 6 | | | | | | | | | | | |
| Synthesis | P-7 | M-7 | 20 | M-15 | 40 | M-29 | 40 | — | — | 5000 | 1.6 |
| Example 7 | | | | | | | | | | | |
| Synthesis | P-8 | M-8 | 20 | M-15 | 40 | M-29 | 40 | — | — | 5400 | 1.6 |
| Example 8 | | | | | | | | | | | |
| Synthesis | P-9 | M-9 | 20 | M-15 | 40 | M-29 | 40 | — | — | 5300 | 1.4 |
| Example 9 | | | | | | | | | | | |
| Synthesis | P-10 | M-10 | 20 | M-15 | 40 | M-29 | 40 | — | — | 5500 | 1.6 |
| Example 10 | | | | | | | | | | | |
| Synthesis | P-11 | M-11 | 20 | M-15 | 40 | M-29 | 40 | — | — | 5700 | 1.5 |
| Example 11 | | | | | | | | | | | |
| Synthesis | P-12 | M-12 | 20 | M-15 | 40 | M-29 | 40 | — | — | 5200 | 1.4 |
| Example 12 | | | | | | | | | | | |
| Synthesis | P-13 | M-13 | 20 | M-15 | 40 | M-29 | 40 | — | — | 5300 | 1.6 |
| Example 13 | | | | | | | | | | | |
| Synthesis | P-14 | M-14 | 20 | M-15 | 40 | M-29 | 40 | — | — | 5300 | 1.6 |
| Example 14 | | | | | | | | | | | |
| Synthesis | P-15 | M-1 | 20 | M-16 | 40 | M-29 | 40 | — | — | 5100 | 1.5 |
| Example 15 | | | | | | | | | | | |
| Synthesis | P-16 | M-1 | 20 | M-17 | 40 | M-29 | 40 | — | — | 5200 | 1.5 |
| Example 16 | | | | | | | | | | | |
| Synthesis | P-17 | M-1 | 20 | M-18 | 40 | M-29 | 40 | — | — | 4900 | 1.4 |
| Example 17 | | | | | | | | | | | |
| Synthesis | P-18 | M-1 | 20 | M-19 | 40 | M-29 | 40 | — | — | 4900 | 1.5 |
| Example 18 | | | | | | | | | | | |
| Synthesis | P-19 | M-1 | 20 | M-20 | 40 | M-29 | 40 | — | — | 4800 | 1.5 |
| Example 19 | | | | | | | | | | | |
| Synthesis | P-20 | M-1 | 20 | M-21 | 40 | M-29 | 40 | — | — | 5100 | 1.6 |
| Example 20 | | | | | | | | | | | |
| Synthesis | P-21 | M-1 | 20 | M-22 | 40 | M-29 | 40 | — | — | 5000 | 1.5 |
| Example 21 | | | | | | | | | | | |
| Synthesis | P-22 | M-1 | 20 | M-23 | 40 | M-29 | 40 | — | — | 5000 | 1.4 |
| Example 22 | | | | | | | | | | | |
| Synthesis | P-23 | M-1 | 20 | M-24 | 40 | M-29 | 40 | — | — | 4700 | 1.5 |
| Example 23 | | | | | | | | | | | |
| Synthesis | P-24 | M-1 | 20 | M-25 | 40 | M-29 | 40 | — | — | 4900 | 1.5 |
| Example 24 | | | | | | | | | | | |
| Synthesis | P-25 | M-1 | 20 | M-26 | 40 | M-29 | 40 | — | — | 5100 | 1.5 |
| Example 25 | | | | | | | | | | | |
| Synthesis | P-26 | M-1 | 20 | M-27 | 40 | M-29 | 40 | — | — | 5500 | 1.6 |
| Example 26 | | | | | | | | | | | |
| Synthesis | P-27 | M-1 | 20 | M-15 | 20 | M-29 | 40 | M-28 | 20 | 5100 | 1.4 |
| Example 27 | | | | | | | | | | | |
| Synthesis | P-28 | M-1 | 20 | M-15 | 40 | M-29/ | 20/20 | — | — | 5300 | 1.6 |
| Example 28 | | | | | | M-30 | | | | | |
| Synthesis | P-29 | M-1 | 20 | M-15 | 40 | M-29/ | 20/20 | — | — | 5800 | 1.5 |
| Example 29 | | | | | | M-31 | | | | | |
| Synthesis | P-30 | M-1 | 30 | M-15 | 30 | M-29 | 40 | — | — | 6200 | 1.4 |
| Example 30 | | | | | | | | | | | |
| Synthesis | P-31 | M-1 | 10 | M-15 | 50 | M-29 | 40 | — | — | 6600 | 1.6 |
| Example 31 | | | | | | | | | | | |
| Synthesis | P-32 | — | — | M-15/ | 40/40 | M-29 | 20 | — | — | 6000 | 1.6 |
| Example 32 | | | | M-27 | | | | | | | |

### Synthesis of High Fluorine-Content Polymer

### [Synthesis Example 33] (Synthesis of Polymer (F-1))

Compounds (M-16) and (M-32) as monomers were dissolved in 2-butanone (200 parts by mass) to have a molar ratio of 30/70. To this was added azobisisobutyronitrile (AIBN) (5 mol % with respect to the total amount of monomers) as an initiator to prepare a monomer solution. In a reaction vessel was placed 2-butanone (100 parts by mass), followed by purge with nitrogen for 30 minutes. The temperature in the reaction vessel was adjusted to 80° C., and the monomer solution was added dropwise to the reaction vessel over 3 hours with stirring. A polymerization reaction was performed for 6 hours with the start of dropwise addition as the start time of the polymerization reaction. After completion of the polymerization reaction, the polymerization solution was cooled with water to 30° C. or lower. The solvent was replaced with acetonitrile (400 parts by mass). Hexane (100 parts by mass) was then added, followed by stirring, whereby an acetonitrile layer was collected. The operation was repeated three times. By replacing the solvent with propylene glycol monomethyl ether acetate, a solution of polymer (F-1) was obtained in a good yield.

### [Synthesis Example 34] (Synthesis of Polymer (F-2))

A polymer (F-2) in which prescribed amounts of monomers of the types shown in Table 2 were blended was obtained in the same manner as in Synthesis Example 33. Mw and Mw/Mn of the respective polymers obtained are shown together in Table 2.

TABLE 2

| | High fluorine- | Blending amount of compound (mol %) | | | | Physical | |
|---|---|---|---|---|---|---|---|
| | content polymer | | Blending | | Blending | property value | |
| | (F) | Type | amount | Type | amount | Mw | Mw/Mn |
| Synthesis | F-1 | M-16 | 30 | M-32 | 70 | 5400 | 1.7 |
| Example 33 | | | | | | | |
| Synthesis | F-2 | M-17 | 30 | M-33 | 70 | 5600 | 1.7 |
| Example 34 | | | | | | | |

### <Preparation of Radiation-Sensitive Composition>

The radiation-sensitive acid generator (C), the acid diffusion controlling agent (D), and the solvent (B) composing the radiation-sensitive composition are described below.

### [Radiation-Sensitive Acid Generator (C)]

Compounds represented by the following formulas (C-1) to (C-8)

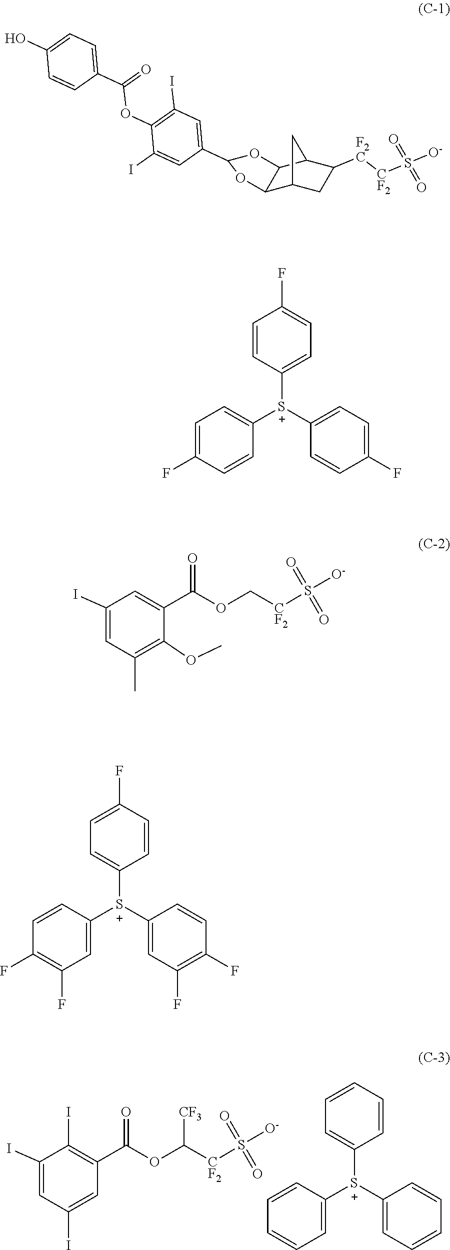 [SMILES: O=C(Oc1c(I)cc(C2OC3C4CC(C3O2)C(C(F)(F)C(F)(F)S(=O)(=O)[O-])C4)cc1I)c1ccc(O)cc1 ; Fc1ccc([S+](c2ccc(F)cc2)c2ccc(F)cc2)cc1 ; COc1c(C)cc(I)cc1C(=O)OCC(F)(F)S(=O)(=O)[O-] ; Fc1ccc([S+](c2ccc(F)c(F)c2)c2ccc(F)c(F)c2)cc1 ; O=C(OC(C(F)(F)F)C(F)(F)S(=O)(=O)[O-])c1cc(I)cc(I)c1I ; c1ccc([S+](c2ccccc2)c2ccccc2)cc1]

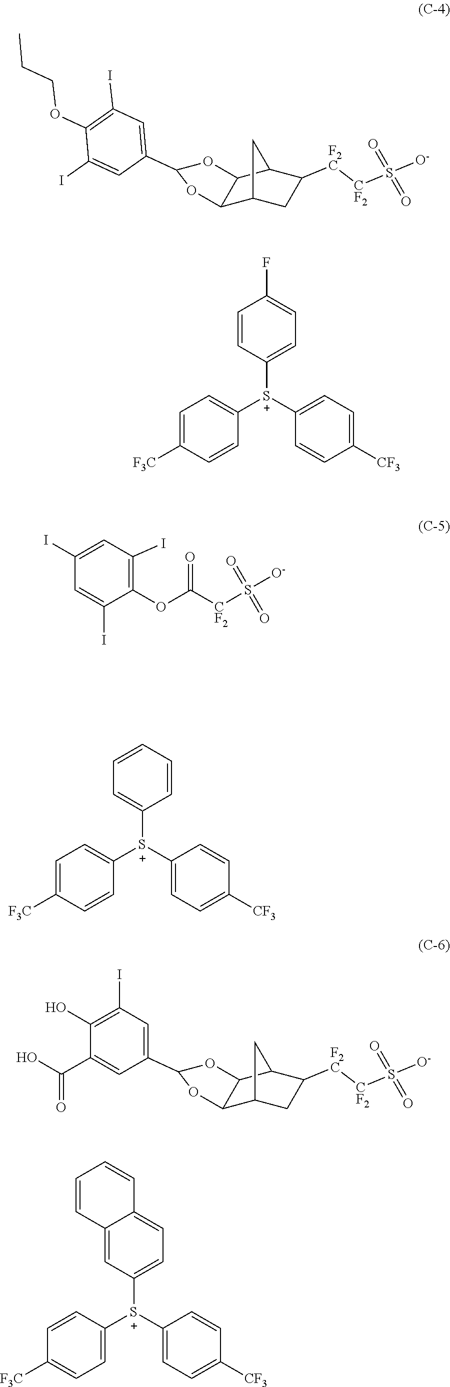

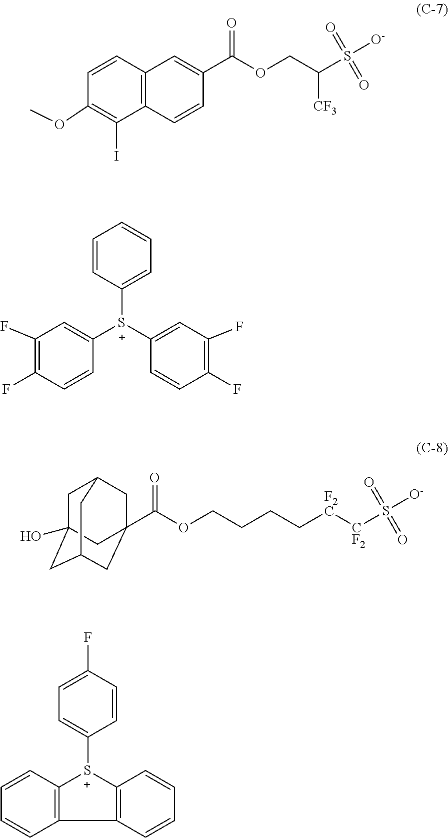 [SMILES: COc1ccc2cc(C(=O)OCC(C(F)(F)F)S(=O)(=O)[O-])ccc2c1I ; Fc1ccc([S+](c2ccccc2)c2ccc(F)c(F)c2)cc1F ; O=C(OCCCCC(F)(F)C(F)(F)S(=O)(=O)[O-])C12CC3CC(CC(O)(C3)C1)C2 ; Fc1ccc(-[s+]2c3ccccc3c3ccccc32)cc1]

### [Acid Diffusion Controlling Agent (D)]

Compounds represented by the following formulas (D-1) to (D-6)

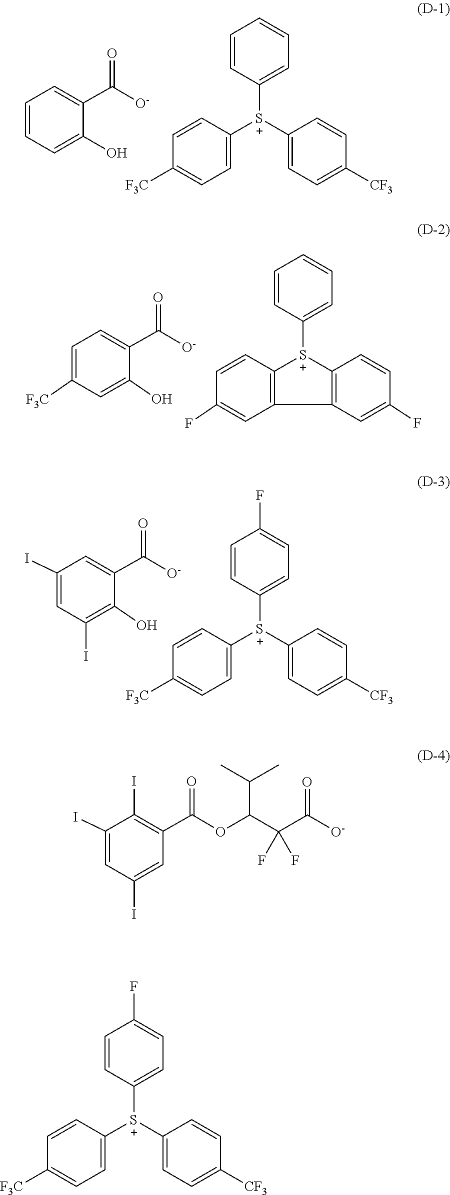

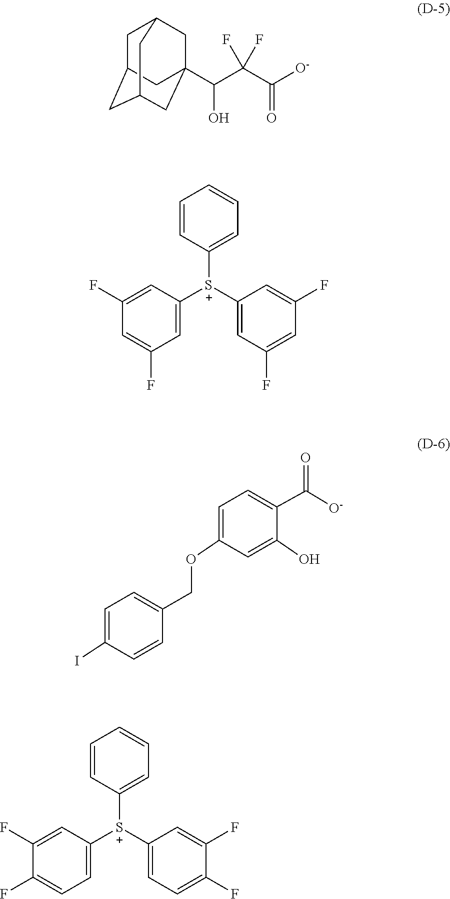 [SMILES: O=C([O-])C(F)(F)C(O)C12CC3CC(CC(C3)C1)C2 ; Fc1cc(F)cc([S+](c2ccccc2)c2cc(F)cc(F)c2)c1 ; O=C([O-])c1ccc(OCc2ccc(I)cc2)cc1O ; Fc1ccc([S+](c2ccccc2)c2ccc(F)c(F)c2)cc1F]

### [Organic Solvent (B)]

B-1: Propylene glycol monomethyl ether acetate
B-2: Propylene glycol monomethyl ether

### [Preparation of Radiation-Sensitive Composition]

### Example 1

Mixed were 100 parts by mass of (P-1) as a polymer, 45 parts by mass of (C-1) as a radiation-sensitive acid generator, 50 mol % of (D-1) as an acid diffusion controlling agent with respect to (C-1), 3 parts by mass of (F-1) as a high-fluorine-containing polymer, 5,500 parts by mass of (B-1) as an organic solvent, and 1500 parts by mass of (B-2). This mixture was filtered through a membrane filter having a pore size of 0.2 μm, whereby the radiation-sensitive composition (R-1) was prepared.

### Examples 2 to 46 and Comparative Examples 1 to 3

Radiation-sensitive compositions (R-2) to (R-46) and (CR-1) to (CR-3) were prepared in the same manner as in Example 1 except that the types and the blending amounts of respective components shown in the following Table were used, provided that in Example 27, 50 mol % of the acid diffusion controlling agent (D) was mixed with respect to the part by mass of the structural unit (IV) in the polymer (P-27), and in Example 28, 50 mol % of the acid diffusion controlling agent (D) was mixed with respect to the total part by mass of the structural unit (IV) in the polymer (P-27) and the radiation-sensitive acid generator (C).

TABLE 3

| | | | | Radiation- | | | | High fluorine- | | | |
|---|---|---|---|---|---|---|---|---|---|---|---|
| | | | | sensitive acid | | | | content | | | |
| | | Polymer (A) | | generator (C) | | Acid diffusion | | polymer (F) | | Solvent (B) | |
| | | | Content | | Content | controlling | | | Content | | Content |
| | Radiation- | | (Parts | | (Parts | agent (D) | | | (Parts | | (Parts |
| | sensitive | | by | | by | | Content | | by | | by |
| | composition | Type | mass) | Type | mass) | Type | (mol %) | Type | mass) | Type | mass) |
| Example 1 | R-1 | P-1 | 100 | C-1 | 45 | D-1 | 50 | F-1 | 3 | B-1/B-2 | 5,500/1,500 |
| Example 2 | R-2 | P-2 | 100 | C-1 | 45 | D-1 | 50 | F-1 | 3 | B-1/B-2 | 5,500/1,500 |
| Example 3 | R-3 | P-3 | 100 | C-1 | 45 | D-1 | 50 | F-1 | 3 | B-1/B-2 | 5,500/1,500 |
| Example 4 | R-4 | P-4 | 100 | C-1 | 45 | D-1 | 50 | F-1 | 3 | B-1/B-2 | 5,500/1,500 |
| Example 5 | R-5 | P-5 | 100 | C-1 | 45 | D-1 | 50 | F-1 | 3 | B-1/B-2 | 5,500/1,500 |
| Example 6 | R-6 | P-6 | 100 | C-1 | 45 | D-1 | 50 | F-1 | 3 | B-1/B-2 | 5,500/1,500 |
| Example 7 | R-7 | P-7 | 100 | C-1 | 45 | D-1 | 50 | F-1 | 3 | B-1/B-2 | 5,500/1,500 |
| Example 8 | R-8 | P-8 | 100 | C-1 | 45 | D-1 | 50 | F-1 | 3 | B-1/B-2 | 5,500/1,500 |
| Example 9 | R-9 | P-9 | 100 | C-1 | 45 | D-1 | 50 | F-1 | 3 | B-1/B-2 | 5,500/1,500 |
| Example 10 | R-10 | P-10 | 100 | C-1 | 45 | D-1 | 50 | F-1 | 3 | B-1/B-2 | 5,500/1,500 |
| Example 11 | R-11 | P-11 | 100 | C-1 | 45 | D-1 | 50 | F-1 | 3 | B-1/B-2 | 5,500/1,500 |
| Example 12 | R-12 | P-12 | 100 | C-1 | 45 | D-1 | 50 | F-1 | 3 | B-1/B-2 | 5,500/1,500 |
| Example 13 | R-13 | P-13 | 100 | C-1 | 45 | D-1 | 50 | F-1 | 3 | B-1/B-2 | 5,500/1,500 |
| Example 14 | R-14 | P-14 | 100 | C-1 | 45 | D-1 | 50 | F-1 | 3 | B-1/B-2 | 5,500/1,500 |
| Example 15 | R-15 | P-15 | 100 | C-1 | 45 | D-1 | 50 | F-1 | 3 | B-1/B-2 | 5,500/1,500 |
| Example 16 | R-16 | P-16 | 100 | C-1 | 45 | D-1 | 50 | F-1 | 3 | B-1/B-2 | 5,500/1,500 |
| Example 17 | R-17 | P-17 | 100 | C-1 | 45 | D-1 | 50 | F-1 | 3 | B-1/B-2 | 5,500/1,500 |
| Example 18 | R-18 | P-18 | 100 | C-1 | 45 | D-1 | 50 | F-1 | 3 | B-1/B-2 | 5,500/1,500 |
| Example 19 | R-19 | P-19 | 100 | C-1 | 45 | D-1 | 50 | F-1 | 3 | B-1/B-2 | 5,500/1,500 |
| Example 20 | R-20 | P-20 | 100 | C-1 | 45 | D-1 | 50 | F-1 | 3 | B-1/B-2 | 5,500/1,500 |
| Example 21 | R-21 | P-21 | 100 | C-1 | 45 | D-1 | 50 | F-1 | 3 | B-1/B-2 | 5,500/1,500 |
| Example 22 | R-22 | P-22 | 100 | C-1 | 45 | D-1 | 50 | F-1 | 3 | B-1/B-2 | 5,500/1,500 |
| Example 23 | R-23 | P-23 | 100 | C-1 | 45 | D-1 | 50 | F-1 | 3 | B-1/B-2 | 5,500/1,500 |
| Example 24 | R-24 | P-24 | 100 | C-1 | 45 | D-1 | 50 | F-1 | 3 | B-1/B-2 | 5,500/1,500 |
| Example 25 | R-25 | P-25 | 100 | C-1 | 45 | D-1 | 50 | F-1 | 3 | B-1/B-2 | 5,500/1,500 |
| Example 26 | R-26 | P-26 | 100 | C-1 | 45 | D-1 | 50 | F-1 | 3 | B-1/B-2 | 5,500/1,500 |
| Example 27 | R-27 | P-27 | 100 | — | — | D-1 | 50 | F-1 | 3 | B-1/B-2 | 5,500/1,500 |
| Example 28 | R-28 | P-27 | 100 | C-1 | 25 | D-1 | 50 | F-1 | 3 | B-1/B-2 | 5,500/1,500 |
| Example 29 | R-29 | P-28 | 100 | C-1 | 45 | D-1 | 50 | F-1 | 3 | B-1/B-2 | 5,500/1,500 |
| Example 30 | R-30 | P-29 | 100 | C-1 | 45 | D-1 | 50 | F-1 | 3 | B-1/B-2 | 5,500/1,500 |
| Example 31 | R-31 | P-30 | 100 | C-1 | 45 | D-1 | 50 | F-1 | 3 | B-1/B-2 | 5,500/1,500 |
| Example 32 | R-32 | P-31 | 100 | C-1 | 45 | D-1 | 50 | F-1 | 3 | B-1/B-2 | 5,500/1,500 |
| Example 33 | R-33 | P-1 | 100 | C-2 | 45 | D-1 | 50 | F-1 | 3 | B-1/B-2 | 5,500/1,500 |
| Example 34 | R-34 | P-1 | 100 | C-3 | 45 | D-1 | 50 | F-1 | 3 | B-1/B-2 | 5,500/1,500 |
| Example 35 | R-35 | P-1 | 100 | C-4 | 45 | D-1 | 50 | F-1 | 3 | B-1/B-2 | 5,500/1,500 |
| Example 36 | R-36 | P-1 | 100 | C-5 | 45 | D-1 | 50 | F-1 | 3 | B-1/B-2 | 5,500/1,500 |
| Example 37 | R-37 | P-1 | 100 | C-6 | 45 | D-1 | 50 | F-1 | 3 | B-1/B-2 | 5,500/1,500 |
| Example 38 | R-38 | P-1 | 100 | C-7 | 45 | D-1 | 50 | F-1 | 3 | B-1/B-2 | 5,500/1,500 |
| Example 39 | R-39 | P-3 | 100 | C-8 | 45 | D-1 | 50 | F-1 | 3 | B-1/B-2 | 5,500/1,500 |
| Example 40 | R-40 | P-1 | 100 | C-1 | 45 | D-2 | 50 | F-1 | 3 | B-1/B-2 | 5,500/1,500 |
| Example 41 | R-41 | P-1 | 100 | C-1 | 45 | D-3 | 50 | F-1 | 3 | B-1/B-2 | 5,500/1,500 |
| Example 42 | R-42 | P-1 | 100 | C-1 | 45 | D-4 | 50 | F-1 | 3 | B-1/B-2 | 5,500/1,500 |
| Example 43 | R-43 | P-1 | 100 | C-1 | 45 | D-5 | 50 | F-1 | 3 | B-1/B-2 | 5,500/1,500 |
| Example 44 | R-44 | P-1 | 100 | C-1 | 45 | D-6 | 50 | F-1 | 3 | B-1/B-2 | 5,500/1,500 |
| Example 45 | R-45 | P-1 | 100 | C-1 | 45 | D-1 | 50 | F-2 | 3 | B-1/B-2 | 5,500/1,500 |
| Example 46 | R-46 | P-1 | 100 | C-1 | 45 | D-1 | 50 | — | — | B-1/B-2 | 5,500/1,500 |
| Comparative | CR-1 | P-1 | 100 | C-8 | 45 | D-1 | 50 | F-1 | 3 | B-1/B-2 | 5,500/1,500 |
| Example 1 | | | | | | | | | | | |
| Comparative | CR-2 | P-32 | 100 | C-1 | 45 | D-1 | 50 | F-1 | 3 | B-1/B-2 | 5,500/1,500 |
| Example 2 | | | | | | | | | | | |
| Comparative | CR-3 | P-32 | 100 | C-8 | 45 | D-1 | 50 | F-1 | 3 | B-1/B-2 | 5,500/1,500 |
| Example 3 | | | | | | | | | | | |

### <Formation of Resist Pattern>

Each of the radiation-sensitive compositions prepared as described above was applied using a spin coater (CLEAN TRACK ACT12, manufactured by Tokyo Electron Ltd.) to a surface of a 12-inch silicon wafer with a 20 nm thick underlayer film (AL412, manufactured by Brewer Science). Prebaking (PB) was performed at 100° C. for 60 seconds, followed by cooling at 23° C. for 30 seconds, whereby a resist film having a thickness of 30 nm was formed. The resist film was irradiated with EUV light using an EUV exposure machine (model “NXE3300”, manufactured by ASML, NA=0.33, lighting condition: Conventional, s=0.89). Then, the resist film was subjected to post exposure baking (PEB) at 100° C. for 60 seconds. Subsequently, development was performed at 23° C. for 30 seconds using a 2.38% by mass aqueous TMAH solution to form a positive-tone 50 nm-pitch 25 nm contact hole pattern.

### <Evaluation>

The sensitivity, the CDU, and the number of development defects of each of the radiation-sensitive compositions were evaluated by measuring each of the formed resist patterns in accordance with the following methods. Note that a scanning electron microscope (“CG-5000” manufactured by Hitachi High-Tech Corporation) was used for measurement of the resist pattern. The evaluation results are shown in Table 4.

### [Sensitivity]

The exposure amount at which a 25 nm contact hole pattern was formed in the formation of the resist pattern was defined as an optimum exposure amount, and the optimum exposure amount was defined as sensitivity (mJ/cm²). The smaller the value is, the better the sensitivity is. The sensitivity was determined as “A” (extremely good) when the optimum exposure amount was less than 32 mJ/cm², “B” (good) when the optimum exposure amount was 32 mJ/cm² or more and 34 mJ/cm² or less, and “C” (poor) when the optimum exposure amount was more than 34 mJ/cm². [CDU]

By irradiation with the optimum exposure amount determined in the evaluation of sensitivity, a 25 nm contact hole pattern was formed. The formed resist pattern was observed from above the pattern using the scanning electron microscope. The variation in hole diameter was measured at 600 points in total. A 3-sigma value was determined from the distribution of the measurement values, and the 3-sigma value was defined as CDU (nm). A smaller value of CDU indicates smaller variation in hole diameter in a long period and better performance. The CDU was evaluated as “A” (extremely good) in the case of less than 2.4 nm, “B” (good) in the case of 2.4 nm or more and 2.6 nm or less, and “C” (poor) in the case of more than 2.6 nm.

### [Number of Development Defects]

The resist film was exposed at the optimum exposure amount to form a 25 nm contact hole pattern, thereby obtaining a wafer for defect inspection. The number of defects on this wafer for defect inspection was measured with use of a defect inspection device (“KLA 2810” of KLA-Tencor Corporation). The 5 number of defects after development was determined as “A” (extremely good) when the number of defects determined to be derived from the resist film was less than 30, “B” (good) when the number was 30 or more and 50 or less, and “C” (poor) when the number was more than 50.

TABLE 4

| | | | | Number of |
|---|---|---|---|---|
| | Radiation-sensitive | | | development |
| | composition | Sensitivity | CDU | defects |
| Example 1 | R-1 | B | A | B |
| Example 2 | R-2 | B | A | A |
| Example 3 | R-3 | A | A | A |
| Example 4 | R-4 | B | B | A |
| Example 5 | R-5 | A | B | A |
| Example 6 | R-6 | B | B | B |
| Example 7 | R-7 | A | B | B |
| Example 8 | R-8 | B | B | A |
| Example 9 | R-9 | B | B | A |
| Example 10 | R-10 | A | B | B |
| Example 11 | R-11 | B | A | A |
| Example 12 | R-12 | B | B | A |
| Example 13 | R-13 | B | A | B |
| Example 14 | R-14 | B | A | A |
| Example 15 | R-15 | B | A | B |
| Example 16 | R-16 | B | A | B |
| Example 17 | R-17 | A | A | A |
| Example 18 | R-18 | B | A | A |
| Example 19 | R-19 | B | A | A |
| Example 20 | R-20 | B | B | A |
| Example 21 | R-21 | B | B | A |
| Example 22 | R-22 | B | A | A |
| Example 23 | R-23 | A | B | B |
| Example 24 | R-24 | A | A | A |
| Example 25 | R-25 | A | A | A |
| Example 26 | R-26 | A | A | B |
| Example 27 | R-27 | A | A | A |
| Example 28 | R-28 | A | A | A |
| Example 29 | R-29 | B | A | A |
| Example 30 | R-30 | B | A | A |
| Example 31 | R-31 | A | B | A |
| Example 32 | R-32 | B | A | B |
| Example 33 | R-33 | B | A | A |
| Example 34 | R-34 | A | B | B |
| Example 35 | R-35 | B | A | B |
| Example 36 | R-36 | A | B | A |
| Example 37 | R-37 | A | A | A |
| Example 38 | R-38 | B | A | B |
| Example 39 | R-39 | B | B | A |
| Example 40 | R-40 | B | B | A |
| Example 41 | R-41 | B | A | A |
| Example 42 | R-42 | A | B | A |
| Example 43 | R-43 | A | B | B |
| Example 44 | R-44 | A | A | A |
| Example 45 | R-45 | B | A | B |
| Example 46 | R-46 | B | B | B |
| Comparative | CR-1 | C | C | B |
| Example 1 | | | | |
| Comparative | CR-2 | B | B | C |
| Example 2 | | | | |
| Comparative | CR-3 | C | C | C |
| Example 3 | | | | |

According to the radiation-sensitive composition and the resist pattern forming method of the present disclosure, the sensitivity, the CDU, and the number of development defects can be improved as compared with the conventional technology. Therefore, the radiation-sensitive composition and the resist pattern formation method can be suitably used for the formation of a fine resist pattern in a lithography process for various electronic devices such as semiconductor devices and liquid crystal devices.

Obviously, numerous modifications and variations of the present invention(s) are possible in light of the above teachings. It is therefore to be understood that within the scope of the appended claims, the invention(s) may be practiced otherwise than as specifically described herein.

What is claimed is:

1. A radiation-sensitive composition comprising: a polymer (A) comprising a structural unit (I) represented by formula (1); and a solvent (B),
wherein the polymer (A) comprises a radiation-sensitive acid-generating structural unit (IV), or the radiation-sensitive composition comprises a radiation-sensitive acid generator (C) other than the polymer (A), or both, and
at least one selected from the group consisting of the polymer (A) and the radiation-sensitive acid generator (C) comprises an iodo group: 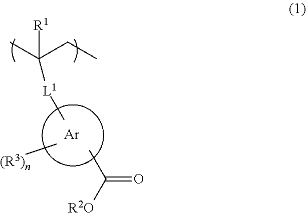
wherein in the formula (1),
R¹ is a hydrogen atom, a fluorine atom, a methyl group, or a trifluoromethyl group;
L¹ is a single bond or a divalent linking group;
Ar represents a furan ring, a thiophene ring, or an aromatic heterocycle having 9 to 20 ring members comprising a furan ring or a thiophene ring;
R² is a hydrogen atom or a monovalent organic group having 1 to 20 carbon atoms;
R³ is a halogen atom, a hydroxy group, a cyano group, a nitro group, an amino group, a thiol group, or a monovalent organic group having 1 to 20 carbon atoms; and
n is an integer of 0 to 4; and when n is 2 or more, a plurality of R³s are identical to or different from each other.

2. The radiation-sensitive composition according to claim 1, wherein the monovalent organic group having 1 to 20 carbon atoms represented by R² in the formula (1) is a group represented by formula (2): 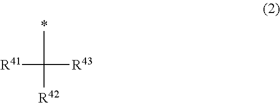
wherein in the formula (2),
R⁴¹ is a hydrogen atom or a monovalent organic group having 1 to 10 carbon atoms;
R⁴² and R⁴³ each independently represent a hydrogen atom or a monovalent organic group having 1 to 10 carbon atoms, or
R⁴² and R⁴³ taken together represent a divalent alicyclic group or heterocyclic group having 3 to 10 ring members together with the carbon atom to which R⁴² and R⁴³ are bonded; and
* represents a bond bonded to an oxygen atom in the formula (1).

3. The radiation-sensitive composition according to claim 1, wherein Ar in the formula (1) is a furan ring or a thiophene ring.

4. The radiation-sensitive composition according to claim 1, wherein the structural unit (I) comprises an iodo group.

5. The radiation-sensitive composition according to claim 1, wherein the polymer (A) further comprises a structural unit (II) having a phenolic hydroxyl group.

6. The radiation-sensitive composition according to claim 1, wherein the polymer (A) further comprises a structural unit (III) having an acid-dissociable group, the structural unit (III) being other than the structural unit (I).

7. The radiation-sensitive composition according to claim 1, wherein a content of the structural unit (I) in the polymer (A) to all structural units of the polymer (A) is 60 mol % or less.

8. The radiation-sensitive composition according to claim 1, further comprising one or more selected from the group consisting of a radiation-sensitive acid generator (C) and an acid diffusion controlling agent (D).

9. A pattern forming method, comprising
directly or indirectly applying the radiation-sensitive composition according to claim 1 to a substrate to form a resist film,
exposing the resist film to light, and
developing the exposed resist film with a developer.

10. The pattern forming method according to claim 9, wherein exposing comprises exposing the resist film to an extreme-ultraviolet ray or an electron beam.

11. A polymer comprising a structural unit (I) represented by formula (1): 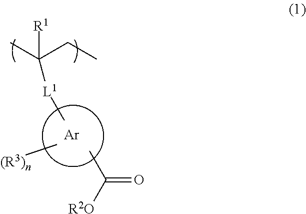
wherein in the formula (1),
R¹ is a hydrogen atom, a fluorine atom, a methyl group, or a trifluoromethyl group;
L¹ is a single bond or a divalent linking group;
Ar represents a furan ring, a thiophene ring, or an aromatic heterocycle having 9 to 20 ring members comprising a furan ring or a thiophene ring;
R² is a hydrogen atom or a monovalent organic group having 1 to 20 carbon atoms;
R³ is a halogen atom, a hydroxy group, a cyano group, a nitro group, an amino group, a thiol group, or a monovalent organic group having 1 to 20 carbon atoms;
n is an integer of 0 to 4; and when n is 2 or more, a plurality of R³s are identical to or different from each other.

12. A compound represented by formula (1-1): 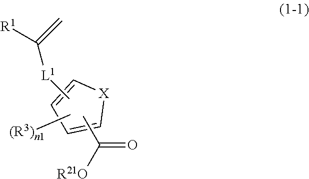
wherein in the formula (1-1),
X represents —S— or —O—;
R¹ is a hydrogen atom, a fluorine atom, a methyl group, or a trifluoromethyl group;
L¹ is a single bond or a divalent linking group;
R²¹ is an acid-dissociable group represented by a following formula (2-1), provided that when X is —S—, a total number of carbon atoms of L¹ and R²¹ is 5 or more: 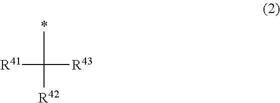
wherein in the formula (2-1),
R⁴⁴ is a hydrogen atom or a monovalent organic group having 1 to 10 carbon atoms;
R⁴⁵ and R⁴⁶ each independently represent a monovalent organic group having 1 to 10 carbon atoms, or R⁴⁵ and R⁴⁶ taken together represent a divalent alicyclic group or heterocyclic group having 3 to 10 ring members together with the carbon atom to which R⁴⁵ and R⁴⁶ are bonded,
provided that when R⁴⁴, R⁴⁵, or R⁴⁶ has a double bond other than an aromatic ring, two carbon atoms composing the double bond are bonded to 0, 2, or 3 hydrogen atoms in total; and
* represents a bond bonded to an oxygen atom,
R³ is a halogen atom, a hydroxy group, a cyano group, a nitro group, an amino group, a thiol group, or a monovalent organic group having 1 to 20 carbon atoms;
n1 is an integer of 0 to 2; and when n1 is 2, two R³s are identical to or different from each other.

13. A compound represented by formula (1-2-1) or formula (1-2-2): 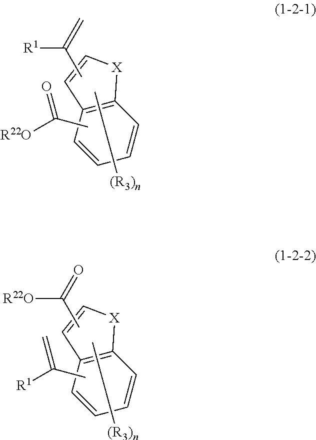
wherein in the formula (1-2-1) and the formula (1-2-2),
X represents —S— or —O—;
R¹ is a hydrogen atom, a fluorine atom, a methyl group, or a trifluoromethyl group;
R²² is an acid-dissociable group represented by a following formula (2-2), provided that a number of carbon atoms of R²² is 6 or more: 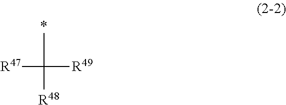
wherein in the formula (2-2),
R⁴⁷ is a hydrogen atom or a monovalent organic group having 1 to 10 carbon atoms;
R⁴⁸ and R⁴⁹ each independently represent a monovalent organic group having 1 to 10 carbon atoms, or R⁴⁸ and R⁴⁹ taken together represent a divalent alicyclic group or heterocyclic group having 3 to 10 ring members other together with the carbon atom to which R⁴⁸ and R⁴⁹ are bonded,
provided that when R⁴⁷, R⁴⁸, or R⁴⁹ has a double bond other than a double bond of an aromatic ring, two carbon atoms bonded by the double bond are bonded to 0, 1, or 3 hydrogen atoms in total; and
* represents a bond bonded to an oxygen atom,
R³ is a halogen atom, a hydroxy group, a cyano group, a nitro group, an amino group, a thiol group, or a monovalent organic group having 1 to 20 carbon atoms;
n is an integer of 0 to 4; and when n is 2 or more, a plurality of R³s are identical to or different from each other.

14. A compound represented by formula (1-3): 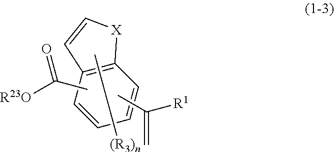
wherein in the formula (1-3),
X represents —S— or —O—;
R¹ is a hydrogen atom, a fluorine atom, a methyl group, or a trifluoromethyl group;
R²³ is a monovalent organic group having 3 or more carbon atoms;
R³ is a halogen atom, a hydroxy group, a cyano group, a nitro group, an amino group, a thiol group, or a monovalent organic group having 1 to 20 carbon atoms;
n is an integer of 0 to 4; and when n is 2 or more, a plurality of R³s are identical to or different from each other.

15. A compound represented by formula (1-4): 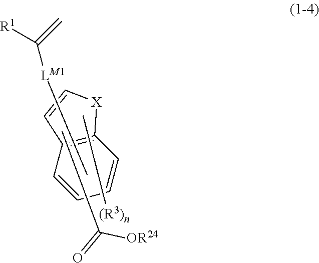
wherein in the formula (1-4),
X represents —S— or —O—;
R¹ is a hydrogen atom, a fluorine atom, a methyl group, or a trifluoromethyl group;
L^{M1} is a divalent linking group;
R²⁴ is a group represented by a following formula (2-3): 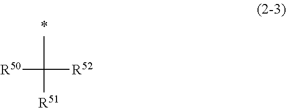
wherein in the formula (2-3),
R⁵⁰ is a hydrogen atom or a monovalent organic group having 1 to 10 carbon atoms;
R⁵¹ and R⁵² each independently represent a monovalent organic group having 1 to 10 carbon atoms, or R⁵¹ and R⁵² taken together represent a divalent alicyclic group or heterocyclic group having 3 to 10 ring members together with the carbon atom to which R⁵¹ and R⁵² are bonded; and
* represents a bond bonded to an oxygen atom,
R³ is a halogen atom, a hydroxy group, a cyano group, a nitro group, an amino group, a thiol group, or a monovalent organic group having 1 to 20 carbon atoms;
n is an integer of 0 to 4; and when n is 2 or more, a plurality of R³s are identical to or different from each other.

16. A compound represented by formula (1-5): 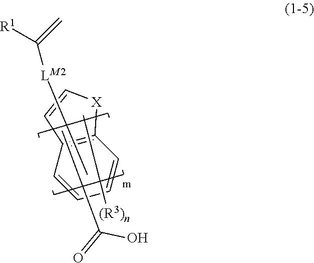
wherein in the formula (1-5),
X represents —S— or —O—;
R¹ is a hydrogen atom, a fluorine atom, a methyl group, or a trifluoromethyl group;
L^{M2} is a divalent linking group, provided that when m=0 and X is —O—, L^{M2} is a group represented by formula (3): 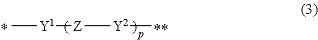
wherein in the formula (3),
Y¹ and Y² are each independently a single bond, —O—, —CO—, —CO—O—, —O—CO—, —O—CO—O—, —CO—NH—, —NH—CO—, or —S—;
Z is a divalent hydrocarbon group having 1 to 10 carbon atoms, provided that when p is 0, Y¹ is —O—, —CO—, —CO—O—, —O—CO—, —O—CO—O—, —CO—NH—, —NH—CO—, or —S—;
p is an integer of 0 to 2; and
* represents a bond bonded to a carbon atom of the vinyl group in the formula (1-5), and ** represents a bond bonded to a carbon atom of the heterocyclic ring in the formula (1-5),
R³ is a halogen atom, a hydroxy group, a cyano group, a nitro group, an amino group, a thiol group, or a monovalent organic group having 1 to 20 carbon atoms;
m is 0 or 1;
n is an integer of 0 to 4; and when n is 2 or more, a plurality of R³s are identical to or different from each other.
